(19) United States
(12) Patent Application Publication
(10) Pub. No.: US 2025/0188497 A1
(43) Pub. Date: Jun. 12, 2025

(54) STRAINS AND METHODS FOR THE CONTINUOUS PRODUCTION OF PRODUCTS BY GAS FERMENTATION

(71) Applicant: LanzaTech, Inc., Skokie, IL (US)

(72) Inventors: Sean Simpson, Auckland (NZ); Timothy Politano, Chicago, IL (US); James Clomburg, Chicago, IL (US); Jonathan Henry Massey, Des Plaines, IL (US)

(21) Appl. No.: 18/977,534

(22) Filed: Dec. 11, 2024

Related U.S. Application Data
(60) Provisional application No. 63/608,801, filed on Dec. 11, 2023.

(51) Int. Cl. C12P 5/02; C12N 1/20; C25B 15/08

(52) U.S. Cl. CPC C12P 5/026; C12N 1/20; C25B 15/081

(57) ABSTRACT

Methods and a recombinant C1-fixing microorganism for the continuous production of products from gaseous substrates. Further, the gaseous substrate comprises CO₂ and an energy source. The recombinant C1-fixing microorganism has a disruptive mutation in a membrane bound hydrogenase gene. The C1-fixing microorganism having the disruptive mutation minimizes the H₂:CO₂ uptake ratio.

### CROSS REFERENCE TO RELATED APPLICATIONS

This application claims the benefit of U.S. Provisional Patent Application No. 63/608,801 filed on Dec. 11, 2023, the entirety of which is incorporated herein by reference.

### REFERENCE TO A SEQUENCE LISTING

The application contains a Sequence Listing which has been submitted electronically in ST. 26 Sequence listing XML format and is hereby incorporated by reference in its entirety. Said ST. 26 Sequence listing XML, created on Nov. 12, 2024, is named LT297US1-Sequences.xml and is 7,384,309 bytes in size.

### BACKGROUND

The following discussion is merely provided to aid the reader in understanding the disclosure and is not admitted to describe or constitute prior art thereto.

Climate change has created an urgent demand for the development of sustainable food from renewable resources. Further, there is a need to reduce the amount of carbon dioxide and other greenhouse gas (GHG) emissions in the atmosphere, as well as to reduce water consumption and energy consumption based upon the utilization of coal, oil, and natural gas in food production systems. Recently, microbial gas fermentation has emerged as an alternative platform for the biological fixation of gases to produce single-cell protein (SCP). In particular, C1-fixing strains have been demonstrated to convert gases containing CO₂, CO, and/or H₂ into products, including nutritive compositions as food and feed ingredients. Additionally, with a constantly adjusting market, the value of the products produced by the gas fermentation process varies. When the value of the products produced by the gas fermentation are high in comparison with the cost of producing such products, it is advantageous to increase the production rate of the fermentation process. In contrast, most renewable energy sources are intermittent, not transportable, and largely dependent on the meteorological and geographical conditions. This is particularly important for places which have a high energy demand, but are restricted to a seasonally fluctuating supply of renewable energies, such as solar or wind energy. By increasing the production rate of the fermentation process at times when the market value of such products is high relative to the cost of producing such products, the economics of the fermentation process may be optimized. There is accordingly an ongoing and unmet need to develop an efficient production of nutritive food and feed ingredients by microbial fermentation of a gaseous substrate that can be produced easily from renewable resources, and which would offer a broad array of useful applications.

### SUMMARY

It is against the above background that the present disclosure provides certain advantages and advancements over the prior art.

Although this disclosure disclosed herein is not limited to specific advantages or functionalities, the disclosure provides a recombinant C1-fixing microorganism and methods for the continuous production of products and controlling the H₂:CO₂ uptake ratio, particularly by microbial fermentation of a gaseous substrate.

One embodiment is directed to recombinant C1-fixing microorganism comprising a disruptive mutation at a membrane bound hydrogenase gene locus.

The microorganism of an embodiment, wherein the disruptive mutation is a deletion.

The microorganism of an embodiment, wherein the membrane bound hydrogenase is hoxKGZ.

The microorganism of an embodiment, wherein the microorganism is selected from the group consisting of Cupriavidus necator and Ralstonia eutropha.

The microorganism of an embodiment, wherein the strain does not natively produce polyhydroxyalkanoates (PHAs).

The microorganism of an embodiment, wherein the Cupriavidus necator is selected from the group consisting of DSM 531, DSM 541, and DSM 34774.

The microorganism of an embodiment, wherein the microorganism is Cupriavidus necator DSM 34774 or a derivative thereof.

The microorganism of an embodiment, wherein the microorganism has at least 95% (e.g., 95%, 96%, 97%, 98%, 99%, 100%, or any value therebetween) sequence identity to SEQ ID NOs: 1-3. Additionally or alternatively, the microorganism of an embodiment, wherein the microorganism has at least 95% (e.g., 95%, 96%, 97%, 98%, 99%, 100%, or any value therebetween) sequence similarity to SEQ ID NOs: 1-3.

One method of an embodiment is directed to a method for the continuous production of a product, the method comprising culturing the microorganism having a disruptive mutation at a membrane bound hydrogenase in the presence of a gaseous substrate.

The method of an embodiment, wherein the gaseous substrate comprises an industrial waste product or off-gas.

The method of an embodiment, further comprising an energy source.

The method of an embodiment, wherein the energy source is provided intermittently.

The method of an embodiment, wherein the gaseous substrate comprises CO₂ and an energy source.

The method of an embodiment, wherein the energy source is H₂.

The method of an embodiment, wherein the gaseous substrate further comprises H₂, 02, or both.

The method of an embodiment, further comprising a step of limiting dissolved oxygen concentration, thereby switching a cellular burden.

The method of an embodiment, further comprising controlling iron concentrations.

An embodiment is directed to a method for obtaining a product from a circular process, wherein the method comprises: generating electric energy using a renewable and/or non-renewable source, electrolyzing water or CO₂ to produce at least H₂, O₂ or CO; passing at least one of the H₂, O₂ or CO to a bioreactor containing a culture comprising a liquid nutrient medium and a recombinant C1-fixing microorganism according to embodiment 1; and fermenting the culture to produce at least the product.

The method of an embodiment, wherein disruptive mutation is a deletion and the membrane bound hydrogenase is hoxKGZ.

The method of an embodiment, wherein the C1-fixing microorganism is selected from the group consisting of DSM 531, DSM 541, and DSM 34774.

The microorganism of an embodiment, wherein the microorganism has at least 95% (e.g., 95%, 96%, 97%, 98%, 99%, 100%, or any value therebetween) sequence identity to SEQ ID NOs: 1-3. Additionally or alternatively, the microorganism of an embodiment, wherein the microorganism has at least 95% (e.g., 95%, 96%, 97%, 98%, 99%, 100%, or any value therebetween) sequence similarity to SEQ ID NOs: 1-3.

One method of an embodiment is directed to a method for the continuous production of a product, the method comprising culturing the microorganism having a disruptive mutation at a membrane bound hydrogenase in the presence of a gaseous substrate.

The method of an embodiment, wherein the gaseous substrate comprises an industrial waste product or off-gas.

The method of an embodiment, further comprising an energy source.

The method of an embodiment, wherein the energy source is provided intermittently.

The method of an embodiment, wherein the gaseous substrate comprises CO₂ and an energy source.

The method of an embodiment, wherein the energy source is H₂.

The method of an embodiment, wherein the gaseous substrate further comprises H₂, 02, or both.

The method of an embodiment, further comprising a step of limiting dissolved oxygen concentration, thereby switching a cellular burden.

The method of an embodiment, further comprising controlling iron concentrations.

An embodiment is directed to a method for obtaining a product from a circular process, wherein the method comprises: generating electric energy using a renewable and/or non-renewable source, electrolyzing water or CO₂ to produce at least H₂, O₂ or CO; passing at least one of the H₂, O₂ or CO to a bioreactor containing a culture comprising a liquid nutrient medium and a recombinant C1-fixing microorganism according to claim 1; and fermenting the culture to produce at least the product.

One embodiment is directed to a non-naturally occurring C1-fixing strain capable of continuously growing autotrophically at up to about 40° C.

The microorganism of an embodiment, wherein the strain capable of continuously growing autotrophically at up to about 40° C. further comprises a disruptive mutation at a membrane bound hydrogenase gene locus.

In some embodiments, the microorganism is incorporated into one or more articles, converted into one or more second products, end-user products, consumer products, or any combination thereof.

In other embodiments, the composition is incorporated into pet food or animal feed. In another embodiment, the microorganism is incorporated into a product selected from a yogurt, a smoothie, a bread product, a pasta product, a nutritional bar, a chip or cracker, a plant-based meat substitute, a cheese, a plant-based cheese, a powdered nutritional supplement, a dairy product, a dairy replacement product, a meat product, a bakery product, a confection, a protein bar, a protein powder, a sport and/or energy drink, a protein shake and/or smoothie, noodles, instant noodles, a soup, an instant soup, a microwaveable food, a canned food, a freeze-dried food, a soft drink, a fruit juice drink, a vegetable drink, an infant formula, a toddler formula, a non-dairy milk, a coffee drink, a tea drink, a nutritional beverage, a powdered beverage, a nutritional supplement, a concentrated beverage, an alcoholic beverage, a cake mix, a rice cake, a flour product, chewing gum, gummies, chocolate, caramel, a cookie, chips, pretzels, crackers, biscuits, cakes, pies, a sauce, a processed seasoning, a flavor seasoning, a cooking mix, a curry, a stew, a dressing, an oils/fat, a butter, a margarine, a mayonnaise and other condiments, a lactic acid bacteria drink, an ice cream, a cream processed fish product, a processed livestock product, an agricultural canned product, a jam or marmalade, a pickled product, and a cereal or cereal product.

In one embodiment, wherein the microorganism comprises a reduced H₂:CO₂ uptake ratio by minimizing H₂ oxidation to water. In another embodiment, wherein the microorganism comprises an increase in the intracellular oxygen availability.

Another embodiment is directed to a feed or feed additive comprising the nutritive composition comprising a non-naturally occurring C1-fixing strain capable of continuously culturing autotrophically at up to about 40° C.

Culturing may also comprise growing the strain at a temperature above 30° C. and up to 40° C. For example, the temperature of the culture may be 31° C., 32° C., 33° C., 34° C., 35° C., 36° C., 37° C., 38° C., 39° C., or 40° C.

In some embodiments, wherein the nutritive composition is incorporated at a level of at least 10% to at least 30% by weight of the feed or feed additive.

In an embodiment, the composition further comprises a product selected from a yogurt, a smoothie, a bread product, a pasta product, a nutritional bar, a chip or cracker, a plant-based meat substitute, a cheese, a plant-based cheese, a powdered nutritional supplement, a dairy product, a dairy replacement product, a meat product, a bakery product, a confection, a protein bar, a protein powder, a sport and/or energy drink, a protein shake and/or smoothie, noodles, instant noodles, a soup, an instant soup, a microwaveable food, a canned food, a freeze-dried food, a soft drink, a fruit juice drink, a vegetable drink, an infant formula, a toddler formula, a non-dairy milk, a coffee drink, a tea drink, a nutritional beverage, a powdered beverage, a nutritional supplement, a concentrated beverage, an alcoholic beverage, a cake mix, a rice cake, a flour product, chewing gum, gummies, chocolate, caramel, a cookie, chips, pretzels, crackers, biscuits, cakes, pies, a sauce, a processed seasoning, a flavor seasoning, a cooking mix, a curry, a stew, a dressing, an oils/fat, a butter, a margarine, a mayonnaise and other condiments, a lactic acid bacteria drink, an ice cream, a cream processed fish product, a processed livestock product, an agricultural canned product, a jam or marmalade, a pickled product, and a cereal or cereal product.

In some embodiments, the composition is incorporated into one or more articles, converted into one or more second products, end-user products, consumer products, or any combination thereof.

In other embodiments, the composition is incorporated into pet food or animal feed.

One embodiment is directed to a method for producing a feed or feed additive comprising the nutritive composition, the method comprising: growing the non-naturally occurring strain; harvesting the strain; and incorporating the harvested strain into the feed or feed additive.

In some embodiments, the strain is grown by gas fermentation.

One embodiment is directed to a method for decreasing waste-products from commercial operations, the method comprising: growing a non-naturally occurring strain capable of continuously growing autotrophically at up to about 40° C. in the waste-products; harvesting the strain; and incorporating the harvested strain into a feed or feed additive.

In some embodiments of the method disclosed herein, the strain is a non-naturally occurring C1-fixing strain capable of continuously growing autotrophically at up to about 40° C.

In some embodiments of the strain disclosed herein, the strain is selected from the group consisting of Cupriavidus necator and Ralstonia eutropha.

The strain of an embodiment, wherein the strain does not natively produce polyhydroxyalkanoates (PHAs). The strain of an embodiment, wherein the strain does not natively produce polyhydroxybutyrate (PHBs).

The strain of an embodiment, wherein the strain continuously produces single-cell protein (SCP) from a gaseous substrate. The strain of an embodiment, wherein the strain continuously produces a product selected from the group comprising SCP and cultured protein. The strain of an embodiment, wherein the strain is continuously cultured to produce cultured protein from a gaseous substrate.

The strain of an embodiment, wherein gaseous substrate comprises CO₂ and an energy source.

The strain of an embodiment, wherein the gaseous substrate comprises CO₂, and H₂, 02, or both.

The strain of an embodiment, further comprises the SCP converted to a nutritive ingredient.

The strain of an embodiment, further comprises genetic engineering to produce enhanced nutritive products.

The strain of an embodiment, further comprises the SCP converted to a food ingredient.

The strain of an embodiment, wherein the food or food ingredient is selected from a yogurt, a smoothie, a bread product, a pasta product, a nutritional bar, a chip or cracker, a plant-based meat substitute, a cheese, a plant-based cheese, a powdered nutritional supplement, a dairy product, a dairy replacement product, a meat product, a bakery product, a confection, a protein bar, a protein powder, a sport and/or energy drink, a protein shake and/or smoothie, noodles, instant noodles, a soup, an instant soup, a microwaveable food, a canned food, a freeze-dried food, a soft drink, a fruit juice drink, a vegetable drink, an infant formula, a toddler formula, a non-dairy milk, a coffee drink, a tea drink, a nutritional beverage, a powdered beverage, a nutritional supplement, a concentrated beverage, an alcoholic beverage, a cake mix, a rice cake, a flour product, chewing gum, gummies, chocolate, caramel, a cookie, chips, pretzels, crackers, biscuits, cakes, pies, a sauce, a processed seasoning, a flavor seasoning, a cooking mix, a curry, a stew, a dressing, an oils/fat, a butter, a margarine, a mayonnaise and other condiments, a lactic acid bacteria drink, an ice cream, a cream processed fish product, a processed livestock product, an agricultural canned product, a jam or marmalade, a pickled product, and a cereal or cereal product.

The microorganism of an embodiment, wherein the microorganism is Cupriavidus necator DSM 34774.

The strain of an embodiment, wherein the strain comprises SEQ ID NOs: 1-3.

For the purposes of the present disclosure, the C. necator strain utilized for producing a nutritive ingredient may have a genome comprising or consisting of the nucleic acid sequence of SEQ ID NOs: 1-3 or a genome comprising or consisting of a nucleic acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NOs: 1-3. The non-naturally occurring C1-fixing strain utilized for producing SCP may also be the strain deposited under accession number DSM 34774, or a strain sharing substantial phenotypic characteristics (e.g., temperature resistance) or genotypic characteristics (e.g., a genome with at least 80% sequence identity to SEQ ID NOs: 1-3) with a strain deposited under accession number DSM 34774. Additionally or alternatively, the C. necator strain utilized for producing a nutritive ingredient may have a genome comprising or consisting of a nucleic acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% similarity to SEQ ID NOs: 1-3.

The C. necator strain having identification reference number DSM 34774 was deposited under the provisions of the Budapest Treaty at the German Collection of Microorganisms (DSM) located at Inhoffenstraße 7B, 38124 Braunschweig, Science Campus Braunschweig-Süd, Germany on Oct. 6, 2023. All restrictions on the availability to the public of the deposited material will be irrevocably removed upon the granting of a patent from the above-identified application. The deposited cultures will be replaced should they die or be destroyed during the enforceable life of any patent issued out of this patent application, for five years after the last request for a sample of the deposited microorganism or for a term of at least thirty (30) years. Samples will be stored under agreements that would make them available beyond the enforceable life of the patent for which the deposit was made.

Without being bound by theory, specific examples of genetic modifications that may provide temperature resistance in the non-naturally occurring C1-fixing strain include, but are not limited to, mutations in a hydrogenase regulator (L405H (cTt→cAt) mutation in HoxA encoded by hoxA, E6A55_32285), deletions or frame shifts within certain regions (Δ35433 bp encompassing genes [E6A55_33395]-[E6A55_33560], the open reading frames of which are shown in FIG. 4), or mutations in other genes associated with autotrophic growth.

Phenotypic features that the non-naturally occurring C1-fixing strain may possess include, but are not limited to, a growth rate that is at least 20%, at least 25%, at least 30%, at least 35%, at least 40%, at least 45%, at least 50%, at least 55%, as at least 60%, at least 65%, at least 70%, at least 75%, at least 80%, at least 85%, at least 90%, at least 95%, or at least 100% higher than the growth rate of a wild type or naturally occurring strain of C. necator at temperatures above 30° C. For example the growth rate may be at least 20%, at least 25%, at least 30%, at least 35%, at least 40%, at least 45%, at least 50%, at least 55%, as at least 60%, at least 65%, at least 70%, at least 75%, at least 80%, at least 85%, at least 90%, at least 95%, or at least 100% higher than the growth rate of a wild type or naturally occurring strain of C. necator at 31° C., 32° C., 33° C., 34° C., 35° C., 36° C., 37° C., 38° C., 39° C., or 40° C. or within a range of 32-40° C., 33-40° C., 34-40° C., 35-40° C., 31-39° C., 32-39° C., 33-39° C., 34-39° C., or 35-39° C. Optionally, the improved growth at high temperatures may be observed when the C. necator is cultured in autotrophic conditions.

The strain of an embodiment, wherein the strain does not comprise an exogenous nucleic acid.

The strain of an embodiment, the SCP of the nutritive ingredient has a higher concentration of amino acids, oligopeptides, polypeptides, or derivatives thereof, as compared to a SCP of nutritive ingredient not having this strain.

The strain of some embodiments, the nutritive ingredient has higher crude protein content as compared to a nutritive ingredient not having the strain.

In another embodiment, crude protein in the strain is at least 20% to 80% of the strain mass. In some embodiments, crude protein in the strain is at least 40% to 70% of the strain mass.

The strain of an embodiment, further comprises genetic engineering to produce products.

The strain of an embodiment, wherein the strain comprises an exogenous nucleic acid.

One embodiment is directed to a method for the continuous production of single-cell protein (SCP), the process comprising: passing a gaseous substrate to a bioreactor containing a culture of a non-naturally occurring C1-fixing strain, in a culture medium such that the strain converts the gaseous substrate to SCP under autotrophic conditions; and recovering the SCP from the bioreactor.

The method of an embodiment, wherein the gaseous substrate comprises an industrial waste product or off-gas.

The method of an embodiment, further comprising an energy source.

The method of an embodiment, wherein the energy source is provided intermittently.

The method of an embodiment, wherein the gaseous substrate comprises CO₂ and an energy source.

The method of an embodiment, wherein the energy source is H₂.

The method of an embodiment, wherein the gaseous substrate further comprises H₂, 02, or both.

The method of an embodiment, further comprising a step of limiting dissolved oxygen concentration, thereby switching a cellular burden.

The method of an embodiment, further comprising controlling iron concentrations.

One embodiment is directed to a method for the continuous production of a food or feed ingredient, the process comprising: passing a gaseous substrate to a bioreactor containing a culture of a non-naturally occurring C1-fixing strain, in a culture medium such that the strain converts the gaseous substrate to SCP under autotrophic conditions; recovering the SCP from the bioreactor; and utilizing the SCP as an ingredient in food or feed.

Another embodiment is directed to a method for obtaining a nutritive product from a circular process, wherein the method comprises: a) generating electric energy using a renewable and/or non-renewable source, b) electrolyzing water or CO2 to produce at least H₂, O₂ or CO; c) passing at least one of the H₂, O₂ or CO to a bioreactor containing a culture comprising a liquid nutrient medium and a non-naturally occurring C1-fixing strain; and d) fermenting the culture to produce at least the nutritive product.

The method of an embodiment, wherein the electrolyzing has a cost per unit electric energy.

The method of an embodiment, further comprising passing at least a portion of the O₂ produced in the electrolysis process to a combustion or gasification process to produce the carbon dioxide.

The method of an embodiment, wherein electrolyzing is operated to supplement a C1 feedstock during time periods when the cost per unit electric energy is less than the cost per unit of C1 feedstock.

The method of an embodiment, wherein the renewable energy source comprises solar energy, wind power, wave power, tidal power, hydro power, geothermal energy, biomass and/or biofuel combustion, nuclear, or any combination thereof.

The method of an embodiment, wherein one or more of steps a), b), or c) are intermittent.

One embodiment is directed to a system for producing a nutritive product comprising: a) an electrolysis process in fluid communication with a renewable and/or non-renewable energy source for producing at least one of H2, O2, or CO; b) an industrial plant for producing at least C1 feedstock; and c) a bioreactor, in fluid communication with the electrolysis process and/or in continuous fluid communication with the industrial plant, comprising a reaction vessel suitable for growing, fermenting, and/or culturing and housing the non-naturally occurring C1-fixing strain to produce at least the nutritive product.

The system of an embodiment, further comprising at least one oxygen enriched combustion or gasification unit in fluid communication with the electrolysis process, the bioreactor, or both, the oxygen enriched combustion or gasification unit for producing carbon dioxide.

The system of an embodiment, further comprising at least one downstream processing system in fluid communication with the bioreactor selected from a recovery system, a purification system, an enriching system, a storage system, a recycling or further processing system for fermentation off-gas, hydrogen, water, oxygen, carbon dioxide, used medium and medium components, or combinations thereof.

The system of an embodiment, further comprising a cell processing unit, in fluid communication with the bioreactor, wherein the non-naturally occurring C1-fixing strain is further processed to a single cell protein (SCP) and/or a cell-free protein synthesis platform.

The system of an embodiment, wherein the renewable energy source is selected from solar energy, wind power, wave power, tidal power, hydro power, geothermal energy, biomass and/or biofuel combustion, nuclear, or any combination thereof.

One embodiment is directed to a two-stage fermentation method for producing a nutritive product, wherein the method comprises: a) culturing a first microorganism under conditions wherein the first microorganism ferments a first feedstock to produce an intermediate; and b) culturing a second microorganism under conditions wherein the second microorganism ferments the intermediate to produce the nutritive product from the intermediate; and wherein at least one of the first microorganism or the second microorganism is selected from a non-naturally occurring C1-fixing strain.

The method of an embodiment, further comprising a methanogen.

The method of an embodiment, wherein a carbon dioxide metabolizing microorganism comprise a microorganism selected from mesophilic methanotrophic bacterium, mesophilic bacterium, and cyanobacteria.

One embodiment is directed to a two-step fermentation method for producing a product comprising culturing a first microorganism under conditions wherein the first microorganism ferments a first feedstock to produce an intermediate and culturing a second microorganism under conditions wherein the second microorganism ferments a second feedstock to produce the product from the intermediate.

In some aspects of the method disclosed herein, the first feedstock or the second feedstock is a gaseous substrate.

In some aspects of the method disclosed herein, the gaseous substrate comprises one or more of CO, CO₂, H₂, and CH₄.

In some aspects of the method disclosed herein, the first feedstock or the second feedstock is a carbohydrate.

In some aspects of the method disclosed herein, the carbohydrate comprises one or more of xylose, arabinose, glucose, fructose, mannose, galactose, fucose, sucrose, maltose, melibiose, xylan, xylogluco-oligosaccharides, and mannitol.

In some aspects of the method disclosed herein, the first microorganism or the second microorganism is a C1-fixing microorganism.

In some aspects of the method disclosed herein, the carbohydrate-fermenting microorganism is selected from the group consisting of Escherichia coli, Bacillus subtilis, Caldicellulosiruptor saccharolyticus, Clostridium acetobutylicum, Clostridium beijerinckii, Lactococcus lactis, Lactobacillus, Klebsiella, Thermoplasma acidophilum, Picrophilus torridus, Zymomonas mobilis, Saccharomyces cerevisiae, Schizosaccharomyces pombe, Schwanniomyces (Debaryomyces) occidentalis, Kluyveromyces marxianus, and Yarrowia lipolytica.

In some aspects of the method disclosed herein, the first microorganism and the second microorganism are cultured in one bioreactor.

In some aspects of the method disclosed herein, the first microorganism is cultured in a first bioreactor to produce the intermediate, and the second microorganism is cultured in a second bioreactor to produce the product from the intermediate.

In some aspects of the method disclosed herein, at least a portion of an intermediate produced in the first bioreactor is passed to the second bioreactor.

In some aspects of the method disclosed herein, the carbohydrate-fermenting microorganism further produces CO₂.

In some aspects of the method disclosed herein, CO₂ produced by the carbohydrate-fermenting microorganism is a substrate for a C1-fixing microorganism.

In one aspect, there is provided a process having enhanced production efficiency, the process comprising (i) providing a first feedstock to a first microorganism and culturing the microorganism under conditions to produce an intermediate and (ii) providing a second feedstock to a second microorganism and culturing the microorganism under conditions to convert the intermediate to a product. As used herein, production of an intermediate as described in (i) is referred to as “step 1,” and conversion of the intermediate to produce a product as described in (ii) is referred to as “step 2.” The product produced by step 2 may also be referred to as a “final product” herein.

In some embodiments, the first feedstock is a gaseous substrate, such as a C1 feedstock, and the first microorganism is a C1-fixing microorganism (e.g., for step 1). In some embodiments, the second feedstock is a carbohydrate, and the second microorganism is a carbohydrate-fermenting microorganism (e.g., for step 2).

In an alternative embodiment, the first feedstock is a carbohydrate, and the first microorganism is a carbohydrate-fermenting microorganism (e.g., for step 1). In another alternate embodiment, the second feedstock is a gaseous C1 feedstock, and the second microorganism is a C1-fixing microorganism (e.g., for step 2).

In some embodiments, step 1 and step 2 occur in a single bioreactor. In some embodiments, the first feedstock and second feedstock are provided to a bioreactor comprising a co-culture of a first microorganism and a second microorganism. In some embodiments, the co-culture comprises a C1-fixing microorganism and a carbohydrate-fermenting microorganism. In some embodiments, the first feedstock, a gaseous C1 feedstock, is converted to an intermediate by the C1-fixing microorganism, and the second feedstock, a carbohydrate feedstock, is consumed by the carbohydrate-fermenting microorganism to convert the intermediate to a final product.

In some embodiments, step 1 occurs in a first bioreactor, and step 2 occurs in a second bioreactor. For example, the first bioreactor may comprise a C1-fixing microorganism and a gaseous C1 feedstock, and the second bioreactor may comprise a carbohydrate-fermenting microorganism and a carbohydrate feedstock. In some embodiments, at least a portion of the intermediate produced by step 1 is recovered from the first bioreactor and transferred to the second bioreactor, where the intermediate is converted to a final product by the second microorganism. In some embodiments, the permeate from step 1 (media without cells) is transferred from the first bioreactor to the second bioreactor. In other embodiments, a portion or all of the contents of the first bioreactor (media with cells) is transferred to the second bioreactor comprising the second microorganism supplied with the second feedstock.

In certain aspects of the disclosure, the combined stream can be returned to the first bioreactor to supplement the liquid nutrient medium being continuously added. In certain embodiments, it may be desirable to further process the recycle stream to remove un-desired by-products of the fermentation of step 2. In certain embodiments, the pH of the recycle stream may be adjusted, and further vitamins and metals added to supplement the stream.

One embodiment is directed to an integrated system for converting gaseous carbon compounds to carbon neutral or negative products comprising: a source capable of producing a mix of gaseous carbon feedstock which includes carbon dioxide and methane; a biological methane processing system containing methane metabolizing microorganisms and fed with methane from the source, the biological methane processing system arranged to: propagate methane metabolizing microorganisms; and, produce carbon dioxide as a by-product; a biological carbon dioxide processing system containing carbon dioxide metabolizing microorganisms and fed with carbon dioxide from the source of mixed gaseous carbon feedstock and the carbon dioxide by-product, the biological carbon dioxide processing system arranged to: propagate carbon dioxide metabolizing microorganisms; and a microorganism harvesting and processing system arranged to harvest the propagated microorganisms and produce single-cell protein.

Another embodiment is directed to an integrated biological method for converting gaseous carbon compounds to carbon neutral or negative products comprising: feeding gaseous methane from a first biogas source to a biological methane processing system capable of propagating methanotrophic bacterium and producing carbon dioxide as a by-product; feeding (a) gaseous carbon dioxide from the first biogas source, and (b) the carbon dioxide by-product, to a biological carbon dioxide processing system capable of propagating a carbon dioxide metabolizing bacterium; and producing a single-cell protein from the propagated methanotrophic bacterium and the propagated carbon dioxide metabolizing bacterium.

An embodiment is directed to a method of circular economy comprising producing a feedstock from a source; converting the feedstock to a single-cell protein; and utilizing the single-cell protein by the source.

In an embodiment, wherein the converting comprises culturing a non-naturally occurring strain at up to about 40° C. thereby producing single-cell protein.

One embodiment is directed to a method of circular economy comprising: producing biogas from waste products excreted by a plurality of animals wherein the biogas includes a mixture of gaseous methane and carbon dioxide; converting the biogas to single-cell proteins; and feeding the plurality of animals with food comprising the single-cell proteins.

Another embodiment is directed to a method for producing a protein-based bioplastic, wherein the method comprises: a) culturing a non-naturally occurring C1-fixing strain according to claim 1 in a nutrient medium in the presence of a gaseous substrate to produce single-cell protein (SCP); and b) processing the SCP to produce a protein-based bioplastic.

The method of an embodiment, wherein the processing step comprises one or more of sterilizing the SCP, centrifuging the SCP, drying the SCP, denaturing the SCP, and extracting the SCP.

The method of an embodiment, wherein the processing step comprises blending the SCP with a plasticizer.

The method of an embodiment, wherein the plasticizer is one or more of water, glycerol, ethylene glycerol, propylene glycerol, palmitic acid, diethyl tartarate, dibutyl tartarate, 1,2-butanediol, 1,3-butanediol, polyethylene glycol (PEG), sorbitol, mantitoletc, dimethylaniline, diphenylamine, and 2,3-butanediol.

The strain of some embodiments, wherein the products are selected from ethanol, acetate, 1-butanol, butyrate, 2,3-butanediol, lactate, butene, butadiene, methyl ethyl ketone, acetone, lipids, 3-hydroxyproprionate, terpenes, isoprene, fatty acids, 2-butanol, 1,2-propanediol, 1-propanol, 1-hexanol, 1-octanol, chorismite-derived products, 3-hydroxybutyrate, 1,3-butanediol, 2-hydroxyisobutyrate, 2-hydroxyisobutyric acid, isobutylene, adipic acid, keto-adipic acid, 1,3-hexanediol, 3-methyl-2-butanol, 2-buten-1-ol, isovalerate, isoamyl alcohol, a fatty alcohol, a carotenoid (e.g., a terpene), omega-3 fatty acids, omega-6 fatty acids, alkenes (such as ethylene), and polyphenols, or any combination thereof.

The strain of an embodiment, wherein the strain produces a commodity chemical

The strain of an embodiment, wherein the first product SCP is incorporated into one or more articles, converted into one or more second products, end-user products, consumer products, or any combination thereof.

The disclosure further provides the non-naturally occurring C1-fixing strain, further comprising a nutritive ingredient and at least one excipient.

The disclosure further provides the nutritive ingredient, wherein the ingredient is suitable for feeding to one or more of beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents.

The strain of an embodiment, wherein the nutritive ingredient is suitable for incorporating into pet food.

The strain of an embodiment, wherein the nutritive ingredient is suitable for incorporating into food for humans.

The disclosure further provides the non-naturally occurring C1-fixing strain, wherein the strain is suitable as a single cell protein (SCP).

The disclosure further provides the non-naturally occurring C1-fixing strain, wherein the strain is suitable as a cell-free protein synthesis (CFPS) platform.

The disclosure further provides the non-naturally occurring C1-fixing strain, wherein the product is native to the strain.

In some aspects of the method disclosed herein, the substrate comprises one or more of CO, CO₂, and H₂.

In some embodiments, both anaerobic and aerobic gases can be used to feed separate cultures (e.g., an anaerobic culture and an aerobic culture) in two or more different bioreactors that are both integrated into the same process stream.

In some embodiments, the disclosure provides a method for improving the performance and/or the economics of a fermentation process, the fermentation process defining a bioreactor containing a bacterial culture in a liquid nutrient medium, wherein the method comprises passing a C1 feedstock comprising one or both of CO and CO₂ from an industrial process to the bioreactor, wherein the C1 feedstock has a cost per unit, intermittently passing at least one of H₂, O₂, or CO from the electrolysis process to the bioreactor, wherein the electrolysis process has a cost per unit, and fermenting the culture to produce one or more fermentation products, wherein each of the one or more fermentation products has a value per unit. In certain instances, multiple electrolysis processes are utilized in order to provide one or all of CO, CO₂, and H₂ to the bioreactor.

In another embodiment, the local power grid provides electricity intermittently passed as electrical energy produced by power based on availability of electrical power or the availability of electricity below a threshold price, where power prices fall as demand falls, or as set by the local power grid.

In an embodiment, the disclosure can be operated intermittently, where product conversion can be intermittent during periods when an electricity grid is oversupplied with electricity, or idle when electricity is scarce or power is in demand. The disclosure provides a process that is capable of being fine-tuned to assist with balancing an electrical power grid system.

In one embodiment an autotrophic strain intermittently consumes, in part or entirely, the energy provided by the availability of power.

In some embodiments, the hydrogen in the stream comprises hydrogen selected from green hydrogen, blue hydrogen, grey hydrogen, pink hydrogen, turquoise hydrogen, yellow hydrogen, white hydrogen, or any combination thereof.

Bacterial strains according to the disclosure can be evolved using adaptive laboratory evolution methods to improve heat tolerance and improve growth and biosynthesis in autotrophic conditions. However, it is understood by those skilled in the art and for the purposes of the present disclosure that ALE is not the only methodology that can be used to prepare a temperature resistant strain of C. necator, but merely one example of a suitable method.

ALE refers to the culture of cells or organisms under defined conditions leading to adaptive changes that accumulate in populations of cells or (microbial) organisms during selection under specified growth conditions. In particular, the desired trait is selected in an evolution environment where it provides a fitness benefit. In the target environment, the desired trait is exploited (for instance, heat resistance at elevated temperature conditions). In the target environment, the desired trait allows the increase or decrease of at least one desired trait and/or avoids the increase or decrease of at least one trait. In one specific embodiment, in the target environment the desired trait allows the increase in the production flux of a least one desired product or survival advantage.

Typically, a metabolic trait or a phenotypic trait of interest evolves over several generations of the cell or organism. This may include at least two generations, e.g., at least 10, at least 50, at least 100, at least 200, at least 300 or more generations, preferably at least 50 generations, more preferably about 100 generations or more of the cell or organism are necessary to evolve a metabolic trait. Thus, the cells or organisms are cultured for a certain time in a desired environment for, such as several days, weeks, months or years.

The target environment should be suitable to evolve a desired trait. When the population of cells or organisms is cultured in the target environment, a part of the population of the cell or organism will establish the desired trait, while some of the cells or organisms will die. In this way, the desired trait is selected. Preferably, at least 5%, at least 10%, at least 20%, at least 30%, at least 40%, at least 50%, at least 60%, at least 70%, at least 80%, at least 90% or more of the population have established the desired metabolic or phenotypic trait.

For the purposes of the present application, continuous ALE can be used to obtain a heat-resistant strain of C. necator capable of growth and culture at elevated temperatures, optionally in autotrophic conditions. As one example of continuous ALE, Example 1 of this disclosure describes inoculating a continuous-flow stirred tank reactor (CSTR) using a base strain of C. necator (H16 PHB-4). The culture can be continuously grown on a minimal autotrophic media with H2/O2/CO2 inputs. Once the culture is established and growth was maintained at a steady state, the reactor temperature was increased step-wise and held to allow the culture to adapt to the increased temperature. These step wise changes may be continued over the course of several days; for example, 1-3 days, 3-50 days, 50-100 days, 50 days to 1 year, or 1-5 years.

The pH can be kept between pH 4 and 12, preferably between pH 6 and 9, particularly preferably between pH 7 and 8.

For the purposes of the present disclosure, ALE can be operated batchwise, semi-batchwise or continuously.

Some embodiments provide the use of naturally occurring or engineered microorganisms to convert CO₂ gas and/or syngas and/or producer gas and/or methane to higher value mid- to long-carbon chain length amino acids, proteins, and other biological nutrients. One embodiment allows the development of new natural or classically bred and/or genetically enhanced strains of microorganisms that can be used for syngas bioprocessing within biological gas-to-chemical (GTC) processes to produce and/or secrete various relatively long chain organic compounds that are drop-in, and are currently only produced in bulk from higher plant agricultural crops or animal sources.

An embodiment relates to the selection and/or breeding and/or engineering of microorganisms, including but not limited to hydrogen-oxidizing, carbon monoxide-oxidizing, and knallgas microorganisms, with a natural capability to grow and synthesize biomass on gaseous carbon sources such as syngas and/or CO₂, such that the production microorganisms synthesize targeted chemical products under gas cultivation. The microorganisms and methods of the present disclosure can enable low cost synthesis of biochemicals, which can compete on price with petrochemicals and higher-plant derived amino acids, proteins, and other biological nutrients. In certain embodiments, these amino acids, proteins, and other biological nutrients are predicted to have a substantially lower price than amino acids, proteins, and other biological nutrients produced through heterotrophic or microbial phototrophic synthesis.

An embodiment relates to a composition comprising a microorganism that converts syngas and/or gaseous CO₂ and/or a mixture of CO₂ gas and H₂ gas along with a nitrogen source including but not limited to ammonia, ammonium, and/or urea, into one or more amino acids, proteins, and other biological nutrients. In some embodiments, the composition comprises a microorganism, wherein the microorganism is one or more of the following: a hydrogen-oxidizing chemoautotrophic microorganism; a carbon monoxide-oxidizing microorganism; a knallgas microorganism. Knallgas microbes, hydrogenotrophs, carboxydotrophs, and chemoautotrophs more broadly, are able to capture CO₂ or CO as their sole carbon source to support biological growth. In some embodiments, this growth includes the biosynthesis of amino acids and proteins. Knallgas microbes and other hydrogenotrophs can use H₂ as a source of reducing electrons for respiration and biochemical synthesis. In some embodiments knallgas organisms and/or hydrogenotrophs and/or carboxydotrophs and/or other chemoautotrophic microorganisms are grown on a stream of gasses including but not limited to one or more of the following: CO₂; CO; H₂; along with inorganic minerals dissolved in aqueous solution. In some embodiments knallgas microbes and/or hydrogenotrophs and/or carboxydotrophs and/or other chemoautotrophic and/or methanotrophic microorganisms convert greenhouse gases (GHG's) into biomolecules including amino acids and proteins.

In certain embodiments, well known drawbacks of photosynthetic systems for capture and conversion of CO₂ such as those based on algae or higher plants are circumvented, while the unique biological capability, evolved over billions of years, for complex organic synthesis from CO₂ to produce valuable biochemicals such as but not limited to amino acids and proteins, is still leveraged.

In one embodiment, a natural or engineered microorganism is provided that is capable of converting a gaseous substrate such as producer gas or synthesis gas or another gas mixture that contains H₂ and CO₂, and/or CO, and/or CH₄ into amino acids, proteins, and other biological nutrients. The gaseous substrate is used by the microorganism as a carbon and/or energy source. In some embodiments, microorganisms that are capable of growing on a gaseous substrate are transformed with a polynucleotide that encodes a gene that is required for biosynthesis of an amino acid, protein, or other biological nutrient. In some embodiments, an amino acid, protein, other biological nutrient, or a whole cell product is recovered from the microbial cells or from a microbial growth medium. Producer gas, which may be used in the microbial growth processes described herein, may come from sources that include gasification of waste feedstock and/or biomass residue feedstock, or waste gas from industrial processes or steam reforming of natural gas or biogas.

In one aspect, a non-naturally occurring microorganism is provided that is capable of growing on a gaseous substrate as a carbon and/or energy source, and wherein the microorganism includes at least one exogenous nucleic acid. In some embodiments, the microorganism is a bacterial cell. For example, in some embodiments, the bacterial cell is a Cupriavidus sp. or Ralstonia sp., for example, but not limited to, Cupriavidus necator. In some non-limiting embodiments, the microorganism is Cupriavidus necator DSM 34774. In some non-limiting embodiments, the microorganism is Cupriavidus necator DSM 531, DSM 541, or DSM 34774.

In some embodiments, the gaseous substrate includes CO₂ as a carbon source. In some embodiments, the gaseous substrate includes H₂ and/or O₂ as an energy source. In some embodiments, the gaseous substrate includes producer gas, syngas, or pyrolysis gas. In some embodiments, the gaseous substrate includes a mixture of gases, comprising H₂ and/or CO₂ and/or CO.

In some embodiments, the microorganism produces amino acids, proteins, and other biological nutrients when cultured in the presence of the gas substrate under conditions suitable for growth of the microorganism and production of bioproducts.

In some embodiments, an exogenous gene is encoded by a coding sequence in the non-naturally occurring microorganism that is carried on a broad-host-range plasmid. In some embodiments, the exogenous gene coding sequence is under the control of a non-native inducible promoter. In some embodiments, the inducible promoter is derived from the E. coli ara operon.

In some embodiments, the coding sequence (CDS) of the exogenous gene is codon optimized for expression in a microorganism of as described herein, for example, but not limited to a Ralstonia or Cupriavidus species, for example, Cupriavidus necator.

In another embodiment, methods are provided for producing amino acids, proteins, and other biological nutrients using an engineered microorganism as described herein that is capable of growing on a gaseous substrate as a carbon and/or energy source, and that includes at least one exogenous nucleic acid. In some embodiments, a non-naturally occurring microorganism as described herein is cultured in a bioreactor that includes a gaseous substrate and a culture medium (e.g., a liquid growth medium) that includes other nutrients for growth and bioproduct production, under conditions that are suitable for growth of the microorganism, wherein the microorganism produces amino acids, proteins, and other biological nutrients.

In some embodiments, the gaseous substrate in the bioreactor includes H₂ and/or CO₂. In some embodiments, the gaseous substrate is producer gas, syngas, or pyrolysis gas. In some embodiments, the gaseous substrate is natural gas or biogas. In some embodiments, the gaseous substrate is derived from municipal solid waste, black liquor, agricultural waste, wood waste, stranded natural gas, biogas, sour gas, methane hydrates, tires, pet coke, sewage, manure, straw, lignocellulosic energy crops, lignin, crop residues, bagasse, saw dust, forestry residue, food waste, waste carpet, waste plastic, landfill gas, and/or lignocellulosic biomass.

In some embodiments, amino acids, proteins, and other biological nutrients are recovered from the culture medium. In some embodiments, the culture medium is a biphasic liquid medium that includes an aqueous phase and an organic phase, and amino acids, proteins, and/or other biological nutrients are recovered by extraction or reactive extraction in the organic phase.

In another aspect, microorganisms and methods for producing amino acids, proteins, and other biological nutrients are provided. In some embodiments, a natural or non-naturally occurring microorganism is provided that is capable of growing on a gaseous substrate as a carbon and/or energy source, wherein the microorganism includes zero or at least one exogenous nucleic acid, and wherein said microorganism biosynthesizes amino acids, proteins, and other biological nutrients. In some embodiments, a method is provided for producing amino acids, proteins, and other biological nutrients in a naturally or non-naturally occurring microorganism as described herein that is capable of growing on a gaseous substrate as a carbon and/or energy source, that includes zero or one or more exogenous nucleic acids, and that biosynthesizes amino acids, proteins, and other biological nutrients, including culturing the naturally or non-naturally occurring microorganism in a bioreactor that includes a gaseous substrate and a culture medium (e.g., a liquid growth medium) that includes other nutrients for growth and bioproduct production, under conditions that are suitable for growth of the microorganism and production of amino acids, proteins, and other biological nutrients, wherein the microorganism produces amino acids, proteins, and other biological nutrients.

In some embodiments, the microorganisms of the present disclosure are used to capture CO₂ from industrial flue gasses and produce a protein-rich biomass. In some embodiments, this protein-rich biomass is a commodity. In some embodiments, the protein-rich biomass is used as a single cell protein (SCP). In some embodiments, the protein-rich biomass is used as an aquaculture feed or in an aquaculture feed formulation or in a fertilizer.

In some embodiments, the protein-rich biomass is used as a high-protein substitute for fishmeal used in aquaculture and/or other animal feed and/or plant fertilizer products. In some non-limiting embodiments, the present disclosure is used both for GHG reduction and to produce high-protein products for applications including but not limited to animal feed or replacements for fish meal, casein, whey, or soy meal.

In one aspect, a biological and chemical method is provided for the capture and conversion of an inorganic and/or organic molecules containing only one carbon atom, into organic molecules containing two or more carbon atoms produced through anabolic biosynthesis comprising: introducing inorganic and/or organic molecules containing only one carbon atom, into an environment suitable for maintaining chemoautotrophic microorganisms; introducing a gaseous substrate into an environment suitable for maintaining chemoautotrophic microorganisms; wherein the inorganic and/or organic molecules containing only one carbon atom are used as a carbon source by the microorganism for growth and/or biosynthesis; converting the inorganic and/or organic molecules containing only one carbon atom into the organic molecule products containing two or more carbon atoms within the environment via at least one chemosynthetic carbon-fixing reaction and at least one anabolic biosynthetic pathway contained within the chemoautotrophic microorganisms; wherein the chemosynthetic fixing reaction and anabolic biosynthetic pathway are at least partially driven by chemical and/or electrochemical energy provided by electron donors and electron acceptors that have been generated chemically and/or electrochemically and/or thermochemically and/or are introduced into the environment from at least one source external to the environment.

In some embodiments, said electron donors and/or molecules containing only one carbon atom are generated through a thermochemical process acting upon organic matter comprising at least one of: gasification; pyrolysis; steam reforming; autoreforming. In some embodiments, said electron donors and/or organic molecules containing only one carbon atom are generated through methane steam reforming. In some embodiments, the ratio of hydrogen to carbon monoxide in the output gas from gasification and/or pyrolysis and/or autoreforming and/or steam reforming is adjusted using the water gas shift reaction prior to the gas being delivered to the microorganisms.

In some embodiments, said electron donors and/or electron acceptors are generated or recycled using renewable, alternative, or conventional sources of power that are low in greenhouse gas emissions, and wherein said sources of power are selected from at least one of photovoltaics, solar thermal, wind power, hydroelectric, nuclear, geothermal, enhanced geothermal, ocean thermal, ocean wave power, and tidal power.

In some embodiments, said electron donors and/or electron acceptors are generated using grid electricity during periods when electrical grid supply exceeds electrical grid demand, and wherein storage tanks buffer the generation of said electron donors and/or electron acceptor, and their consumption in the chemosynthetic reaction.

In some embodiments, molecular hydrogen acts as an electron donor and is generated via a method using at least one of the following: electrolysis of water; thermochemical splitting of water; electrolysis of brine; electrolysis and/or thermochemical splitting of hydrogen sulfide. In some embodiments, electrolysis of water for the production of hydrogen is performed using one or more of the following: Proton Exchange Membranes (PEM), liquid electrolytes such as KOH, alkaline electrolysis, Solid Polymer Electrolyte electrolysis, high-pressure electrolysis, high temperature electrolysis of steam (HTES). In some embodiments, thermochemical splitting of water for the production of hydrogen is performed using one or more of the following: the iron oxide cycle, cerium(IV) oxide-cerium(III) oxide cycle, zinc zinc-oxide cycle, sulfur-iodine cycle, copper-chlorine cycle, calcium-bromine-iron cycle, hybrid sulfur cycle.

In some embodiments, molecular hydrogen acts as an electron donor and is generated via electrochemical or thermochemical processes known to produce hydrogen with low- or no-carbon dioxide emissions including one or more of the following: carbon capture and sequestration (CCS) enabled methane steam reforming; CCS enabled coal gasification; the KvoTrner-process and other processes generating a carbon-black product; CCS enabled gasification or pyrolysis of biomass; pyrolysis of biomass producing a biochar co-product.

In some embodiments, said electron donors include but are not limited to one or more of the following reducing agents: ammonia; ammonium; carbon monoxide; dithionite; elemental sulfur; hydrocarbons; hydrogen; metabisulfites; nitric oxide; nitrites; sulfates such as thiosulfates including but not limited to sodium thiosulfate (Na2S2O3) or calcium thiosulfate (CaS2O3); sulfides such as hydrogen sulfide; sulfites; thionate; thionite; transition metals or their sulfides, oxides, chalcogenides, halides, hydroxides, oxyhydroxides, phosphates, sulfates, or carbonates, in dissolved or solid phases; and conduction or valence band electrons in solid state electrode materials. In some embodiments, said electron acceptors comprise one or more of the following: carbon dioxide; oxygen; nitrites; nitrates; ferric iron or other transition metal ions; sulfates; or valence or conduction band holes in solid state electrode materials.

In some embodiments, the biological conversion step is preceded by one or more chemical preprocessing steps in which said electron donors and/or electron acceptors and/or carbon sources and/or mineral nutrients required by the microorganism, are generated and/or refined from at least one input chemical and/or are recycled from chemicals emerging from the carbon-fixing step and/or are generated from, or are contained within, waste streams from other industrial, mining, agricultural, sewage or waste generating processes.

In some embodiments, the organic chemical product includes compounds with carbon backbones that are five carbons or longer.

In some embodiments, a method is provided for producing amino acids and/or protein and/or vitamins and/or biomass, comprising culturing a microorganism as described herein in a bioreactor that comprises a gaseous substrate and a culture medium that comprises other nutrients for growth and bioproduct production, under conditions that are suitable for growth of the microorganism and production of amino acids and/or protein and/or vitamins and/or biomass, wherein said microorganism produces amino acids and/or protein and/or vitamins and/or biomass.

In some embodiments, at least one chemosynthetic reaction and at least one anabolic biosynthetic pathway results in the formation of biochemicals including at least one of: amino acids; peptides; proteins; lipids; polysaccharides; and/or vitamins.

In some embodiments, biomass and/or biochemicals are produced through the said at least one chemosynthetic reaction, and wherein the biomass and/or biochemicals have application as at least one of the following: as an organic carbon and/or nitrogen source for fermentations; as a nutrient source for the growth of other microbes or organisms; as a nutrient source or food ingredient for humans; as a feed for animals; as a raw material or chemical intermediate for manufacturing or chemical processes; as sources of pharmaceutical, medicinal or nutritional substances; as a fertilizer; as soil additives; and/or as soil stabilizers.

In some embodiments, the carbon and/or nitrogen source from the said chemosynthetic reaction is used in a fermentation to produce biochemicals including at least one of: commercial enzymes, antibiotics, amino acids, protein, food, food ingredients; vitamins, lipids, bioplastics, polysaccharides, neutraceuticals, pharmaceuticals. In some embodiments, said feed for animals is used to feed one or more of: cattle, sheep, chickens, pigs, fish, shellfish, insects, invertebrates, coral. In some embodiments, said shellfish or coral is grown using nutrients biosynthesized from C1 sources, produce carbonate materials that sequester CO₂ into solid mineralized form having high albedo.

In certain embodiments of the present disclosure amino acids, and/or peptides, and/or proteins and/or vitamins are synthesized from simple C1 and inorganic precursors including but not limited to one or more of the following: H₂, CO₂, CO, H₂O, NH₃, CH₄, CH₃OH, HCOH, urea.

In some embodiments, the disclosure relates to a method of producing one or more amino acids or proteins or vitamins, comprising exposing a bacterial cell to syngas and/or producer gas and/or gaseous CO₂ and/or H₂ and/or CO and/or CH₄; wherein the bacterial cell is capable of fixing gaseous CO₂ and/or other C1 molecules into one or more amino acids or proteins or vitamins, and wherein the microorganism comprises zero or at least a first exogenous nucleic acid. In some embodiments, the cell utilizes the said gaseous substrates as a source of reducing equivalents and/or metabolic energy for the synthesis of one or more amino acids or proteins or vitamins. In some embodiments, the microorganism through its native machinery produces amino acids and/or proteins and/or vitamins.

In some embodiments, the disclosure relates to a method for producing amino acids and/or proteins and/or proteinaceous biomass and/or vitamins wherein the method comprises culturing natural strain or an engineered microorganism in a bioreactor or solution with a feedstock comprising syngas and/or producer gas and/or CO₂ and/or H₂ gas and/or CO and/or CH₄. In some embodiments, the disclosure relates to a bioreactor comprising the composition or bacterial or microbial cells described herein. In some embodiments, the disclosure relates to a system for the production of one or more amino acids, proteins, or nutrients, comprising a bioreactor, which comprises: (a) a microorganism population comprising a cell described herein; and (b) an inlet connected to a feedstock source allowing delivery of a feedstock comprising syngas or producer gas and/or gaseous CO₂ and/or H₂ and/or CO and/or CH₄.

In another aspect of the disclosure, the disclosure relates to a method of producing a molecule or mixture of molecules in a microorganism population comprising the cell or the composition described herein, wherein the method comprises: culturing a population of microorganisms comprising the cell or the composition described herein in a feedstock comprising syngas or producer gas and/or gaseous CO₂ and/or H₂ and/or CO and/or CH₄.

In some embodiments the disclosure relates to a method of producing amino acids, or proteins, or other nutrients in a microorganism population comprising the cell of the composition described herein, wherein the method comprises: culturing a population of microorganisms comprising the cell or the composition described herein in a feedstock comprising syngas or producer gas and/or gaseous CO₂ and/or H₂ and/or CO and/or CH₄.

In some embodiments, the disclosure relates to a method of manufacturing one or more amino acids, or proteins, or other nutrients, comprising (a) culturing a cell described herein in a reaction vessel or bioreactor in the presence of syngas or producer gas and/or gaseous CO₂ and/or H₂ and/or CO and/or CH₄, wherein the cell produces and/or secretes one or more amino acids, or proteins, or other nutrients in a quantity equal to or greater than at least 10% of the cell's total dry cellular mass; and (b) separating the one or more amino acids, or proteins, or other nutrients, or a whole cell product from the reaction vessel. In some embodiments, the method further comprises purifying the one or more amino acids, or proteins, or other nutrients, or whole cell products after separation from the reaction vessel or bioreactor. In some embodiments, the one or more amino acids, or proteins, or other nutrients, or whole cell products are components of, or precursors to, or are included within a feed or nutrient supply or fertilizer provided to another organism. In certain non-limiting embodiments that other organism is a heterotroph, and in certain such embodiments an animal including but not limited to one or more of a: zooplankton, shellfish or other invertebrate, fish, bird, or mammal.

In some embodiments, the disclosure relates to a method of producing one or more amino acids comprising exposing a bacterial cell and/or archaeal cell and/or other microbial cell to syngas and/or gaseous CO₂ and/or H₂ and/or CO and/or CH₄; wherein the cell is capable of fixing gaseous CO₂ and/or other C1 carbon sources into one or more amino acids and/or proteins and/or vitamins; wherein the compounds are recovered from the bioreactor and fed to a second or more additional reactors and/or process steps wherein the compounds are post-processed to generate products including but not limited to one or more of the following: fertilizer, aquaculture feed, animal feed, human nutrition, or vitamins.

In some embodiments the present disclosure gives compositions and methods for the capture of carbon dioxide from carbon dioxide-containing gas streams and/or atmospheric carbon dioxide or carbon dioxide in dissolved, liquefied or chemically-bound form through a chemical and biological process that utilizes obligate or facultative chemoautotrophic microorganisms and particularly chemolithoautotrophic organisms, and/or cell extracts containing enzymes from chemoautotrophic microorganisms in one or more carbon fixing process steps. The present disclosure also gives compositions and methods for the recovery, processing, and use of the chemical products of chemosynthetic reactions performed by chemoautotrophs to fix inorganic carbon into organic compounds that are intermediate or finished chemicals, including but not limited to amino acids and/or protein and/or vitamins and/or biomass. The present disclosure also gives compositions and methods for the generation, processing and delivery of chemical nutrients needed for chemosynthesis and maintenance of chemoautotrophic cultures, including but not limited to the provision of electron donors and electron acceptors needed for chemosynthesis. The present disclosure also gives compositions and methods for the maintenance of an environment conducive for chemosynthesis and chemoautotrophic growth, and the recovery and recycling of unused chemical nutrients and process water.

In some embodiments, the microorganisms disclosed herein are recombinantly engineered to express one or more enzymes for biosynthetic production of amino acids, proteins, and other biological nutrients. In some embodiments, substrates or intermediates are diverted to the synthesis of amino acids, proteins, and/or other biological nutrients in the microbial cells, for example, acetyl-CoA, pyruvate, or malonyl-CoA. In some non-limiting embodiments, some fraction of carbon flux along the various biosynthesis pathways is directed into the biosynthesis of targeted amino acids, proteins, and other biological nutrients.

One feature of certain embodiments of the present disclosure is the inclusion of one or more process steps that utilize chemotrophic microorganisms and/or enzymes from chemotrophic microorganisms as a biocatalyst for the conversion of C1 chemicals into longer carbon chain organic molecules (i.e., C2 or longer and, in some embodiments, C5 or longer carbon chain molecules), within an overall process for the conversion of C1 carbon sources including but not limited to carbon monoxide, methane, methanol, formate, or formic acid, and/or mixtures containing C1 chemicals including but not limited to various syngas compositions generated from various gasified, pyrolyzed, or steam-reformed fixed carbon feedstocks and/or methane feedstocks. In some such embodiments C1 containing syngas, or process gas, or C1 chemicals in a liquid form or dissolved in solution are pumped or otherwise added to a vessel or enclosure containing nutrient media and chemotrophic microorganisms. In some such cases chemotrophic microorganisms perform biochemical synthesis to elongate C1 chemicals into longer carbon chain organic chemicals using the carbon and electrons stored in the C1 chemical, and/or electrons and hydrogen from molecular hydrogen and/or valence or conduction electrons in solid state electrode materials and/or one or more of the following list of electron donors pumped or otherwise provided to the nutrient media, which include, but are not limited to one or more of the following: ammonia; ammonium; carbon monoxide; dithionite; elemental sulfur; hydrocarbons; metabisulfites; nitric oxide; nitrites; sulfates such as thiosulfates including but not limited to sodium thiosulfate (Na₂S₂O₃) or calcium thiosulfate (CaS₂O₃); sulfides such as hydrogen sulfide; sulfites; thionate; thionite; transition metals or their sulfides, oxides, chalcogenides, halides, hydroxides, oxyhydroxides, sulfates, or carbonates, in soluble or solid phases. The electron donors can be oxidized by electron acceptors in a chemosynthetic respiratory reaction. In certain embodiments, electron acceptors that are used for respiration by the microorganisms of the present disclosure include but are not limited to one or more of the following: oxygen, carbon dioxide, ferric iron or other transition metal ions, nitrates, nitrites, oxygen, or holes in solid state electrode materials. In certain non-limiting embodiments, the said chemotrophic microorganism is a knallgas or oxyhydrogen microorganism.

In certain embodiments the disclosure relates to chemotrophic bacterial strains that comprise zero or more exogenous nucleic acid sequences. The present disclosure arises in part from the discovery that chemotrophic bacteria and particular related microorganisms provide unforeseen advantages in the economic and large scale production of chemicals, proteins, feeds, fertilizers, monomers, oils, fuels, and other biological substances from gaseous and waste carbon feedstocks, and also from the discovery of genetic techniques and systems for modifying these microorganisms for improved performance in these applications. The proteins, lipids and other biochemicals synthesized by the microorganisms of the present disclosure can be applied to uses including but not limited to petrochemical substitutes, monomers, feedstock for the production of polymers, lubricants, as ingredients in fertilizer, animal feed, food, personal care, and cosmetic products. In some embodiments of the present disclosure enzymatic and chemical processes can be utilized to produce vitamins, amino acids, and/or proteins. Some embodiments enable the production of animal feeds and/or fertilizers. In addition, the present disclosure gives methods for culturing and/or modifying chemotrophic bacteria for improved amino acid and/or protein yield and/or lower production costs. In some embodiments, a genetically modified bacterium produces more of a certain type or types of vitamin or amino acid molecules as compared to the same bacteria that is not genetically modified.

The disclosure relates to methods and mechanisms to confer production and/or secretion of carbon-based products of interest including but not limited to chemicals, monomers, polymers, amino acids, proteins, polysaccharides, vitamins, nutraceutical or pharmaceutical products or intermediates thereof in obligate or facultative chemotrophic organisms such that these organisms convert carbon dioxide and/or other forms of inorganic carbon and/or syngas and/or other C1 compounds such as methanol and/or the liquid, gaseous, and solid products of pyrolytic reactions such as pyrolysis gas and/or oil, into carbon-based products of interest, and in particular the use of such organisms for the commercial production of chemicals, monomers, polymers, amino acids, proteins, polysaccharides, vitamins, animal feeds, fertilizers, nutraceutical or pharmaceutical products or intermediates thereof.

In some embodiments the present disclosure also gives compositions and methods for chemical process steps that occur in series and/or in parallel with the chemosynthetic reaction steps that: convert unrefined raw input chemicals to more refined chemicals that are suited for supporting the chemosynthetic carbon fixing step; that convert energy inputs into a chemical form that can be used to drive chemosynthesis, and specifically into chemical energy in the form of electron donors and electron acceptors; that direct inorganic carbon captured from industrial or atmospheric or aquatic sources to the carbon fixation step or steps of the process under conditions that are suitable to support chemosynthetic carbon fixation; that further process the output products of the chemosynthetic carbon fixation steps into a form suitable for storage, shipping, and sale, with said products including but not limited to amino acids and/or proteins and/or vitamins and/or biomass. The fully chemical, abiotic, process steps combined with the biological chemosynthetic carbon fixation steps constitute the overall carbon capture and conversion process of the present disclosure. The present disclosure utilizes the unique ease of integrating chemoautotrophic microorganisms within a chemical process stream as a biocatalyst, as compared to other lifeforms. While not intending to be limited by theory, this unique capability and facility appears to arise from the fact that chemoautotrophs naturally act at the interface of biology and abiotic chemistry through their chemosynthetic mode of existence.

In some embodiments the natural or engineered strain includes but is not limited to Corynebacterium autotrophicum. In some embodiments, the natural or engineered strain includes but is not limited to Corynebacterium glutamicum. In some embodiments, the microorganism is Hydrogenovibrio marinus. In some embodiments, the microorganism is Rhodopseudomonas capsulata, Rhodopseudomonas palustris, or Rhodobacter sphaeroides.

In some embodiments, the microorganism is an oxyhydrogen or knallgas strain. In some embodiments the microorganisms comprise one or more of the following knallgas microorganisms: Aquifex pyrophilus, Aquifex aeolicus, or other Aquifex sp.; Cupriavidus necator, Cupriavidus metallidurans, or other Cupriavidus sp.; Corynebacterium autotrophicum or other Corynebacterium sp.; Gordonia desulfuricans, Gordonia polyisoprenivorans, Gordonia rubripertincta, Gordonia hydrophobica, Gordonia westfalica, and other Gordonia sp.; Nocardia autotrophica, Nocardia opaca, or other Nocardia sp.; purple non-sulfur photosynthetic bacteria including but not limited to Rhodobacter sphaeroides, Rhodopseudomonas palustris, Rhodopseudomonas capsulata, Rhodopseudomonas viridis, Rhodopseudomonas sulfoviridis, Rhodopseudomonas blastica, Rhodopseudomonas spheroides, Rhodopseudomonas acidophila and other Rhodopseudomonas sp. and Rhodobacter sp.; Rhodospirillum rubrum, and other Rhodospirillum sp.; Rhodococcus opacus and other Rhodococcus sp.; Rhizobium japonicum and other Rhizobium sp.; Thiocapsa roseopersicina and other Thiocapsa sp.; Pseudomonas facilis, Pseudomonas flava, Pseudomonas putida, Pseudomonas hydrogenovora, Pseudomonas hydrogenothermophila, Pseudomonas palleronii, Pseudomonas pseudoflava, Pseudomonas saccharophila, Pseudomonas thermophile, and other Pseudomonas sp.; Hydrogenomonas pantotropha, Hydrogenomonas eutropha, Hydrogenomonas facilis, and other Hydrogenomonas sp.; Hydrogenobacter thermophiles, Hydrogenobacter halophilus, Hydrogenobacter hydrogenophilus, and other Hydrogenobacter sp.; Hydrogenophilus islandicus and other Hydrogenophilus sp.; Hydrogenovibrio marinus and other Hydrogenovibrio sp.; Hydrogenothermus marinus and other Hydrogenothermus sp.; Helicobacter pylori and other Helicobacter sp.; Xanthobacter autotrophicus, Xanthobacter flavus and other Xanthobacter sp.; Hydrogenophaga flava, Hydrogenophaga palleronii, Hydrogenophaga pseudoflava and other Hydrogenophaga sp.; Bradyrhizobium japonicum and other Bradyrhizobium sp.; Ralstonia eutropha and other Ralstonia sp.; Alcaligenes eutrophus, Alcaligenes facilis, Alcaligenes hydrogenophilus, Alcaligenes latus, Alcaligenes paradoxus, Alcaligenes ruhlandii and other Alcaligenes sp.; Amycolata sp.; Aquaspirillum autotrophicum and other Aquaspirillum sp.; Arthrobacter strain 11/X, Arthrobacter methylotrophus, and other Arthrobacter sp.; Azospirillum lipoferum and other Azospirillum sp.; Variovorax paradoxus, and other Variovorax sp.; Acidovorax facilis, and other Acidovorax sp.; Bacillus schlegelii, Bacillus tusciae and other Bacillus sp.; Calderobacterium hydrogenophilum and other Calderobacterium sp.; Derxia gummosa and other Derxia sp.; Flavobacterium autothermophilum and other Flavobacterium sp.; Microcyclus aquaticus and other Microcyclus; Mycobacterium gordoniae and other Mycobacterium sp.; Paracoccus denitrificans and other Paracoccus sp.; Persephonella marina, Persephonella guaymasensis and other Persephonella sp.; Renobacter vacuolatum and other Renobacter sp.; Streptomycetes coelicoflavus, Streptomycetes griseus, Streptomycetes xanthochromogenes, Streptomycetes thermocarboxydus, and other Streptomycetes sp.; Thermocrinis ruber and other Thermocrinis sp.; Wautersia sp.; cyanobacteria including but not limited to Anabaena oscillarioides, Anabaena spiroides, Anabaena cylindrica, and other Anabaena sp., and Arthrospira platensis, Arthrospira maxima and other Arthrospira sp.; green algae including but not limited to Scenedesmus obliquus and other Scenedesmus sp., Chlamydomonas reinhardii and other Chlamydomonas sp., Ankistrodesmus sp., Rhaphidium polymorphium and other Rhaphidium sp; as well as a consortiums of microorganisms that include oxyhydrogen microorganisms.

In some non-limiting embodiments the disclosure relates to compositions comprising and methods of using chemoautotrophic metabolism to produce ATP for the support of ATP consuming biosynthetic reactions and cellular maintenance, without the co-production of methane or short chain organic acids such as acetic or butyric acid, by means of energy conserving reactions for the production of ATP, which use inorganic electron donors and electron acceptors, including but not limited to the oxyhydrogen reaction.

A number of different microorganisms have been characterized that are capable of growing on carbon monoxide as an electron donor and/or carbon source (i.e. carboxydotrophic microorganisms). In some cases, carboxydotrophic microorganisms can also use H₂ as an electron donor and/or grow mixotrophically. In some cases, the carboxydotrophic microorganisms are facultative chemolithoautotrophs. In some embodiments the microorganisms comprise one or more of the following carboxydotrophic microorganisms: Acinetobacter sp.; Alcaligenes carboxydus and other Alcaligenes sp.; Arthrobacter sp.; Azomonas sp.; Azotobacter sp.; Bacillus schlegelii and other Bacillus sp.; Hydrogenophaga pseudoflava and other Hydrogenophaga sp.; Pseudomonas carboxydohydrogena, Pseudomonas carboxydovorans, Pseudomonas compransoris, Pseudomonas gazotropha, Pseudomonas thermocarboxydovorans and other Pseudomonas sp.; Rhizobium japonicum and other Rhizobium sp.; Streptomyces G26 Streptomyces thermoautotrophicus and other Streptomyces sp. In certain embodiments of the present disclosure a carboxydotrophic microorganism is used. In certain embodiments, a carboxydotrophic microorganism that is capable of chemolithoautotrophy is used. In certain embodiments, a carboxydotrophic microorganism that is able to use H2 as an electron donor in respiration and/or biosynthesis is used.

In some embodiments the microorganisms comprise obligate and/or facultative chemoautotrophic microorganisms including one or more of the following: Acetoanaerobium sp.; Acetobacterium sp.; Acetogenium sp.; Achromobacter sp.; Acidianus sp.; Acinetobacter sp.; Actinomadura sp.; Aeromonas sp.; Alcaligenes sp.; Alcaligenes sp.; Aquaspirillum sp.; Arcobacter sp.; Aureobacterium sp.; Bacillus sp.; Beggiatoa sp.; Butyribacterium sp.; Carboxydothermus sp.; Clostridium sp.; Comamonas sp.; Dehalobacter sp.; Dehalococcoide sp.; Dehalospirillum sp.; Desulfobacterium sp.; Desulfomonile sp.; Desulfotomaculum sp.; Desulfovibrio sp.; Desulfurosarcina sp.; Ectothiorhodospira sp.; Enterobacter sp.; Eubacterium sp.; Ferroplasma sp.; Halothibacillus sp.; Hydrogenobacter sp.; Hydrogenomonas sp.; Leptospirillum sp.; Metallosphaera sp.; Methanobacterium sp.; Methanobrevibacter sp.; Methanococcus sp.; Methanococcoides sp.; Methanogenium sp.; Methanolobus sp.; Methanomicrobium sp.; Methanoplanus sp.; Methanosarcina sp.; Methanospirillum sp.; Methanothermus sp.; Methanothrix sp.; Micrococcus sp.; Nitrobacter sp.; Nitrobacteraceae sp., Nitrococcus sp., Nitrosococcus sp.; Nitrospina sp., Nitrospira sp., Nitrosolobus sp.; Nitrosomonas sp.; Nitrosospira sp.; Nitrosovibrio sp.; Nitrospina sp.; Oleomonas sp.; Paracoccus sp.; Peptostreptococcus sp.; Planctomycetes sp.; Pseudomonas sp.; Ralstonia sp.; Rhodobacter sp.; Rhodococcus sp.; Rhodocyclus sp.; Rhodomicrobium sp.; Rhodopseudomonas sp.; Rhodospirillum sp.; Shewanella sp.; Siderococcus sp.; Streptomyces sp.; Sulfobacillus sp.; Sulfolobus sp.; Thermothrix sp., Thiobacillus sp.; Thiomicrospira sp.; Thioploca sp.; Thiosphaera sp.; Thiothrix sp.; Thiovulum sp.; sulfur-oxidizers; hydrogen-oxidizers; iron-oxidizers; acetogens; and methanogens; consortiums of microorganisms that include chemoautotrophs; chemoautotrophs native to at least one of hydrothermal vents, geothermal vents, hot springs, cold seeps, underground aquifers, salt lakes, saline formations, mines, acid mine drainage, mine tailings, oil wells, refinery wastewater. coal seams, deep sub-surface; waste water and sewage treatment plants; geothermal power plants, sulfatara fields, and soils; and extremophiles selected from one or more of thermophiles, hyperthermophiles, acidophiles, halophiles, and psychrophiles.

Such organisms also include but are not limited to extremophiles that can withstand extremes in various environmental parameters such as temperature, radiation, pressure, gravity, vacuum, desiccation, salinity, pH, oxygen tension, and chemicals. They include hyperthermophiles, such as Pyrolobusfumarii; thermophiles, such as Synechococcus lividis; mesophiles, and psychrophiles, such as Psychrobacter. Extremely thermophilic sulfur-metabolizers such as Thermoproteus sp., Pyrodictium sp., Sulfolobus sp., Acidianus sp. Radiation tolerant organisms include Deinococcus radiodurans. Pressure tolerant organisms include piezophiles or barophiles. Desiccant tolerant and anhydrobiotic organisms include xerophiles; microbes and fungi. Salt tolerant organisms include halophiles, such as Halobacteriacea and Dunaliella salina. pH tolerant organisms include alkaliphiles such as Natronobacterium, Bacillus firmus OF4, Spirulina spp., and acidophiles such as Cyanidium caldarium, Ferroplasma sp. Gas tolerant organisms, which tolerate pure CO2 include Cyanidium caldarium and metal tolerant organisms include metalotolerants such as Ferroplasma acidarmanus, Ralstonia sp.

In some embodiments, the disclosure further provides a composition wherein the microorganism is a hydrogen-oxidizing chemoautotroph and/or a carboxydotroph and/or a methylotroph and/or methanotroph. In some embodiments, the disclosure further provides a composition wherein the microorganism is capable of growing on syngas and/or producer gas and/or pyrolysis gas as the sole electron donor, and/or source of reduced hydrogen atoms, and/or carbon source. In some embodiments, the disclosure further provides a composition wherein the microorganism is capable of growing on untreated crude glycerol as the sole electron donor, and/or source of reduced hydrogen atoms, and/or carbon source.

In certain embodiments of the present disclosure the microbes used are naturally occurring and/or non-genetically modified (non-GMO) microorganisms and/or non-pathogenic and/or rely on specific environmental conditions provided by the bioprocesses that are absent from the surrounding environment.

Certain embodiments of the present disclosure utilize a microorganism or consortium of microorganisms, isolated from environmental samples and enriched with desirable microorganisms using methods known in the art of microbiology through growth in the presence of targeted electron donors including but not limited to one or more of: hydrogen and/or CO and/or syngas and/or methane, and electron acceptors including but not limited to one or more of oxygen and/or nitrate and/or ferric iron and/or CO₂, and environmental conditions (e.g. temperature, pH, pressure, DO, salinity, the presence of various impurities and pollutants etc.).

In some embodiments, the disclosure further provides a method wherein the electron donors utilized in biosynthesis and/or respiration include but are not limited to one or more of the following reducing agents: ammonia; ammonium; carbon monoxide; dithionite; elemental sulfur; hydrogen; metabisulfites; nitric oxide; nitrites; sulfates such as thiosulfates including but not limited to sodium thiosulfate (Na₂S₂O₃) or calcium thiosulfate (CaS₂O₃); sulfides such as hydrogen sulfide; sulfites; thionate; thionite.

In some embodiments the microorganism is a methanotroph. In some embodiments, the microorganism is in the genus Methylococcus. In some embodiments, the microorganism is Methylococcus capsulatus. In some embodiments, the microorganism is a methylotroph. In some embodiments, the microorganism is in the genus Methylobacterium. In some embodiments, the microorganism is drawn from one or more of the following species: Methylobacterium zatmanii; Methylobacterium extorquens; Methylobacterium chloromethanicum.

In some embodiments the microorganism of the claimed disclosure is not dependent upon light to grow and/or to synthesize one or more of the following: amino acids and/or proteins and/or other nutrients. In some embodiments, the microorganism of the claimed disclosure does not require any type of sugar or any other type of organic compound or any type of fixed carbon to grow and/or to synthesize one or more of the following: amino acids and/or proteins and/or other nutrients. In some embodiments, the microorganism of the claimed disclosure is a facultative microorganism.

The production of organic molecules with carbon chain lengths longer than C4 is most commonly and efficiently accomplished biologically through anabolic biosynthesis pathways such as fatty acid biosynthesis, and various amino acid biosynthetic pathways. The initial molecule entering into the fatty acid biosynthesis pathway is acetyl-coenzyme A (acetyl-CoA), a central metabolite from which many high value biochemicals can be derived. In some embodiments, the disclosure utilizes microorganisms with a naturally occurring pathway for the conversion of CO, CO₂ and/or H₂ and/or CH₄ to acetyl-CoA. In some embodiments, the disclosure utilizes microorganisms that can fix CO and/or CO₂ through the reductive tricarboxylic acid cycle, the Calvin-Benson-Bassham cycle, and/or the Wood-Ljungdahl pathway. In some embodiments, the disclosure utilizes microorganisms the fix C1 compounds through a methanotrophic pathway. In some embodiments the microorganisms naturally produce enzymes that catalyze the fixation of gaseous inorganic carbon to produce one or more of acetyl-CoA, pyruvate, malonyl-CoA, utilizing gaseous electron donors such as are present in syngas and/or producer gas as reducing agents, with such enzymatic proteins including but not limited to acetyl-CoA synthase, acetyl-CoA synthase disulfide reductase, cobalamide corrinoid/iron-sulfur protein, carbon monoxide dehydrogenase, hydrogenase, and methyltransferase.

In some embodiments, the disclosure relates to a microorganism or compositions comprising a microorganism, wherein the microorganism is able to produce ATP from an inorganic electron donor such as but not limited to H₂ and/or CO without the synthesis of methane or short chain organic acids (short chain organic acids comprising carbon chain lengths from two to four carbons long). In some non-limiting embodiments, the disclosure relates to a microorganism or compositions comprising a microorganism, wherein the microorganism produces ATP from an inorganic electron donor such as but not limited to H₂ and/or CO, coupled with an electron acceptor other than CO₂ that is used in respiration.

Certain embodiments of the present disclosure apply hydrogen-oxidizing and/or CO-oxidizing and/or CH₄ oxidizing microorganisms that use more electronegative electron acceptors in energy conserving reactions for ATP production, such as but not limited to O₂. For example, hydrogenotrophic oxyhydrogen or knallgas microbes that couple the oxyhydrogen reaction, 2H₂+O₂->2H₂O, to ATP production, can produce more ATP per H₂ and/or other electron donor consumed for respiration, than acetogens or methanogens that use CO₂ as an electron acceptor in respiration. For example, knallgas microorganisms can produce at least two ATP per H₂ consumed in respiration, which is eight times more ATP produced per H₂ consumed in respiration than what can be produced in microorganisms undergoing methanogenesis or acetogenesis, using H₂ as electron donor and CO₂ as electron acceptor in respiration.

In certain embodiments, the oxyhydrogen reaction used in respiration is enzymatically linked to oxidative phosphorylation. In certain embodiments, the ATP and/or other intracellular energy carriers thus formed are utilized in the anabolic synthesis of amino acids and/or proteins. In some embodiments, the disclosure relates to a knallgas microorganism or compositions comprising a knallgas microorganism, wherein the microorganism comprises at least zero or one or more exogenous nucleic acid sequences that encodes zero or more enzymes to enable biosynthesis of useful carbon-based products of interest including but not limited to chemicals, monomers, polymers, proteins, polysaccharides, vitamins, nutraceuticals, antibiotics, or pharmaceutical products or intermediates thereof from a carbon-containing gas feedstock, including but not limited to syngas or producer gas or waste CO₂ combined with renewable H₂ or CO or methane containing gases. In some non-limiting embodiments, the disclosure relates to a microorganism or compositions comprising a microorganism, wherein the microorganism requires less than 4H₂ to produce one ATP through respiration. In other non-limiting embodiments, the disclosure relates to a microorganism or compositions comprising a microorganism, wherein the microorganism produces more than one ATP per H₂ consumed through respiration. In other non-limiting embodiments, the disclosure relates to a microorganism or compositions comprising a microorganism, wherein the microorganism produces at least two ATP per H₂ consumed through respiration, or at least 2.5 ATP per H₂ consumed through respiration.

In some embodiments, the disclosure relates to a composition comprising a microorganism that converts syngas and/or producer gas and/or gaseous CO₂ and/or H₂ and/or CO and/or CH₄ into one or more organic compounds, wherein less than 10% by weight of the organic compounds produced by the microorganism is methane. In some embodiments, the disclosure relates to a composition comprising a microorganism that converts said gaseous substrates into one or more organic compounds; wherein less than 10% by weight of the organic compounds produced are free organic acids with carbon chain length of four carbons or less.

In certain embodiments of the present disclosure the microorganism reduces CO₂, producing cell material and H₂O. In certain embodiments, the energy needed for the metabolic pathways that perform this reduction is obtained by the oxidation of hydrogen with molecular oxygen. In certain embodiments of the present disclosure the biological system and/or components function directly as a CO₂ reducer, but not an O₂ producer. In certain embodiments, the O₂ utilized in respiration is obtained from another system and provided to the biological system and/or components. In certain embodiments that other system involves the electrolysis and/or thermolysis of water.

For some embodiments gases that may be pumped into a bioreactor include but not are not limited to one or more of the following: syngas, producer gas, pyrolysis gas, hydrogen gas, CO, CO₂, O₂, air, air/CO₂ mixtures, natural gas, biogas, methane, ammonia, nitrogen, noble gases, such as argon, as well as other gases. In some embodiments the CO₂ pumped into the system may come from sources including but are not limited to: CO₂ from the gasification of organic matter; CO₂ from the calcination of limestone, CaCO₃, to produce quicklime, CaO; CO₂ from methane steam reforming, such as the CO₂ byproduct from ammonia, methanol, or hydrogen production; CO₂ from combustion, incineration, or flaring; CO₂ byproduct of anaerobic or aerobic fermentation of sugar; CO₂ byproduct of a methanotrophic bioprocess; CO₂ from waste water treatment; CO₂ byproduct from sodium phosphate production; geologically or geothermally produced or emitted CO₂; CO₂ removed from acid gas or natural gas. In certain embodiments, the carbon source is CO₂ and/or bicarbonate and/or carbonate in sea water or other bodies of surface or underground water. In certain embodiments, the carbon source is CO₂ from the atmosphere. In certain non-limiting embodiments, the CO₂ has been captured from a closed cabin as part of a closed-loop life support system, using equipment such as but not limited to a CO₂ removal assembly (CDRA), which is utilized on the International Space Station (ISS).

In certain embodiments of the present disclosure, carbon dioxide containing flue gases are captured from a smoke stack at temperature, pressure, and gas composition characteristic of the untreated exhaust, and directed with minimal modification into the reaction vessels where carbon-fixation occurs. In some embodiments in which impurities harmful to organisms are not present in the flue gas, modification of the flue gas upon entering the reaction vessels can be limited to the compression needed to pump the gas through the reactor system and/or the heat exchange needed to lower the gas temperature to one suitable for exposure to the microorganisms. In certain embodiments, the CO₂ present in a flue gas or other mixed gas stream is purified and/or concentrated prior to introduction into the bioreactor using carbon-capture technologies and processes well known in the art.

In embodiments in which carbon dioxide bearing flue gas is transported through a system for dissolving the carbon dioxide into solution (such as is well known in the art of carbon capture and/or microbial conversion), the scrubbed flue gas with reduced CO₂ content, (which generally primarily includes inert gases such as nitrogen), can in certain embodiments be released into the atmosphere.

In certain embodiments of the present disclosure the carbon source is CO2 and/or CO contained in industrial flue or off-gases and/or from natural sources including but not limited to geological and geothermal sources. In certain embodiments, the CO2 and/or CO containing flue and/or off gases utilized are emitted from one or more of the following industries or sectors: oil; electricity; natural gas; cement; chemicals; steel; metallurgy; fermentation; waste water treatment. In certain non-limiting embodiments of the present disclosure a relatively small land-footprint, facilitates collocation of the bioprocess with industrial facilities producing CO₂ and/or other carbon wastes including but not limited to one or more of the following: fossil power plants; oil refineries; tar sands upgrading facilities; natural gas or petroleum drilling operations; ethanol distilleries; cement manufactures; aluminum manufactures, chloroalkali manufactures, steel foundries; geothermal power plants. In certain embodiments of the present disclosure waste-heat associated with industrial flue-gas sources is further utilized in the production process of the present disclosure for steps including but not limited to in biomass drying.

In certain embodiments gases in addition to carbon dioxide, or in place of carbon dioxide as an alternative carbon source, are either dissolved into solution and fed to the culture broth and/or dissolved directly into the culture broth including but not limited to gaseous electron donors and/or carbon sources (e.g., hydrogen and/or CO and/or methane gas). In certain embodiments of the present disclosure, input gases may include other electron donors and/or electron acceptors and/or carbon sources and/or mineral nutrients such as but not limited to other gas constituents and impurities of syngas (e.g., hydrocarbons); ammonia; hydrogen sulfide; and/or other sour gases; and/or O₂; and/or mineral containing particulates and ash.

In certain embodiments of the present disclosure gases are dissolved into the culture broth of the present disclosure including but not limited to gaseous electron donors such as but not limited to one or more of the following: hydrogen, carbon monoxide, methane, hydrogen sulfide or other sour gases; gaseous carbon sources such as but not limited to one or more of the following CO₂, CO, CH₄; and electron acceptors such as but not limited to oxygen, either within air (e.g. 20.9% oxygen) or as pure O₂ or as an O₂-enriched gas. In some embodiments, the dissolution of these and other gases into solution is achieved using a system of compressors, flowmeters, and flow valves known to one skilled in the art of fermentation engineering, that feed into one of more of the following widely used systems for dispersing gas into solution: sparging equipment; diffusers including but not limited to dome, tubular, disc, or doughnut geometries; coarse or fine bubble aerators; venturi equipment. In certain embodiments of the present disclosure surface aeration and/or gas mass transfer may also be performed using paddle aerators and the like. In certain embodiments of the present disclosure gas dissolution is enhanced by mechanical mixing with an impeller or turbine, as well as hydraulic shear devices to reduce bubble size. Following passage through the reactor system holding microorganisms which uptake the gases, in certain embodiments the residual gases may either be recirculated back to the bioreactor, or burned for process heat, or flared, or injected underground, or released into the atmosphere. In certain embodiments of the present disclosure utilizing H₂ as electron donor, H₂ may be fed to the culture vessel either by bubbling it through the culture medium, or by diffusing it through a hydrogen permeable-water impermeable membrane known in the art that interfaces with the liquid culture medium.

In certain embodiments the microorganisms grow and multiply on the H₂ and CO₂ and other dissolved nutrients under microaerobic conditions. In certain embodiments a C1 chemical such as but not limited to carbon monoxide, methane, methanol, formate, or formic acid, and/or mixtures containing C1 chemicals including but not limited to various syngas compositions generated from various gasified, pyrolyzed, or steam-reformed fixed carbon feedstocks, are biochemically converted into longer chain organic chemicals (i.e. C₂ or longer and, in some embodiments, C5 or longer carbon chain molecules) under one or more of the following conditions: aerobic, microaerobic, anoxic, anaerobic, and/or facultative conditions.

A controlled amount of oxygen can also be maintained in the culture broth of some embodiments of the present disclosure, and in certain embodiments, oxygen will be actively dissolved into solution fed to the culture broth and/or directly dissolved into the culture broth. In certain aerobic or microaerobic embodiments of the present disclosure that require the pumping of air or oxygen into the culture broth in order to maintain targeted DO levels, oxygen bubbles may be injected into the broth at an optimal diameter for mixing and oxygen transfer. In certain aerobic embodiments of the present disclosure a process of shearing the oxygen bubbles may be used to achieve this bubble diameter as described in U.S. Pat. No. 7,332,077. In certain embodiments bubbles, larger than 7.5 mm average diameter and/or slugging are avoided.

In some embodiments, the inventive subject matter converts a fuel gas including but not limited to syngas, producer gas, pyrolysis gas, biogas, tailgas, fluegas, CO, CO₂, H₂, and mixtures thereof. In some embodiments, the heat content of the fuel gas is at least 100 BTU per standard cubic foot (scf). In some embodiments of the present disclosure, a bioreactor is used to contain and grow the microorganisms, which is equipped with fine-bubble diffusers and/or high-shear impellers for gas delivery.

In some embodiments oxygen is used as an electron acceptor in the respiration of the microorganism used for the biosynthesis of amino acids, or proteins, or other nutrients, or whole cell products. In some embodiments, strong electron acceptors including but not limited to O₂ are used to maximize efficiency and yield of products produced via anabolic pathways such as amino acids, fatty acids, or vitamins. A key challenge with using O₂ as an electron acceptor is keeping O₂ levels sufficiently adequate to allow aerobic microbes to grow well and efficiently generate anabolic products while also maintaining appropriate and safe levels of inflammable H₂ and O₂ mixtures, as well as other fuel gas/O₂ mixtures, in the bioreactor to minimize the risk of explosion. In some embodiments, custom or specialized reactor designs are used to control O₂ in the broth at a level that is optimal for the microbes while avoiding dangerous gas mixes. In some embodiments bioreactor designs are used that avoid dangerous mixtures of H₂ and O₂, while providing the microorganisms with necessary levels of these gases for cellular energy, carbon fixation, and for the production of amino acid, or protein, or other nutrients, or whole cells.

Introducing and/or raising the gas flow rate into a bioreactor can enhance mixing of the culture and produce turbulence if the gas inlet is positioned beneath the surface of the liquid media such that gas bubbles or sparges up through the media. In certain embodiments mixing is enhanced through turbulence provided by gas bubbles and/or sparging and/or gas plugging up through the liquid media. In some embodiments, a bioreactor comprises gas outlet ports for gas escape and pressure release. In some embodiments, gas inlets and outlets are preferably equipped with check valves to prevent gas backflow.

In certain embodiments where chemosynthetic reactions occur within the bioreactor, one or more types of electron donor and one or more types of electron acceptor are pumped or otherwise added as either a bolus addition, or periodically, or continuously to the nutrient medium containing chemoautotrophic organisms in the reaction vessel. The chemosynthetic reaction driven by the transfer of electrons from electron donor to electron acceptor in cellular respiration fixes inorganic carbon dioxide and/or other dissolved carbonates and/or other carbon oxides into organic compounds and biomass.

In certain embodiments a nutrient media for culture growth and production is used comprising an aqueous solution containing suitable minerals, salts, vitamins, cofactors, buffers, and other components needed for microbial growth, known to those skilled in the art.

In some embodiments the biomass produced through the present disclosure is converted to animal feed or incorporated into an animal feed formulation or utilized as a source of human nutrition.

To assist in the processing of the biomass product into useful products, harvested microbial cells in certain embodiments of the disclosure can be broken open using well known methods including but not limited to one or more of the following: ball milling, cavitation pressure, sonication, homogenization, or mechanical shearing.

The harvested biomass in some embodiments may be dried in a process step or steps. Biomass drying can be performed in certain embodiments of the present disclosure using well known technologies including but not limited to one or more of the following: centrifugation, drum drying, evaporation, freeze drying, heating, spray drying, vacuum drying, and/or vacuum filtration. In certain embodiments of the present disclosure waste heat can be used in drying the biomass. In certain embodiments heat waste from the industrial source of flue gas used as a carbon source can be used in drying the biomass. In certain embodiments, the heat co-product from the generation of electron donors and/or C1 carbon source as discussed above can be used for drying the biomass.

In certain embodiments of the disclosure, the biomass is further processed following drying, or, without a preceding drying step, in order to aid the separation and production of useful biochemicals. In certain embodiments, this additional processing involves the separation of the protein or lipid content or vitamins or other targeted biochemicals from the microbial biomass. In certain embodiments, the separation of the lipids can be performed by using nonpolar solvents to extract the lipids such as, but not limited to one or more of: hexane, cyclohexane, ethyl ether, alcohol (isopropanol, ethanol, etc.), tributyl phosphate, supercritical carbon dioxide, trioctylphosphine oxide, secondary and tertiary amines, or propane. In certain embodiments, other useful biochemicals can be extracted using solvents including but not limited to one or more of: chloroform, acetone, ethyl acetate, and tetrachloroethylene.

In some embodiments, the instant disclosure provides for a method of producing amino acids and/or proteins by combining, in a bioreactor or solution, one or more biosynthetic pathways including but not limited to an amino acid biosynthetic pathway, a carbon-containing gas, and an engineered or natural microorganism that converts a carbon-containing gas such as syngas, producer gas, CO₂, carbon monoxide and mixtures of the same containing hydrogen gas; and/or C1 compounds, gaseous or liquid, including but not limited to methanol or methane, into amino acids and/or proteins. In some embodiments, the amino acids and/or proteins are included in an animal feed formulation using processes known in the art and science of chemistry, chemical engineering, and food science.

In certain embodiments of the present disclosure proteinaceous biomass produced through the disclosure is used as an alternative protein source. In certain embodiments, it is used as a replacement for fish meal or casein or whey or soy meal. In certain embodiments of the present disclosure proteins produced according to the disclosure are used in feed or fertilizer formulations in place of fish meal or casein or whey or soy meal or other plant proteins. In certain non-limiting embodiments of the present disclosure the protein products are not deficient in any essential amino acids. In certain non-limiting embodiments, the protein products are not deficient in lysine and/or methionine. In certain non-limiting embodiments, the proteinaceous biomass does not contain significant amounts of anti-nutritional factors. In certain embodiments, the proteinaceous biomass does not contain significant amounts of one or more of the following: gossypol, glucosinolates, saponins, trypsin inhibitors. In certain embodiments, the proteinaceous biomass serves as a non-conventional protein source that is suitable for species including but not limited to Oreochromis niloticus.

In one embodiment, the systems disclosed herein relate to generating fine bubbles and may include a vessel containing a liquid, a plate comprising a plurality of orifices positioned in an upper portion of the vessel and configured to accelerate at least a portion of the liquid in the vessel, and at least one sparger positioned within the vessel with a surface of the sparger positioned from about 50 mm to about 300 mm, 500 mm, or 1000 mm from a bottom of the plate. The sparger may be configured to inject bubbles into the liquid. In some examples, the sparger may be positioned within the vessel to create a first zone for the bubbles to rise within the vessel, and to create a second zone for the accelerated liquid to break the bubbles into fine bubbles and for fluid to flow through the vessel. The fluid may include the accelerated portion of the liquid and fine bubbles. In still other examples, the superficial velocity of the gas phase in the vessel may be at least 30 mm/s. The sparger may be a sintered sparger or an orifice sparger. The thickness of the plate may be about 1 mm to about 25 mm. The accelerated liquid may have a velocity of about 8000 mm/s to about 17000 mm/s. In other examples, the accelerated liquid may have a velocity of about 12000 mm/s to about 17000 mm/s. In some examples, the bubbles injected into the liquid from the sparger may have a diameter of about 2 mm to about 20 mm. In another example, the bubbles injected into the liquid from the sparger may have a diameter of about 5 mm to about 15 mm, or from about 7 mm to about 13 mm. The fine bubbles may have a diameter of about 0.1 mm to about 5 mm, or about 0.2 mm to about 1.5 mm. The plurality of orifices may also be configured to accelerate at least 90% of the liquid in the vessel.

In another embodiment, the methods disclosed herein relate to generating fine bubbles that may include sparging gas into a vessel containing a liquid via at least one sparger positioned within the vessel and configured to inject bubbles into the liquid and accelerating a portion of the liquid in the vessel via a perforated plate positioned in an upper portion of the vessel, in which the liquid may be accelerated from the plate to break the bubbles into fine bubbles. In some examples, a superficial velocity of the gas phase in the vessel may be at least 30 mm/s. In other examples, the superficial velocity of the gas phase in the vessel may be from about 30 mm/s to about 80 mm/s. The sparger may be a sintered sparger or an orifice sparger. The liquid may be accelerated from the perforated plate at a velocity of about 8000 mm/s to about 17000 mm/s. In some examples, the liquid may be accelerated from the perforated plate at a velocity of about 12000 mm/s to about 17000 mm/s. The bubbles injected into the liquid from the sparger may have a diameter of about 2 mm to about 20 mm, or from greater than 5 mm to about 15 mm, or from about 7 mm to about 13 mm. Often the bubbles injected into the liquid from the sparger are not spherical. The injected bubbles may be referred to as coarse bubbles. In contrast, the fine bubbles may have a diameter of about 0.1 mm to about 5 mm, or about 0.2 mm to about 1.5 mm. The fine bubbles are typically spherical. The liquid stream may be introduced at a location proximate to the plate. The sparger may be positioned perpendicular or parallel to the plate, and a top or side surface of the sparger may be positioned from about 50 mm to about 300 mm, 500 mm, or 1000 mm from a bottom of the plate.

In yet another embodiment, the systems disclosed herein relate to a bioreactor that may include a vessel containing a liquid growth medium, a plate that may include a plurality of orifices positioned in an upper portion of the vessel and configured to accelerate at least a portion of the liquid growth medium in the vessel, a substrate that may include at least one C1 carbon source, at least one sparger positioned within the vessel with a surface of the sparger that may be positioned from about 50 mm to about 300 mm, 500 mm, or 1000 mm from a bottom of the plate and the sparger configured to inject substrate bubbles into the liquid growth medium. The sparger positioned within the vessel may create a first zone for the substrate bubbles to rise within the vessel, and a second zone for the accelerated liquid growth medium to break the substrate bubbles into substrate fine bubbles, and for fluid to flow through the vessel. The fluid may have the accelerated portion of the liquid growth medium and may have the substrate fine bubbles, and a culture of at least one microorganism in the liquid growth medium. The culture of at least one microorganism may anaerobically ferment the substrate to produce at least one fermentation product.

In still another embodiment, the methods disclosed herein relate to generating substrate fine bubbles in a bioreactor and may include sparging substrate bubbles of at least one C1 carbon source into a vessel containing a liquid growth medium via at least one sparger positioned within the vessel and accelerating a portion of the liquid growth medium in the vessel via a perforated plate positioned in an upper portion of the vessel. The liquid growth medium accelerated from the plate may break the substrate bubbles into substrate fine bubbles. A superficial velocity of the gas phase in the vessel may be at least 30 mm/s. A culture of at least one microorganism may be included in the liquid growth medium and may anaerobically ferment the substrate to produce at least one fermentation product.

The foregoing general description and following detailed description are examples and are intended to provide further explanation of the disclosure as claimed. Other objects, advantages, and novel features will be readily apparent to those skilled in the art from the following brief description of the drawings and detailed description of the disclosure.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

These and other aspects of the present disclosure, which should be considered in all its novel aspects, will become apparent from the following description, which is given by way of example only, with reference to the accompanying figures, in which:

FIG. 1 (FIG. 1): shows DNA sequencing diagram illustrating the successful deletion of the hoxKGZ locus in C. necator PHB-4.

FIG. 2 (FIG. 2): shows H₂:CO₂ uptake ratio plotted against dilution rate for C. necator ΔhoxKGZ.

FIG. 3 (FIG. 3): shows continuous ethylene production from CO₂ as the sole carbon source in a CSTR by a Cupriavidus necator strain with a disruption (deletion) to the membrane-bound hydrogenase and expressing an ethylene forming enzyme (pBBR1-Efe).

FIGS. 4A-4C (FIGS. 4A-4C): show open reading frames (ORFs) in the Δ35433 bp encompassing genes [E6A55_33395]-[E6A55_33560] of the C. necator genome.

### DETAILED DESCRIPTION

The following description of embodiments is given in general terms. The disclosure is further elucidated from the disclosure given under the heading “Examples” herein below, which provides experimental data supporting the disclosure, specific examples of various aspects of the disclosure, and means of performing the disclosure.

The inventors have surprisingly discovered a non-naturally occurring C1-fixing strain capable of continuously growing autotrophically at up to about 40° C.

### I. DEFINITIONS

Unless otherwise defined, the following terms as used throughout this specification are defined as follows:

The disclosure provides a non-naturally occurring strain and methods for the continuous production of nutritive ingredient, particularly by microbial fermentation of a gaseous substrate. A “microorganism” is a microscopic organism, especially a bacterium, archaeon, virus, or fungus. In an embodiment, the microorganism of the disclosure is a bacterium.

The term “non-naturally occurring” when used in reference to a microorganism is intended to mean that the microorganism has at least one genetic modification not found in a naturally occurring strain of the referenced species, including wild-type strains of the referenced species. Non-naturally occurring microorganisms are typically developed in a laboratory or research facility. The microorganisms of the disclosure are non-naturally occurring.

The term “feed” or “feed additive” or “feed composition,” as used herein, refer to any compound, preparation, mixture, or composition suitable for, or intended for, intake by an animal.

The term “nutritive” or “nutritive composition” or “nutritive ingredient,” as used herein, denotes usefulness as a nutritional component, such as in feed, feed compositions, feed additives, food, food compositions, or feed additives. The nutritive compositions can find use as a complete animal food or feed (diet) or as a supplement to animal food or feed.

The term “animal” includes humans.

The terms “genetic modification,” “genetic alteration,” or “genetic engineering” broadly refer to manipulation of the genome or nucleic acids of a microorganism by the hand of man. Likewise, the terms “genetically modified,” “genetically altered,” or “genetically engineered” refers to a microorganism containing such a genetic modification, genetic alteration, or genetic engineering. These terms may be used to differentiate a lab-generated microorganism from a naturally-occurring microorganism. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization. The microorganisms of the disclosure are genetically engineered.

“Recombinant” indicates that a nucleic acid, protein, or microorganism is the product of genetic modification, engineering, or recombination. Generally, the term “recombinant” refers to a nucleic acid, protein, or microorganism that contains or is encoded by genetic material derived from multiple sources, such as two or more different strains or species of microorganisms. The microorganisms of the disclosure are generally recombinant.

“Wild type” refers to the typical form of an organism, strain, gene, or characteristic as it occurs in nature, as distinguished from mutant or variant forms.

“Endogenous” refers to a nucleic acid or protein that is present or expressed in the wild-type or parental microorganism from which the microorganism of the disclosure is derived. For example, an endogenous gene is a gene that is natively present in the wild-type or parental microorganism from which the microorganism of the disclosure is derived. In one embodiment, the expression of an endogenous gene may be controlled by an exogenous regulatory element, such as an exogenous promoter.

“Exogenous” refers to a nucleic acid or protein that originates outside the microorganism of the disclosure. For example, an exogenous gene or enzyme may be artificially or recombinantly created and introduced to or expressed in the microorganism of the disclosure. An exogenous gene or enzyme may also be isolated from a heterologous microorganism and introduced to or expressed in the microorganism of the disclosure. Exogenous nucleic acids may be adapted to integrate into the genome of the microorganism of the disclosure or to remain in an extra-chromosomal state in the microorganism of the disclosure, for example, in a plasmid.

“Heterologous” refers to a nucleic acid or protein that is not present in the wild-type or parental microorganism from which the microorganism of the disclosure is derived. For example, a heterologous gene or enzyme may be derived from a different strain or species and introduced to or expressed in the microorganism of the disclosure. The heterologous gene or enzyme may be introduced to or expressed in the microorganism of the disclosure in the form in which it occurs in the different strain or species. Alternatively, the heterologous gene or enzyme may be modified in some way, e.g., by codon-optimizing it for expression in the microorganism of the disclosure or by engineering it to alter function, such as to reverse the direction of enzyme activity or to alter substrate specificity.

In particular, a heterologous nucleic acid or protein expressed in the microorganism described herein may be derived from Bacillus, Clostridium, Cupriavidus, Escherichia, Gluconobacter, Hyphomicrobium, Lysinibacillus, Paenibacillus, Pseudomonas, Sedimenticola, Sporosarcina, Streptomyces, Thermithiobacillus, Thermotoga, Zea, Klebsiella, Mycobacterium, Salmonella, Mycobacteroides, Staphylococcus, Burkholderia, Listeria, Acinetobacter, Shigella, Neisseria, Bordetella, Streptococcus, Enterobacter, Vibrio, Legionella, Xanthomonas, Serratia, Cronobacter, Cupriavidus, Helicobacter, Yersinia, Cutibacterium, Francisella, Pectobacterium, Arcobacter, Lactobacillus, Shewanella, Erwinia, Sulfurospirillum, Peptococcaceae, Thermococcus, Saccharomyces, Pyrococcus, Glycine, Homo, Ralstonia, Brevibacterium, Methylobacterium, Geobacillus, Bos, Gallus, Anaerococcus, Xenopus, Amblyrhynchus, Rattus, Mus, Sus, Rhodococcus, Rhizobium, Megasphaera, Mesorhizobium, Peptococcus, Agrobacterium, Campylobacter, Acetobacterium, Alkalibaculum, Blautia, Butyribacterium, Eubacterium, Moorella, Oxobacter, Sporomusa, Thermoanaerobacter, Schizosaccharomyces, Paenibacillus, Fictibacillus, Lysinibacillus, Ornithinibacillus, Halobacillus, Kurthia, Lentibacillus, Anoxybacillus, Solibacillus, Virgibacillus, Alicyclobacillus, Sporosarcina, Salimicrobium, Sporosarcina, Planococcus, Corynebacterium, Thermaerobacter, Sulfobacillus, or Symbiobacterium.

The terms “polynucleotide,” “nucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides or nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene products.”

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

The term “copolymer” is a composition comprising two or more species of monomers are linked in the same polymer chain of the disclosure.

“Enzyme activity,” or simply “activity,” refers broadly to enzymatic activity, including, but not limited, to the activity of an enzyme, the amount of an enzyme, or the availability of an enzyme to catalyze a reaction. Accordingly, “increasing” enzyme activity includes increasing the activity of an enzyme, increasing the amount of an enzyme, or increasing the availability of an enzyme to catalyze a reaction. Similarly, “decreasing” enzyme activity includes decreasing the activity of an enzyme, decreasing the amount of an enzyme, or decreasing the availability of an enzyme to catalyze a reaction.

“Mutated” refers to a nucleic acid or protein that has been modified in the microorganism of the disclosure compared to the wild-type or parental microorganism from which the microorganism of the disclosure is derived. In one embodiment, the mutation may be a deletion, insertion, or substitution in a gene encoding an enzyme. In another embodiment, the mutation may be a deletion, insertion, or substitution of one or more amino acids in an enzyme.

“Disrupted gene” refers to a gene that has been modified in some way to reduce or eliminate expression of the gene, regulatory activity of the gene, or activity of an encoded protein or enzyme. The disruption may partially inactivate, fully inactivate, or delete the gene or enzyme. The disruption may be a knockout (KO) mutation that fully eliminates the expression or activity of a gene, protein, or enzyme. The disruption may also be a knock-down that reduces, but does not entirely eliminate, the expression or activity of a gene, protein, or enzyme. The disruption may be anything that reduces, prevents, or blocks the biosynthesis of a product produced by an enzyme. The disruption may include, for example, a mutation in a gene encoding a protein or enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, RNAi, TALEN, siRNA, CRISPR, or CRISPRi) or protein which inhibits the expression of a protein or enzyme. The disruption may be introduced using any method known in the art. For the purposes of the present disclosure, disruptions are laboratory-generated, not naturally occurring.

A “parental microorganism” is a microorganism used to generate a microorganism of the disclosure. The parental microorganism may be a naturally-occurring microorganism (i.e., a wild-type microorganism) or a microorganism that has been previously modified (i.e., a mutant or recombinant microorganism). The microorganism of the disclosure may be modified to express or overexpress one or more enzymes that were not expressed or overexpressed in the parental microorganism. Similarly, the microorganism of the disclosure may be modified to contain one or more genes that were not contained by the parental microorganism. The microorganism of the disclosure may also be modified to not express or to express lower amounts of one or more enzymes that were expressed in the parental microorganism.

The microorganism of the disclosure may be derived from essentially any parental microorganism.

The terms “derived from” or “derivative” indicates that a nucleic acid, protein, or microorganism is modified or adapted from a different (e.g., a parental or wild-type) nucleic acid, protein, or microorganism, so as to produce a new nucleic acid, protein, or microorganism. Such modifications or adaptations typically include insertion, deletion, mutation, or substitution of nucleic acids or genes.

The microorganism of the disclosure may be further classified based on functional characteristics. For example, the microorganism of the disclosure may be or may be derived from a C1-fixing microorganism, an aerobe, an anaerobe, an acetogen, an ethanologen, a carboxydotroph, an autotroph, and/or a methanotroph. The microorganism of the disclosure may be selected from chemoautotroph, hydrogenotroph, knallgas, methanotroph, or any combination thereof. In some embodiments, the microorganism may be hydrogen-oxidizing, carbon monoxide-oxidizing, knallgas, or any combination thereof, with the capability to grow and synthesize biomass on gaseous carbon sources such as syngas and/or CO₂, such that the production microorganisms synthesize targeted chemical products under gas cultivation. The microorganisms and methods of the present disclosure can enable low cost synthesis of biochemicals, which can compete on price with petrochemicals and higher-plant derived amino acids, proteins, and other biological nutrients. In certain embodiments, these amino acids, proteins, and other biological nutrients may have a substantially lower price than amino acids, proteins, and other biological nutrients produced through heterotrophic or microbial phototrophic synthesis. Knallgas microbes, hydrogenotrophs, carboxydotrophs, and chemoautotrophs more broadly, are able to capture CO₂ or CO as their sole carbon source to support biological growth. In some embodiments, this growth includes the biosynthesis of amino acids and proteins. Knallgas microbes and other hydrogenotrophs can use H₂ as a source of reducing electrons for respiration and biochemical synthesis. In some embodiments of the present disclosure knallgas organisms and/or hydrogenotrophs and/or carboxydotrophs and/or other chemoautotrophic microorganisms are grown on a stream of gasses including but not limited to one or more of the following: CO₂; CO; H₂; along with inorganic minerals dissolved in aqueous solution. In some embodiments knallgas microbes and/or hydrogenotrophs and/or carboxydotrophs and/or other chemoautotrophic and/or methanotrophic microorganisms convert greenhouse gases into biomolecules including amino acids and proteins.

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism of the disclosure. For example, a C1-carbon source may comprise one or more of CO, CO₂, CH₄, CH₃OH, or CH₂O2. Preferably, the C1-carbon source comprises one or both of CO and CO₂. A “C1-fixing microorganism” is a microorganism that has the ability to produce one or more products from a C1-carbon source. Often, the microorganism of the disclosure is a C1-fixing bacterium. In a preferred embodiment, the microorganism of the disclosure is derived from a C1-fixing microorganism.

An “anaerobe” is a microorganism that does not require oxygen for growth. An anaerobe may react negatively or even die if oxygen is present above a certain threshold. However, some anaerobes are capable of tolerating low levels of oxygen (e.g., 0.000001-5% oxygen), sometimes referred to as “microoxic conditions.” Often, the microorganism of the disclosure is an anaerobe. In a preferred embodiment, the microorganism of the disclosure is derived from an anaerobe.

“Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3^{rd} edition, p. 354, New York, NY, 2006). All naturally occurring acetogens are C1-fixing, anaerobic, autotrophic, and non-methanotrophic. Often, the microorganism of the disclosure is an acetogen. In a preferred embodiment, the microorganism of the disclosure is derived from an acetogen.

An “ethanologen” is a microorganism that produces or is capable of producing ethanol. Often, the microorganism of the disclosure is an ethanologen. In a preferred embodiment, the microorganism of the disclosure is derived from an ethanologen.

An “autotroph” is a microorganism capable of growing in the absence of organic carbon. Instead, autotrophs use inorganic carbon sources, such as CO and/or CO₂. Often, the microorganism of the disclosure is an autotroph. In a preferred embodiment, the microorganism of the disclosure is derived from an autotroph.

A “carboxydotroph” is a microorganism capable of utilizing CO as a sole source of carbon and energy. Often, the microorganism of the disclosure is a carboxydotroph. In a preferred embodiment, the microorganism of the disclosure is derived from a carboxydotroph.

The term “heterotrophic” refers to organisms that cannot synthesize all the organic compounds needed by the organism to live and grow from carbon dioxide, and which must utilize organic compounds for growth.

The term “hydrogen-oxidizer” refers to microorganisms that utilize reduced H2 as an electron donor for the production of intracellular reducing equivalents and/or in respiration.

A “methanotroph” is a microorganism capable of utilizing methane as a sole source of carbon and energy. In certain embodiments, the microorganism of the disclosure is a methanotroph or is derived from a methanotroph. In other embodiments, the microorganism of the disclosure is not a methanotroph or is not derived from a methanotroph.

The term “knallgas” refers to the mixture of molecular hydrogen and oxygen gas. A “knallgas microorganism” is a microbe that can use hydrogen as an electron donor and oxygen as an electron acceptor in respiration for the generation of intracellular energy carriers such as Adenosine-5′-triphosphate (ATP).

The terms “oxyhydrogen” and “oxyhydrogen microorganism” can be used synonymously with “knallgas” and “knallgas microorganism” respectively. Knallgas microorganisms generally use molecular hydrogen by means of hydrogenases, with some of the electrons donated from H₂ being utilized for the reduction of NAD⁺ (and/or other intracellular reducing equivalents) and some of the electrons from H₂ being used for aerobic respiration. Knallgas microorganisms generally fix CO₂ autotrophically, through pathways including but not limited to the Calvin Cycle or the reverse citric acid cycle.

The term “methanogen” refers to a microorganism that generates methane as a product of anaerobic respiration.

The term “methylotroph” refers to microorganisms that can use reduced one-carbon compounds, such as but not limited to methanol or methane, as a carbon source and/or as an electron donor for their growth.

The term “green hydrogen” refers to hydrogen generated from clean electricity or surplus renewable energy sources.

The term “blue hydrogen” refers to hydrogen primarily generated from steam reforming of natural gas thereby producing hydrogen and carbon dioxide.

The term “grey hydrogen” refers to hydrogen primarily generated from steam reforming of natural gas thereby producing hydrogen and carbon dioxide, butthe carbon dioxide is not obtained through carbon capture and storage.

The term “pink hydrogen” refers to hydrogen generated through electrolysis from nuclear energy.

The term “turquoise hydrogen” refers to hydrogen generated by methane pyrolysis producing solid carbon and hydrogen.

The term “yellow hydrogen” refers to hydrogen generated by electrolysis using solar power.

The term “white hydrogen” refers to hydrogen that is naturally occurring hydrogen.

### II. MICROORGANISMS FOR THE PRODUCTION OF PRODUCTS THROUGH GAS FERMENTATION

As described above, however, the microorganism of the disclosure may also be derived from essentially any parental microorganism, such as a parental microorganism selected from the group consisting of Escherichia coli and Saccharomyces cerevisiae.

In another embodiment, the microorganism of the disclosure is an aerobic bacterium.

In one embodiment, the microorganism of the disclosure comprises aerobic hydrogen bacteria. In an embodiment, the aerobic bacteria comprising at least one disrupted gene.

A number of aerobic bacteria are known to be capable of carrying out fermentation for the disclosed methods and system. Examples of such bacteria that are suitable for use in the disclosure include bacteria of the genus Cupriavidus and Ralstonia. In some embodiments, the aerobic bacteria is Cupriavidus necator or Ralstonia eutropha. In some embodiments, the aerobic bacteria is Cupriavidus alkaliphilus. In some embodiments, the aerobic bacteria is Cupriavidus basilensis. In some embodiments, the aerobic bacteria is Cupriavidus campinensis. In some embodiments, the aerobic bacteria is Cupriavidus gilardii. In some embodiments, the aerobic bacteria is Cupriavidus laharis. In some embodiments, the aerobic bacteria is Cupriavidus metallidurans. In some embodiments, the aerobic bacteria is Cupriavidus nantongensis. In some embodiments, the aerobic bacteria is Cupriavidus numazuensis. In some embodiments, the aerobic bacteria is Cupriavidus oxalaticus. In some embodiments, the aerobic bacteria is Cupriavidus pampae. In some embodiments, the aerobic bacteria is Cupriavidus pauculus. In some embodiments, the aerobic bacteria is Cupriavidus pinatubonensis. In some embodiments, the aerobic bacteria is Cupriavidus plantarum. In some embodiments, the aerobic bacteria is Cupriavidus respiraculi. In some embodiments, the aerobic bacteria is Cupriavidus taiwanensis. In some embodiments, the aerobic bacteria is Cupriavidus yeoncheonensis.

In some embodiments, the strain is Cupriavidus necator DSM 428, DSM 531, or DSM541, or any derivatives thereof. In another embodiment, the strain is Cupriavidus necator DSM 34774. In some embodiments, the strain is SEQ ID NOs: 1-3. In some embodiments, the strain is a derivative of Cupriavidus necator DSM 34774. In another embodiment, the strain is a derivative of SEQ ID NOs: 1-3.

In some embodiments, the aerobic bacteria comprises one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or any combination thereof.

In the microorganisms of the disclosure, carbon flux is strategically diverted away from nonessential or undesirable products and towards products of interest. In certain embodiments, these disrupted genes divert carbon flux away from nonessential or undesirable metabolic nodes and through target metabolic nodes to improve production of products downstream of those target metabolic nodes. In an embodiment, limitation selected from nutrients, dissolved oxygen, or any combination thereof diverts carbon flux to desired products.

In an embodiment, the fermentation broth comprises the feed streams in combination with the aerobic microorganism in the bioreactor. In some embodiments, the feed streams, e.g., a carbon source feed stream, a flammable gas-containing stream, and an oxygen-containing gas feed stream, react with the microorganism in the bioreactor to at least partially form the fermentation broth (which may also include other products, byproducts, and other media fed to the bioreactor). The unreacted oxygen, or the oxygen that is not consumed by the microorganism, exists as both dissolved oxygen and gaseous oxygen in a dispersed gaseous phase within the fermentation broth. The same holds true for the other gases that are soluble. The dispersed gaseous phase, containing the unreacted components, e.g., oxygen, nitrogen, hydrogen, carbon dioxide and/or water vapor, rises to the headspace of the bioreactor.

In some embodiments, an oxygen-containing gas, e.g., air, can be fed directly into the fermentation broth. In one embodiment, the oxygen-containing gas can be an oxygen-enriched source, e.g., oxygen-enriched air or pure oxygen. In an embodiment, the oxygen-containing gas may comprise greater than 6.0 vol. % of oxygen, e.g., greater than 10.0 vol. %, greater than 20.0 vol. %, greater than 40.0 vol. %, greater than 60.0 vol. %, greater than 80.0 vol. %, or greater than 90.0 vol. %. In some embodiments, the oxygen-containing gas may be pure oxygen.

In one embodiment, the microorganism of the disclosure is capable of producing ethylene. One embodiment is directed to a recombinant C1-fixing microorganism capable of producing ethylene from a carbon source comprising a nucleic acid encoding a group of exogenous enzymes comprising at least one ethylene forming enzyme (EFE). In some embodiments the EFE is derived from Pseudomonas syringae. In an embodiment, the EFE has an E.C. number 1.13.12.19. The microorganism of an embodiment comprising at least one EFE having an E.C. number 1.13.12.19. The microorganism of an embodiment, further comprising a nucleic acid encoding a group of exogenous enzymes comprising at least one alpha-ketoglutarate permease (AKGP).

The microorganism of an embodiment, wherein a nucleic acid encoding a group of exogenous enzymes comprises at least one EFE, at least one AKGP, or any combination thereof. The microorganism of an embodiment, wherein a nucleic acid encoding a group of exogenous enzymes comprises at least one EFE and at least one AKGP. The microorganism of an embodiment, wherein the nucleotide encoding a group of exogenous enzymes is inserted into a bacterial vector plasmid, a high copy number bacterial vector plasmid, a bacterial vector plasmid having an inducible promoter, a nucleotide guide of a homologous recombination system, a CRISPR Cas system, or any combination thereof. In an embodiment, the promoter is a phosphate limited inducible promoter. In some embodiments, the promoter is a nitrogen limited promoter. In some embodiments, the promoter is an NtrC-P activated promoter. In some embodiments, the promoter is a H₂ inducible promoter. In one embodiment, the microorganism comprises an intracellular oxygen concentration limit. In another embodiment, the method limits intracellular oxygen concentration. In one embodiment, the method comprises a step of controlling dissolved oxygen. In an embodiment, the method comprises decreased ethylene production with decreased dissolved oxygen concentration. In some embodiments, the microorganism comprises a molecular switch. In some embodiments, the microorganism comprises an ability to switch the cellular burden under variable conditions.

In some embodiments, the microorganism is a natural or an engineered microorganism that is capable of converting a gaseous substrate as a carbon and/or energy source. In one embodiment, the gaseous substrate includes CO₂ as a carbon source. In some embodiments, the gaseous substrate includes H₂, and/or O₂ as an energy source. In one embodiment, the gaseous substrate includes a mixture of gases, comprising H₂ and/or CO₂ and/or CO.

In some embodiments, the gas fermentation product is selected from an alcohol, an acid, a diacid, an alkene, a terpene, an isoprene, and alkyne. In some embodiments, the method and microorganism disclosed herein are for the improved production of ethylene. In an embodiment, the method and microorganism disclosed herein are for the improved production of a gas fermentation product.

In one embodiment, the aerobic bacteria may produce a product such as acetone, isopropanol, 3-hydroxyisovaleryl-CoA, 3-hydroxyisovalerate, isobutylene, isopentenyl pyrophosphate, dimethylallyl pyrophosphate, isoprene, farnesene, 3-hydroxybutyryl-CoA, crotonyl-CoA, 3-hydroxybutyrate, 3-hydroxybutyrylaldehyde, 1,3-butanediol, 2-hydroxyisobutyryl-CoA, 2-hydroxyisobutyrate, butyryl-CoA, butyrate, butanol, caproate, hexanol, octanoate, octanol, 1,3-hexanediol, 2-buten-1-ol, isovaleryl-CoA, isovalerate, isoamyl alcohol, methacrolein, methyl-methacrylate, or any combination thereof.

In another embodiment, the bacteria of the disclosure may produce ethylene, ethanol, propane, acetate, 1-butanol, butyrate, 2,3-butanediol, lactate, butene, butadiene, methyl ethyl ketone (2-butanone), acetone, isopropanol, a lipid, 3-hydroxypropionate (3-HP), a terpene, isoprene, a fatty acid, 2-butanol, 1,2-propanediol, lpropanol, lhexanol, loctanol, chorismate-derived products, 3hydroxybutyrate, 1,3butanediol, 2-hydroxyisobutyrate or 2-hydroxyisobutyric acid, isobutylene, adipic acid, keto-adipic acid, 1,3hexanediol, 3-methyl-2-butanol, 2-buten-1-ol, isovalerate, isoamyl alcohol, and monoethylene glycol, or any combination thereof.

The disclosure provides microorganisms capable of producing ethylene comprising culturing the microorganism of the disclosure in the presence of a substrate, whereby the microorganism produces ethylene.

As used herein, the terms “intermediate” and “precursor” can be used interchangeably to refer to a substance, such as a molecule, compound, or protein, that is produced upstream of a particular product. The intermediate may be directly upstream of the product. The intermediate may be indirectly upstream of the product. For example, in the exemplary reaction “compound A” 4 “compound B” 4 “compound C” 4 “compound D”, “compound” C is an intermediate that is directly upstream of the product, “compound D,” and “compound B” is an intermediate that is indirectly upstream of the product, “compound D.”

The enzymes of the disclosure may be codon optimized for expression in the microorganism of the disclosure. “Codon optimization” refers to the mutation of a nucleic acid, such as a gene, for optimized or improved translation of the nucleic acid in a particular strain or species. Codon optimization may result in faster translation rates or higher translation accuracy. In a preferred embodiment, the genes of the disclosure are codon optimized for expression in the microorganism of the disclosure. Although codon optimization refers to the underlying genetic sequence, codon optimization often results in improved translation and, thus, improved enzyme expression. Accordingly, the enzymes of the disclosure may also be described as being codon optimized.

One or more of the enzymes of the disclosure may be overexpressed. “Overexpressed” refers to an increase in expression of a nucleic acid or protein in the microorganism of the disclosure compared to the wild-type or parental microorganism from which the microorganism of the disclosure is derived. Overexpression may be achieved by any means known in the art, including modifying gene copy number, gene transcription rate, gene translation rate, or enzyme degradation rate.

The enzymes of the disclosure may comprise a disruptive mutation. A “disruptive mutation” refers to a mutation that reduces or eliminates (i.e., “disrupts”) the expression or activity of a gene or enzyme. The disruptive mutation may partially inactivate, fully inactivate, or delete the gene or enzyme. The disruptive mutation may be a knockout (KO) mutation. The disruptive mutation may be any mutation that reduces, prevents, or blocks the biosynthesis of a product produced by an enzyme. The disruptive mutation may include, for example, a mutation in a gene encoding an enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, siRNA, CRISPR) or protein which inhibits the expression of an enzyme. The disruptive mutation may be introduced using any method known in the art.

Introduction of a disruptive mutation results in a microorganism of the disclosure that produces no target product or substantially no target product or a reduced amount of target product compared to the parental microorganism from which the microorganism of the disclosure is derived. For example, the microorganism of the disclosure may produce no target product or at least about 1%, 3%, 5%, 10%, 20%, 30%, 40%, 50%, 60%, 70%, 80%, 90%, or 95% less target product than the parental microorganism. For example, the microorganism of the disclosure may produce less than about 0.001, 0.01, 0.10, 0.30, 0.50, or 1.0 g/L target product.

Although exemplary sequences and sources for enzymes are provided herein, the disclosure is by no means limited to these sequences and sources—it also encompasses variants. The term “variants” includes nucleic acids and proteins whose sequence varies from the sequence of a reference nucleic acid and protein, such as a sequence of a reference nucleic acid and protein disclosed in the prior art or exemplified herein. The disclosure may be practiced using variant nucleic acids or proteins that perform substantially the same function as the reference nucleic acid or protein. For example, a variant protein may perform substantially the same function or catalyze substantially the same reaction as a reference protein. A variant gene may encode the same or substantially the same protein as a reference gene. A variant promoter may have substantially the same ability to promote the expression of one or more genes as a reference promoter.

Such nucleic acids or proteins may be referred to herein as “functionally equivalent variants.” By way of example, functionally equivalent variants of a nucleic acid may include allelic variants, fragments of a gene, mutated genes, polymorphisms, and the like. Homologous genes from other microorganisms are also examples of functionally equivalent variants. Functionally equivalent variants also include nucleic acids whose sequence varies as a result of codon optimization for a particular microorganism. A functionally equivalent variant of a nucleic acid will preferably have at least approximately 70%, approximately 80%, approximately 85%, approximately 90%, approximately 95%, approximately 98%, or greater nucleic acid sequence identity (percent homology) with the referenced nucleic acid. A functionally equivalent variant of a protein will preferably have at least approximately 70%, approximately 80%, approximately 85%, approximately 90%, approximately 95%, approximately 98%, or greater amino acid identity (percent homology) with the referenced protein. The functional equivalence of a variant nucleic acid or protein may be evaluated using any method known in the art.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

Nucleic acids may be delivered to a microorganism of the disclosure using any method known in the art. For example, nucleic acids may be delivered as naked nucleic acids or may be formulated with one or more agents, such as liposomes. The nucleic acids may be DNA, RNA, cDNA, or combinations thereof, as is appropriate. Restriction inhibitors may be used in certain embodiments. Additional vectors may include plasmids, viruses, bacteriophages, cosmids, and artificial chromosomes. In a preferred embodiment, nucleic acids are delivered to the microorganism of the disclosure using a plasmid. By way of example, transformation (including transduction or transfection) may be achieved by electroporation, ultrasonication, polyethylene glycol-mediated transformation, chemical or natural competence, protoplast transformation, prophage induction, or conjugation. In certain embodiments having active restriction enzyme systems, it may be necessary to methylate a nucleic acid before introduction of the nucleic acid into a microorganism.

Furthermore, nucleic acids may be designed to comprise a regulatory element, such as a promoter, to increase or otherwise control expression of a particular nucleic acid. The promoter may be a constitutive promoter or an inducible promoter. The promoter may be a Wood-Ljungdahl pathway promoter, a ferredoxin promoter, a pyruvate ferredoxin oxidoreductase promoter, an Rnf complex operon promoter, an ATP synthase operon promoter, or a phosphotransacetylase/acetate kinase operon promoter.

It should be appreciated that the disclosure may be practiced using nucleic acids whose sequence varies from the sequences specifically exemplified herein provided they perform substantially the same function. For nucleic acid sequences that encode a protein or peptide this means that the encoded protein or peptide has substantially the same function. For nucleic acid sequences that represent promoter sequences, the variant sequence will have the ability to promote expression of one or more genes. Such nucleic acids may be referred to herein as “functionally equivalent variants.” By way of example, functionally equivalent variants of a nucleic acid include allelic variants, fragments of a gene, genes which include mutations (deletion, insertion, nucleotide substitutions and the like) and/or polymorphisms and the like. Homologous genes from other microorganisms may also be considered as examples of functionally equivalent variants of the sequences specifically exemplified herein.

The phrase “functionally equivalent variants” should also be taken to include nucleic acids whose sequence varies as a result of codon optimisation for a particular organism. “Functionally equivalent variants” of a nucleic acid herein will preferably have at least approximately 70%, preferably approximately 80%, more preferably approximately 85%, preferably approximately 90%, preferably approximately 95% or greater nucleic acid sequence identity with the nucleic acid identified.

It should also be appreciated that the disclosure may be practiced using polypeptides whose sequence varies from the amino acid sequences specifically exemplified herein. These variants may be referred to herein as “functionally equivalent variants.” A functionally equivalent variant of a protein or a peptide includes those proteins or peptides that share at least 40%, preferably 50%, preferably 60%, preferably 70%, preferably 75%, preferably 80%, preferably 85%, preferably 90%, preferably 95% or greater amino acid identity with the protein or peptide identified and has substantially the same function as the peptide or protein of interest. Such variants include within their scope fragments of a protein or peptide wherein the fragment comprises a truncated form of the polypeptide wherein deletions may be from 1 to 5, to 10, to 15, to 20, to 25 amino acids, and may extend from residue 1 through 25 at either terminus of the polypeptide, and wherein deletions may be of any length within the region; or may be at an internal location. Functionally equivalent variants of the specific polypeptides herein should also be taken to include polypeptides expressed by homologous genes in other species of bacteria, for example as exemplified in the previous paragraph.

The microorganisms of the disclosure may be prepared from a parental microorganism and one or more exogenous nucleic acids using any number of techniques known in the art for producing recombinant microorganisms. By way of example only, transformation (including transduction or transfection) may be achieved by electroporation, ultrasonication, polyethylene glycol-mediated transformation, chemical or natural competence, or conjugation. Suitable transformation techniques are described for example in, Sambrook J, Fritsch E F, Maniatis T: Molecular Cloning: A laboratory Manual, Cold Spring Harbour Laboratory Press, Cold Spring Harbour, 1989.

In certain embodiments, due to the restriction systems which are active in the microorganism to be transformed, it is necessary to methylate the nucleic acid to be introduced into the microorganism. This can be done using a variety of techniques, including those described below, and further exemplified in the Examples section herein after.

By way of example, in one embodiment, a recombinant microorganism of the disclosure is produced by a method comprises the following steps: introduction into a shuttle microorganism of (i) of an expression construct/vector as described herein and (ii) a methylation construct/vector comprising a methyltransferase gene; expression of the methyltransferase gene; isolation of one or more constructs/vectors from the shuttle microorganism; and, introduction of the one or more construct/vector into a destination microorganism.

In one embodiment, the methyltransferase gene of step B is expressed constitutively. In another embodiment, expression of the methyltransferase gene of step B is induced.

The shuttle microorganism is a microorganism, preferably a restriction negative microorganism that facilitates the methylation of the nucleic acid sequences that make up the expression construct/vector. In a particular embodiment, the shuttle microorganism is a restriction negative E. coli, Bacillus subtilis, or Lactococcus lactis.

The methylation construct/vector comprises a nucleic acid sequence encoding a methyltransferase.

Once the expression construct/vector and the methylation construct/vector are introduced into the shuttle microorganism, the methyltransferase gene present on the methylation construct/vector is induced. Induction may be by any suitable promoter system although in one particular embodiment of the disclosure, the methylation construct/vector comprises an inducible lac promoter and is induced by addition of lactose or an analogue thereof, more preferably isopropyl-β-D-thiogalactoside (IPTG). Other suitable promoters include the ara, tet, or T7 system. In a further embodiment of the disclosure, the methylation construct/vector promoter is a constitutive promoter.

In a particular embodiment, the methylation construct/vector has an origin of replication specific to the identity of the shuttle microorganism so that any genes present on the methylation construct/vector are expressed in the shuttle microorganism. Preferably, the expression construct/vector has an origin of replication specific to the identity of the destination microorganism so that any genes present on the expression construct/vector are expressed in the destination microorganism.

Expression of the methyltransferase enzyme results in methylation of the genes present on the expression construct/vector. The expression construct/vector may then be isolated from the shuttle microorganism according to any one of a number of known methods. By way of example only, the methodology described in the Examples section described hereinafter may be used to isolate the expression construct/vector.

In one particular embodiment, both construct/vector are concurrently isolated.

The expression construct/vector may be introduced into the destination microorganism using any number of known methods. However, by way of example, the methodology described in the Examples section hereinafter may be used. Since the expression construct/vector is methylated, the nucleic acid sequences present on the expression construct/vector are able to be incorporated into the destination microorganism and successfully expressed.

It is envisaged that a methyltransferase gene may be introduced into a shuttle microorganism and over-expressed. Thus, in one embodiment, the resulting methyltransferase enzyme may be collected using known methods and used in vitro to methylate an expression plasmid. The expression construct/vector may then be introduced into the destination microorganism for expression. In another embodiment, the methyltransferase gene is introduced into the genome of the shuttle microorganism followed by introduction of the expression construct/vector into the shuttle microorganism, isolation of one or more constructs/vectors from the shuttle microorganism and then introduction of the expression construct/vector into the destination microorganism.

It is envisaged that the expression construct/vector and the methylation construct/vector as defined above may be combined to provide a composition of matter. Such a composition has particular utility in circumventing restriction barrier mechanisms to produce the recombinant microorganisms of the disclosure.

In one particular embodiment, the expression construct/vector and/or the methylation construct/vector are plasmids.

Persons of ordinary skill in the art will appreciate a number of suitable methyltransferases of use in producing the microorganisms of the disclosure. However, by way of example the Bacillus subtilis phage (ΦT1 methyltransferase and the methyltransferase described in the Examples herein after may be used. Nucleic acids encoding suitable methyltransferases will be readily appreciated having regard to the sequence of the desired methyltransferase and the genetic code.

Any number of constructs/vectors adapted to allow expression of a methyltransferase gene may be used to generate the methylation construct/vector.

In one embodiment, the substrate comprises CO₂ and an energy source. In some embodiments, the substrate comprises CO₂ and an energy source. In an embodiment, the substrate comprises CO₂, H₂, and O₂. In some embodiments, the substrate comprises CO₂ and any suitable energy source. In one embodiment, the substrate comprises CO. In one embodiment, the substrate comprises CO2 and CO. In another embodiment, the substrate comprises CO2 and H2. In another embodiment, the substrate comprises CO2 and CO and H2.

“Substrate” refers to a carbon and/or energy source for the microorganism of the disclosure. Often, the substrate is gaseous and comprises a C1-carbon source, for example, CO, CO₂, and/or CH₄. Preferably, the substrate comprises a C1-carbon source of CO or CO+CO₂. The substrate may further comprise other non-carbon components, such as H₂, N₂, or electrons. In other embodiments, however, the substrate may be a carbohydrate, such as sugar, starch, fiber, lignin, cellulose, or hemicellulose or a combination thereof. For example, the carbohydrate may be fructose, galactose, glucose, lactose, maltose, sucrose, xylose, or some combination thereof. In some embodiments, the substrate does not comprise (D)-xylose (Alkim, Microb Cell Fact, 14: 127, 2015). In some embodiments, the substrate does not comprise a pentose such as xylose (Pereira, Metab Eng, 34: 80-87, 2016). In some embodiments, the substrate may comprise both gaseous and carbohydrate substrates (mixotrophic fermentation). The substrate may further comprise other non-carbon components, such as H₂, N₂, or electrons.

In some embodiments, the gaseous substrate generally comprises at least some amount of CO, such as about 1, 2, 5, 10, 20, 30, 40, 50, 60, 70, 80, 90, or 100 mol % CO. The gaseous substrate may comprise a range of CO, such as about 20-80, 30-70, or 40-60 mol % CO. Preferably, the gaseous substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the gaseous substrate may comprise a relatively low amount of CO, such as about 1-10 or 1-20 mol % CO. The microorganism of the disclosure typically converts at least a portion of the CO in the gaseous substrate to a product. In some embodiments, the gaseous substrate comprises no or substantially no (<1 mol %) CO.

The gaseous substrate may comprise some amount of H₂. For example, the gaseous substrate may comprise about 1, 2, 5, 10, 15, 20, or 30 mol % H₂. In some embodiments, the gaseous substrate may comprise a relatively high amount of H₂, such as about 60, 70, 80, or 90 mol % H₂. In further embodiments, the gaseous substrate comprises no or substantially no (<1 mol %) H₂.

The gaseous substrate may comprise some amount of CO₂. For example, the gaseous substrate may comprise about 1-80 or 1-30 mol % CO₂. In some embodiments, the gaseous substrate may comprise less than about 20, 15, 10, or 5 mol % CO₂. In another embodiment, the gaseous substrate comprises no or substantially no (<1 mol %) CO₂.

The gaseous substrate may also be provided in alternative forms. For example, the gaseous substrate may be dissolved in a liquid or adsorbed onto a solid support.

The gaseous substrate and/or C1-carbon source may be a waste gas or an off gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the gaseous substrate and/or C1-carbon source may be captured from the industrial process before it is emitted into the atmosphere, using any convenient method.

The gaseous substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The terms “feedstock” when used in the context of the stream flowing into a gas fermentation bioreactor (i.e., gas fermenter) or “gas fermentation feedstock” should be understood to encompass any material (solid, liquid, or gas) or stream that can provide a substrate and/or C1-carbon source to a gas fermenter or bioreactor either directly or after processing of the feedstock.

The term “waste gas” or “waste gas stream” may be used to refer to any gas stream that is either emitted directly, flared with no additional value capture, or combusted for energy recovery purposes.

The terms “synthesis gas” or “syngas” refers to a gaseous mixture that contains at least one carbon source, such as carbon monoxide (CO), carbon dioxide (CO₂), or any combination thereof, and, optionally, hydrogen (H₂) that can used as a feedstock for the disclosed gas fermentation processes and can be produced from a wide range of carbonaceous material, both solid and liquid.

The substrate and/or C1-carbon source may be a waste gas obtained as a byproduct of an industrial process or from another source, such as automobile exhaust fumes, biogas, landfill gas, direct air capture, or from electrolysis. The substrate and/or C1-carbon source may be syngas generated by pyrolysis, torrefaction, or gasification. In other words, carbon in waste material may be recycled by pyrolysis, torrefaction, or gasification to generate syngas which is used as the substrate and/or C1-carbon source. The substrate and/or C1-carbon source may be a gas comprising methane.

In certain embodiments, the industrial process is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, geological reservoirs, gas from fossil resources such as natural gas coal and oil, or any combination thereof.

Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. In these embodiments, the substrate and/or C1-carbon source may be captured from the industrial process before it is emitted into the atmosphere, using any known method.

The substrate and/or C1-carbon source may be synthesis gas known as syngas, which may be obtained from reforming, partial oxidation, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of biogas. Examples of reforming processes include, steam methane reforming, steam naphtha reforming, reforming of natural gas, reforming of biogas, reforming of landfill gas, naphtha reforming, and dry methane reforming. Examples of partial oxidation processes include thermal and catalytic partial oxidation processes, catalytic partial oxidation of natural gas, partial oxidation of hydrocarbons. Examples of municipal solid waste include tires, plastics, fibers, such as in shoes, apparel, and textiles. Municipal solid waste may be simply landfill-type waste. The municipal solid waste may be sorted or unsorted. Examples of biomass may include lignocellulosic material and may also include microbial biomass. Lignocellulosic material may include agriculture waste and forest waste.

The substrate and/or C1-carbon source may be a gas stream comprising methane. Such a methane containing gas may be obtained from fossil methane emission such as during fracking, wastewater treatment, livestock, agriculture, and municipal solid waste landfills. It is also envisioned that the methane may be burned to produce electricity or heat, and the C1 byproducts may be used as the substrate or carbon source.

The composition of the gaseous substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

Regardless of the source or precise content of the gas used as a feedstock, the feedstock may be metered (e.g., for carbon credit calculations or mass balancing of sustainable carbon with overall products) into a bioreactor in order to maintain control of the follow rate and amount of carbon provided to the culture. Similarly, the output of the bioreactor may be metered (e.g., for carbon credit calculations or mass balancing of sustainable carbon with overall products) or comprise a valved connection that can control the flow of the output and products (e.g., ethylene, ethanol, acetate, 1-butanol, etc.) produced via fermentation. Such a valve or metering mechanism can be useful for a variety of purposes including, but not limited to, slugging of product through a connected pipeline and measuring the amount of output from a given bioreactor such that if the product is mixed with other gases or liquids the resulting mixture can later be mass balanced to determine the percentage of the product that was produced from the bioreactor.

In certain embodiments, the fermentation is performed in the absence of carbohydrate substrates, such as sugar, starch, fiber, lignin, cellulose, or hemicellulose.

The term “electrolysis process”, may include any substrate leaving the electrolysis process. In various instances, the electrolysis process is comprised of CO, H₂, or combinations thereof. In certain instances, the electrolysis process may contain portions of unconverted CO₂. Preferably, the electrolysis process is fed from the electrolysis process to the fermentation process.

The terms “improving the economics”, “optimizing the economics” and the like, when used in relationship to a fermentation process, include, but are not limited to, the increase of the amount of one or more of the products produced by the fermentation process during periods of time in which the value of the products produced is high relative to the cost of producing such products. The economics of the fermentation process may be improved by way of increasing the supply of feedstock to the bioreactor, which may be achieved for instance by supplementing the C1 feedstock from the industrial process with electrolysis process from the electrolysis process. The additional supply of feedstock may result in the increased efficiency of the fermentation process. Another means of improving the economics of the fermentation process is to select feedstock based upon the relative cost of the feedstock available. For example, when the cost of the C1 feedstock from the industrial process is higher than the cost of the electrolysis process from the electrolysis process, the electrolysis process may be utilized to displace at least a portion of the C1 feedstock. By selecting feedstock based upon the cost of such feedstock the cost of producing the resulting fermentation product is reduced.

The electrolysis process is capable of supplying feedstock comprising one or both of H₂ and CO. The “cost per unit of electrolysis process” may be expressed in terms of any given product produced by the fermentation process and any electrolysis process, for example for the production of ethanol with the electrolysis process defined as H₂, the cost per unit of electrolysis process is defined by the following equation:

$$( \frac{\$ z}{MWh} ) \times ( \frac{1 MWh}{3.6 {GJ}_{electricity}} ) \times ( x \frac{{GJ}_{electricity}}{{GJ}_{H 2}} ) \times ( y \frac{{GJ}_{H 2}}{{GJ}_{ethanol}} )$$

where z represents the cost of power, x represents the electrolysis process efficiency, and y represents the yield of ethanol.

For the production of ethanol with electrolysis process defined as CO, the cost per unit of electrolysis process is defined by the following equation:

$$( \frac{\$ z}{MWh} ) \times ( \frac{1 MWh}{3.6 {GJ}_{electricity}} ) \times ( x \frac{{GJ}_{electricity}}{{GJ}_{CO}} ) \times ( y \frac{{GJ}_{CO}}{{GJ}_{ethanol}} )$$

where z represents the cost of power, x represents the electrolysis process efficiency, and y represents the yield of ethanol.

In addition to the cost of feedstock, the fermentation process includes “production costs.” The “production costs” exclude the cost of the feedstock. “Production costs”, “marginal cost of production”, and the like, include the variable operating costs associated with running the fermentation process. This value may be dependent on the product being produced. The marginal cost of production may be represented by a fixed cost per unit of product, which may be represented in terms of the heating value of combustion of the product. For example, the calculation of the marginal cost of production for ethanol is defined by the following equation:

$$( \frac{\$ c}{metric ton} ) \times ( \frac{1 metric ton}{26.8 {GJ}_{ethanol}} )$$

where c represents the variable operating costs associated with running the bioreactor and 26.8 GJ represents the lower heating value of combustion of ethanol. In certain instances, the variable operating costs associated with running the bioreactor, c, is $200 for ethanol excluding the price of H₂/CO/CO₂.

The fermentation process is capable of producing a number of products. Each product defining a different value. The “value of the product” may be determined based upon the current market price of the product and the heating value of combustion of the product. For example, the calculation for the value of ethanol is defined by the following equation:

$$( \frac{\$ z}{metric ton} ) \times ( \frac{1 metric ton}{26.8 {GJ}_{ethanol}} )$$

where z is the current value of ethanol per metric ton and 26.8 GJ represents the lower heating value of combustion of ethanol.

To optimize the economics of the fermentation process, the value of the product produced must exceed the “cost of producing” such product. The cost of producing a product is defined as the sum of the “cost of feedstock” and the “marginal cost of production.” The economics of the fermentation process may be expressed in terms of a ratio defined by the value of product produced compared to the cost of producing such product. The economics of the fermentation process is improved as the ratio of the value of the product compared to the cost of producing such product increases. The economics of the fermentation process may be dependent on the value of the product produced, which may change dependent, at least in part, on the fermentation process implemented, including but not limited to the bacterial culture and/or the composition of the gas used in the fermentation process. When ethanol is the product produced by the fermentation process the economics may be determined by the following ratio:

$$( \frac{\$ z}{{GJ}_{ethanol}} ) : ( \frac{\$ x}{{GJ}_{ethanol}} ) + ( \frac{\$ y}{{GJ}_{ethanol}} )$$

where z represents the value of ethanol, x represents the cost of feedstock, and y represents the marginal cost of production (excluding feedstock).

The terms “increasing the efficiency”, “increased efficiency” and the like, when used in relation to a fermentation process, include, but are not limited to, increasing one or more of the rate of growth of microorganisms catalysing the fermentation, the growth and/or product production rate at elevated product concentrations, the volume of desired product produced per volume of substrate consumed, the rate of production or level of production of the desired product, and the relative proportion of the desired product produced compared with other by-products of the fermentation. In certain instances, the electrolysis process increases the efficiency of the fermentation process.

The terms “electrolysis module” and “electrolyzer” can be used interchangeably to refer to a unit that uses electricity to drive a non-spontaneous reaction. Electrolysis technologies are known in the art. Exemplary processes include alkaline water electrolysis, proton or anion exchange membrane (PEM, AEM) electrolysis, and solid oxide electrolysis (SOE) (Ursua et al., Proceedings of the IEEE 100(2):410-426, 2012; Jhong et al., Current Opinion in Chemical Engineering 2:191-199, 2013). The term “faradaic efficiency” is a value that references the number of electrons flowing through an electrolyzer and being transferred to a reduced product rather than to an unrelated process. SOE modules operate at elevated temperatures. Below the thermoneutral voltage of an electrolysis module, an electrolysis reaction is endothermic. Above the thermoneutral voltage of an electrolysis module, an electrolysis reaction is exothermic. In some embodiments, an electrolysis module is operated without added pressure. In some embodiments, an electrolysis module is operated at a pressure of 5-10 bar.

A “CO₂ electrolysis module” refers to a unit capable of splitting CO₂ into CO and O₂ and is defined by the following stoichiometric reaction: 2CO₂+electricity→2CO+O₂. The use of different catalysts for CO₂ reduction impact the end product. Catalysts including, but not limited to, Au, Ag, Zn, Pd, and Ga catalysts, have been shown effective for the production of CO from CO₂. In some embodiments, the pressure of a gas stream leaving a CO₂ electrolysis module is approximately 5-7 bar.

“H₂ electrolysis module,” “water electrolysis module,” and “H₂O electrolysis module” refer to a unit capable of splitting H₂O, in the form of steam, into H₂ and O₂ and is defined by the following stoichiometric reaction: 2H₂O+electricity→2H₂+O₂. An H₂O electrolysis module reduces protons to H₂ and oxidizes O²⁻ to O₂. H₂ produced by electrolysis can be blended with a C1-comprising gaseous substrate as a means to supply additional feedstock and to improve substrate composition.

H₂ and CO₂ electrolysis modules have 2 gas outlets. One side of the electrolysis module, the anode, comprises H₂ or CO (and other gases such as unreacted water vapor or unreacted CO₂). The second side, the cathode, comprises O₂ (and potentially other gases). The composition of a feedstock being passed to an electrolysis process may determine the presence of various components in a CO stream. For instance, the presence of inert components, such as CH₄ and/or N₂, in a feedstock may result in one or more of those components being present in the CO-enriched stream. Additionally, in some electrolyzers, O₂ produced at the cathode crosses over to the anode side where CO is generated and/or CO crosses over to the anode side, leading to cross contamination of the desired gas products.

The term “separation module” is used to refer to a technology capable of dividing a substance into two or more components. For example, an “O₂ separation module” may be used to separate an O₂-comprising gaseous substrate into a stream comprising primarily O₂ (also referred to as an “O₂-enriched stream” or “O₂-rich gas”) and a stream that does not primarily comprise O₂, comprises no O₂, or comprises only trace amounts of O2 (also referred to as an “02-lean stream” or “02-depleted stream”).

As used herein, the terms “enriched stream,” “rich gas,” “high purity gas,” and the like refer to a gas stream having a greater proportion of a particular component following passage through a module, such as an electrolysis module, as compared to the proportion of the component in the input stream into the module. For example, a “CO-enriched stream” may be produced upon passage of a CO₂-comprising gaseous substrate through a CO₂ electrolysis module. An “H₂-enriched stream” may be produced upon passage of a water gaseous substrate through an H₂ electrolysis module. An “O₂-enriched stream” emerges automatically from the anode of a CO₂ or H₂ electrolysis module; an “O₂-enriched stream” may also be produced upon passage of an O₂-comprising gaseous substrate through an O₂ separation module. A “CO₂-enriched stream” may be produced upon passage of a CO₂-comprising gaseous substrate through a CO₂ concentration module.

As used herein, the terms “lean stream,” “depleted gas,” and the like refer to a gas stream having a lesser proportion of a particular component following passage through a module, such as a concentration module or a separation module, as compared to the proportion of the component in the input stream into the module. For example, an O₂-lean stream may be produced upon passage of an O₂-comprising gaseous substrate through an O₂ separation module. The O₂-lean stream may comprise unreacted CO₂ from a CO₂ electrolysis module. The O₂-lean stream may comprise trace amounts of O₂ or no O₂. A “CO₂-lean stream” may be produced upon passage of a CO₂-comprising gaseous substrate through a CO₂ concentration module. The CO₂-lean stream may comprise CO, H₂, and/or a constituent such as a microbe inhibitor or a catalyst inhibitor. The CO₂-lean stream may comprise trace amounts of CO₂ or no CO₂.

In embodiments, the disclosure provides an integrated process wherein the pressure of the gas stream is capable of being increased and/or decreased. The term “pressure module” refers to a technology capable of producing (i.e., increasing) or decreasing the pressure of a gas stream. The pressure of the gas may be increased and/or decreased through any suitable means, for example one or more compressor and/or valve. In certain instances, a gas stream may have a lower than optimum pressure, or the pressure of the gas stream may be higher than optimal, and thus, a valve may be included to reduce the pressure. A pressure module may be located before or after any module described herein. For example, a pressure module may be utilized prior to a removal module, prior to a concentration module, prior to an electrolysis module, and/or prior to a CO-consuming process.

A “pressurized gas stream” refers to a gaseous substrate that has passed through a pressure module. A “pressurized gas stream” may also be used to refer to a gas stream that meets the operating pressure requirements of a particular module.

The terms “post-CO-consuming process gaseous substrate,” “post-CO-consuming process tail gas,” “tail gas,” and the like may be used interchangeably to refer to a gas that has passed through a CO-consuming process. The post-CO-consuming process gaseous substrate may comprise unreacted CO, unreacted H₂, and/or CO₂ produced (or not taken up in parallel) by the CO-consuming process. The post-CO-consuming process gaseous substrate may further be passed to one or more of a pressure module, a removal module, a CO₂ concentration module, and/or an electrolysis module. In some embodiments, a “post-CO-consuming process gaseous substrate” is a post-fermentation gaseous substrate.

The term “desired composition” is used to refer to the desired level and types of components in a substance, such as, for example, of a gas stream. More particularly, a gas is considered to have a “desired composition” if it contains a particular component (i.e., CO, H₂, and/or CO₂) and/or contains a particular component at a particular proportion and/or does not comprise a particular component (i.e., a contaminant harmful to the microorganisms) and/or does not comprise a particular component at a particular proportion. More than one component may be considered when determining whether a gas stream has a desired composition.

In addition to ethylene, the microorganisms of the disclosure may be cultured with the gaseous substrate to produce one or more products. For instance, the microorganism may produce or may be engineered to produce ethanol (WO 2007/117157, U.S. Pat. No. 7,972,824), acetate (WO 2007/117157, U.S. Pat. No. 7,972,824), 1-butanol (WO 2008/115080, U.S. Pat. No. 8,293,509, WO 2012/053905, U.S. Pat. No. 9,359,611 and WO 2017/066498, U.S. Pat. No. 9,738,875), butyrate (WO 2008/115080, U.S. Pat. No. 8,293,509), 2,3-butanediol (WO 2009/151342, U.S. Pat. No. 8,658,408 and WO 2016/094334, U.S. Pat. No. 10,590,406), lactate (WO 2011/112103, U.S. Pat. No. 8,900,836), butene (WO 2012/024522, US2012/045807), butadiene (WO 2012/024522, US 2012/045807), methyl ethyl ketone (2-butanone) (WO 2012/024522, US 2012/045807 and WO 2013/185123, U.S. Pat. No. 9,890,384), ethanol which is then converted to ethylene (WO 2012/026833, US 2013/157,322), acetone (WO 2012/115527, U.S. Pat. No. 9,410,130), isopropanol (WO 2012/115527 U.S. Pat. No. 9,410,130), lipids (WO 2013/036147 U.S. Pat. No. 9,068,202), 3-hydroxypropionate (3-HP) (WO 2013/180581, U.S. Pat. No. 9,994,878), terpenes, including isoprene (WO 2013/180584, U.S. Pat. No. 10,913,958), fatty acids (WO 2013/191567 U.S. Pat. No. 9,347,076), 2-butanol (WO 2013/185123 U.S. Pat. No. 9,890,384), 1,2-propanediol (WO 2014/036152, U.S. Pat. No. 9,284,564), 1-propanol (WO 2014/0369152, U.S. Pat. No. 9,284,564), 1 hexanol (WO 2017/066498, U.S. Pat. No. 9,738,875), 1 octanol (WO 2017/066498, U.S. Pat. No. 9,738,875), chorismate-derived products (WO 2016/191625, U.S. Pat. No. 10,174,303), 3-hydroxybutyrate (WO 2017/066498, U.S. Pat. No. 9,738,875), 1,3-butanediol (WO 2017/066498, U.S. Pat. No. 9,738,875), 2-hydroxyisobutyrate or 2-hydroxyisobutyric acid (WO 2017/066498, U.S. Pat. No. 9,738,875), isobutylene (WO 2017/066498, U.S. Pat. No. 9,738,875), adipic acid (WO 2017/066498, U.S. Pat. No. 9,738,875), 1,3-hexanediol (WO 2017/066498, U.S. Pat. No. 9,738,875), 3-methyl-2-butanol (WO 2017/066498, U.S. Pat. No. 9,738,875), 2-buten-1-ol (WO 2017/066498, U.S. Pat. No. 9,738,875), isovalerate (WO 2017/066498, U.S. Pat. No. 9,738,875), isoamyl alcohol (WO 2017/066498, U.S. Pat. No. 9,738,875), and/or monoethylene glycol (WO 2019/126400, U.S. Pat. No. 11,555,209) in addition to 2-phenylethanol (WO 2021/188190, US 2021/0292732), fatty alcohols, fatty acids (e.g., omega-3 and/or omega-6 fatty acids), and/or other alkenes in addition to ethylene. In certain embodiments, microbial biomass itself may be considered a product. These products may be further converted to produce at least one component of diesel, jet fuel, sustainable aviation fuel (SAF) and/or gasoline. In certain embodiments, ethylene may be catalytically converted into another product, article, or any combination thereof. Additionally, the microbial biomass may be further processed to produce a single cell protein (SCP) by any method or combination of methods known in the art. In addition to one or more target chemical products, the microorganism of the disclosure may also produce pyruvate, acetate, ethanol, succinate, alpha-ketoglutarate, 3-hydroxybutyrate, and/or lactate. Additionally or alternatively, the microbial biomass may be further processed to produce a single cell protein (SCP) by any method or combination of methods known in the art. In addition to one or more target chemical products, the microorganism of the disclosure may also produce ethanol, acetate, and/or 2,3-butanediol. In another embodiment, the microorganism and methods of the disclosure improve the production of products, proteins, microbial biomass, or any combination thereof.

A “native product” is a product produced by a genetically unmodified microorganism. For example, polyhydroxyalkanoates are native products of Cupriavidus necator. A “non-native product” is a product that is produced by a genetically modified microorganism but is not produced by a genetically unmodified microorganism from which the genetically modified microorganism is derived. Ethylene is not known to be produced by any naturally-occurring microorganism, such that it is a non-native product of all microorganisms.

“Selectivity” refers to the ratio of the production of a target product to the production of all fermentation products produced by a microorganism. The microorganism of the disclosure may be engineered to produce products at a certain selectivity or at a minimum selectivity. In one embodiment, a target product, such as ethylene glycol, accounts for at least about 5%, 10%, 15%, 20%, 30%, 50%, or 75% of all fermentation products produced by the microorganism of the disclosure. In one embodiment, ethylene accounts for at least 10% of all fermentation products produced by the microorganism of the disclosure, such that the microorganism of the disclosure has a selectivity for ethylene glycol of at least 10%. In another embodiment, ethylene accounts for at least 30% of all fermentation products produced by the microorganism of the disclosure, such that the microorganism of the disclosure has a selectivity for ethylene of at least 30%.

At least one of the one or more fermentation products may be biomass produced by the culture. At least a portion of the microbial biomass may be converted to a single cell protein (SCP). At least a portion of the single cell protein may be utilized as a component of animal feed.

In one embodiment, the disclosure provides an animal feed comprising microbial biomass and at least one excipient, wherein the microbial biomass comprises a microorganism grown on a gaseous substrate comprising one or more of CO, CO2, and H2.

A “single cell protein” (SCP) refers to a microbial biomass that may be used in protein-rich human and/or animal feeds, often replacing conventional sources of protein supplementation such as soymeal or fishmeal. To produce a single cell protein, or other product, the process may comprise additional separation, processing, or treatments steps. For example, the method may comprise sterilizing the microbial biomass, centrifuging the microbial biomass, and/or drying the microbial biomass. In certain embodiments, the microbial biomass is dried using spray drying or paddle drying. The method may also comprise reducing the nucleic acid content of the microbial biomass using any method known in the art, since intake of a diet high in nucleic acid content may result in the accumulation of nucleic acid degradation products and/or gastrointestinal distress. The single cell protein may be suitable for feeding to animals, such as livestock or pets. In particular, the animal feed may be suitable for feeding to one or more beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents. The composition of the animal feed may be tailored to the nutritional requirements of different animals. Furthermore, the process may comprise blending or combining the microbial biomass with one or more excipients.

“Microbial biomass” refers biological material comprising microorganism cells. For example, microbial biomass may comprise or consist of a pure or substantially pure culture of a bacterium, archaea, virus, or fungus. When initially separated from a fermentation broth, microbial biomass generally contains a large amount of water. This water may be removed or reduced by drying or processing the microbial biomass.

The microbial biomass may comprise any of the components listed in this application but is not limited to the disclosures herein. Notably, the microbial biomass of an embodiment comprises 15% moisture (water) by weight. Accordingly, the values may refer to amounts of each component per amount of wet (i.e., non-dried) microbial biomass. Herein, the composition of the microbial biomass is described in terms of weight of a component per weight of wet (i.e., non-dried) microbial biomass. Of course, it is also possible to calculate the composition of the microbial biomass in terms of weight of a component per weight of dry microbial biomass.

The microbial biomass generally contains a large fraction of protein, such as more than 50% (50 g protein/100 g biomass), more than 60% (60 g protein/100 g biomass), more than 70% (70 g protein/100 g biomass), or more than 80% (80 g protein/100 g biomass) protein by weight. In a preferred embodiment, the microbial biomass comprises at least 72% (72 g protein/100 g biomass) protein by weight. The protein fraction comprises amino acids, including aspartic acid, alanine, arginine, cysteine, glutamic acid, glycine, histidine, isoleucine, leucine, lysine, methionine, phenylalanine, proline, serine, threonine, tyrosine, and/or valine. In particular, the microbial biomass may comprise more than 10 mg methionine/g biomass, more than 15 mg methionine/g biomass, more than 20 mg methionine/g biomass, or more than 25 mg methionine/g biomass. In a preferred embodiment, the microbial biomass comprises at least 17.6 mg methionine/g biomass.

The microbial biomass may contain a number of vitamins, including vitamins A (retinol), C, B1 (thiamine), B2 (riboflavin), B3 (niacin), B5 (pantothenic acid), and/or B6 (pyridoxine).

The microbial biomass may contain relatively small amounts of carbohydrates and fats. For example, the microbial biomass may comprise less than 15% (15 g carbohydrate/100 g biomass), less than 10% (10 g carbohydrate/100 g biomass), or less than 5% (5 g carbohydrate/100 g biomass) of carbohydrate by weight. For example, the microbial biomass may comprise less than 10% (10 g fat/100 g biomass), or less than 5% (5 g fat/100 g biomass), less than 2% (2 g fat/100 g biomass), or less than 1% (1 g fat/100 g biomass) of fat by weight.

The microorganism may classified based on functional characteristics. For example, the microorganism may be or may be derived from a C1-fixing microorganism, an aerobe, a hydrogen-oxidizing bacteria, a hydrogenotroph, an anaerobe, an acetogen, an ethanologen, and/or a carboxydotroph.

An “excipient” may refer to any substance that may be added to the microbial biomass to enhance or alter the form, properties, or nutritional content of the animal feed. For example, the excipient may comprise one or more of a carbohydrate, fiber, fat, protein, vitamin, mineral, water, flavour, sweetener, antioxidant, enzyme, preservative, probiotic, or antibiotic. In some embodiments, the excipient may be hay, straw, silage, grains, oils or fats, or other plant material. The excipient may be any feed ingredient identified in Chiba, Section 18: Diet Formulation and Common Feed Ingredients, Animal Nutrition Handbook, 3rd revision, pages 575-633, 2014.

A “biopolymer” refers to natural polymers produced by the cells of living organisms. In certain embodiments, the biopolymer is PHA. In certain embodiments, the biopolymer is PHB.

A “bioplastic” refers to plastic materials produced from renewable biomass sources. A bioplastic may be produced from renewable sources, such as vegetable fats and oils, corn starch, straw, woodchips, sawdust, or recycled food waste.

As used herein, the terms “protein-based bioplastic,” “protein bio-based plastic” and “protein biocomposite” can be used interchangeably. “Protein-based bioplastics” and “protein-based protein-based biofilms” refer to naturally-derived biodegradable polymers. Protein-based bioplastics and protein-based biofilms are largely composed of proteins. A “protein-based material” refers to a three-dimensional macromolecular network comprising hydrogen bonds, hydrophobic interactions, and disulphide bonds. See, e.g., Martinez, Journal of Food Engineering, 17: 247-254, 2013 and Pommet, Polymer, 44: 115-122, 2003. In preferred embodiments, the protein component of a protein-based bioplastic or protein-based biofilm is microbial biomass. Production of protein-based bioplastics and protein-based biofilms may require a step of protein denaturation by chemical, thermal, or pressure-induced methods. See, e.g., Mekonnen, Biocomposites: Design and Mechanical Performance, 2015. Production of protein-based bioplastics and protein-based biofilms may further require a step of isolating or fractionating the microbial biomass to produce a purified protein material.

The protein-based bioplastic or protein-based biofilm may be a blend of a protein, such as microbial biomass, with a plasticizer. As used herein, a “plasticizer” refers to a molecule having a low molecular weight and volatility. The plasticizer is used to modify the structure of a protein by reducing the intermolecular forces present in the protein and increasing polymeric chain mobility. See, e.g., Martinez, Journal of Food Engineering, 17: 247-254, 2013 and Gennadios, CRC Press, New York, 66-115, 2002. Non-limiting examples of plasticizers include water, glycerol, ethylene glycerol, propylene glycerol, palmitic acid, diethyl tartarate, dibutyl tartarate, 1,2-butanediol, 1,3-butanediol, polyethylene glycol (PEG), sorbitol, mantitoletc, dimethylaniline, diphenylamine, and 2,3-butanediol. See, e.g., Mekonnen, Biocomposites: Design and Mechanical Performance, 2015. In some embodiments, glycerol is used as a plasticizer. In some embodiments, 30% glycerol is used as a plasticizer. In some embodiments, 2,3-butanediol, which is a native product of Clostridium autoethanogenum, is used as a plasticizer.

In some embodiments, a plasticizer is introduced into a protein matrix by physicochemical methods, such as by a “casting” method. In this method, a chemical reactant is introduced to disrupt the disulphide bonds. See, e.g., Martinez, Journal of Food Engineering, 17: 247-254, 2013 and Gontard, J. Food Sci., 57: 190-196, 1993.

In some embodiments, a plasticizer is introduced into a protein matrix by thermoplastic processing. In this method, a protein and a plasticizer are mixed by a combination of heat and shear. This method may further require thermo-mechanical treatments, such as compression molding, thermomoulding, and extrusion. See, e.g., Martinez, Journal of Food Engineering, 17: 247-254, 2013 and Felix, Industrial Crops and Products, 79: 152-159, 2016.

In some embodiments, protein/plasticizer blends are prepared by a thermo-mechanical procedure, such as by mixing to obtain a dough-like material of appropriate consistency and homogeneity. The dough-like material is then processed by injection molding to produce a protein-based bioplastic or protein-based biofilm. See, e.g., Felix, Industrial Crops and Products, 79: 152-159, 2016.

In some embodiments, an additive is required to produce a protein-based bioplastic or a protein-based biofilm. For example, the additive may be a reducing agent, a cross-linking agent, a strengthener, a conductivity agent, a compatabilizing agent, or a water resistance agent. A non-limiting example of a reducing agent is sodium bisulfite. Non-limiting examples of cross-linking agents include glyoxal, L-cysteine, and formaldehyde. Non-limiting examples of strengtheners include bacterial cellulose nanofibers, pineapple leaf fibers, lignin, flax, jute, hemp, and sisal. A non-limiting example of a conductivity agent is a carbon nanotube material. Non-limiting examples of compatabilizing agents include malic anhydride and toluene diisocyanate. A non-limiting example of a water resistance agent is a polyphosphate material. In some embodiments, chemical modifications are used to improve water resistance. The chemical modification may be esterification with low molecular weight alcohols. See, e.g., Felix, Industrial Crops and Products, 79: 152-159, 2016 and Mekonnen, Biocomposites: Design and Mechanical Performance, 2015.

In some embodiments, a protein-based bioplastic or protein-based biofilm is produced by extrusion, wherein the microbial biomass is heated and pushed through an extrusion die.

In some embodiments, a protein-based bioplastic may be blended with fossil-derived plastics, but this is not a required step.

The protein-based bioplastics described herein may be used in packaging, bags, bottles, containers, disposable dishes, cutlery, plant pots, ground cover, baling hay, buttons, or buckles.

An advantage of the present disclosure is the solubility of microbial biomass in water. Although some research has been conducted related to use of plant proteins in protein-based bioplastics, few plant proteins are soluble in common solvents, and use of solvents or alkaline solutions increases cost and may be environmentally unfriendly. Perez, Food and Bioproducts Processing, 97: 100-108, 2016.

Herein, reference to an acid (e.g., acetic acid or 2-hydroxyisobutyric acid) should be taken to also include the corresponding salt (e.g., acetate or 2-hydroxyisobutyrate).

For the purposes of the present disclosure, the strain of C. necator may be a strain that grows autotrophically at up to 40° C. For example, the strain of C. necator may be the strain deposited at DSM34774. Additionally or alternatively, the strain of C. necator may have a H₂:CO₂ uptake ratio that is lower than the H₂:CO₂ uptake ratio of a wild type or naturally occurring strain of C. necator cultured under corresponding temperatures and conditions. In some embodiments, the strain of C. necator may comprise a full or partial deletion of a gene or locus encoding a membrane-bound hydrogenase, such as hoxKGXZ.

C. necator strains of present disclosure, including naturally occurring strains, temperature resistant strains, and other modified strains (e.g., strains modified to minimize the H₂:CO₂ uptake ratio while maximizing intracellular O₂ during autotrophic growth), provide several process advantages for the production of products.

A strain of C. necator can be modified to minimize the H₂:CO₂ uptake ratio by deleting all or a portion of, or otherwise reducing the expression of a membrane-bound hydrogenase system (e.g., the hoxKGZ locus). The hoxKGZ locus encodes a multi-subunit membrane-bound hydrogenase system in Cupriavidus necator, and deleting this locus does not inhibit C. necator autotrophic growth on a gas mixture composed of hydrogen, oxygen, and carbon dioxide. Without being bound by theory, this may result because while the soluble hydrogenase system is required for autotrophic growth, the membrane-bound hydrogenase system wastefully oxidizes hydrogen gas, increasing the culture's H₂:CO₂ uptake ratio without benefiting growth. By deleting or reducing expression of the hoxKGZ locus, growth is not only permitted autotrophically, but the H₂:CO₂ uptake ratio is also reduced while increasing the intracellular O₂ availability to improve O₂-dependent product synthesis.

### III. CULTURE AND FERMENTATION OF THE MICROORGANISM

Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. Suitable media are well known in the art.

The culture/fermentation should desirably be carried out under appropriate conditions for production of desired products. If necessary, the culture/fermentation is performed under aerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting.

Operating a bioreactor at elevated pressures allows for an increased rate of gas mass transfer from the gas phase to the liquid phase. Accordingly, it is generally preferable to perform the culture/fermentation at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, it is preferable to operate the fermentation at a pressure higher than atmospheric pressure. Also, since a given gas conversion rate is in part a function of substrate retention time and achieving a desired retention time in turn dictates the required volume of a bioreactor, the use of pressurized systems can greatly reduce the volume of the bioreactor required, and consequently the capital cost of the fermentation equipment.

A “sparger” may comprise a device to introduce gas into a liquid, injected as bubbles, to agitate it or to dissolve the gas in the liquid. Example spargers may include orifice spargers, sintered spargers, and drilled pipe spargers. In certain configurations drilled pipe spargers may be mounted horizontally. In other examples, spargers may be mounted vertically or horizontally. In some examples, the sparger may be a perforated plate or ring, sintered glass, sintered steel, porous rubber pipe, porous metal pipe, porous ceramic or stainless steel, drilled pipe, stainless steel drilled pipe, polymeric drilled pipe, etc. The sparger may be of various grades (porosities) or may include certain sized orifices to produce a specific sized bubble or range of bubble sizes.

A “vessel”, “reaction vessel”, or “column” may be a vessel or container in which one or more gas and liquid streams, or flows may be introduced for bubble generation and/or fine bubble generation, and for subsequent gas-liquid contacting, gas-absorption, biological or chemical reaction, or surface-active material adsorption. In a reaction vessel, the gas and liquid phases may flow in the vertical directions. In a reaction vessel, larger bubbles from a sparger, having a buoyancy force larger than the drag force imparted by the liquid, may rise upwards. Smaller fine bubbles, having a buoyancy force less than or equal to the drag force imparted by the liquid, may flow downward with the liquid, as described by the systems and methods disclosed herein. A column or reaction vessel may not be restricted to any specific aspect (height to diameter) ratio. A column or reaction vessel may also not be restricted to any specific material and can be constructed from any material suitable to the process such as stainless steel, PVC, carbon steel, or polymeric material. A column or reaction vessel may contain internal components such as one or more static mixers that are common in biological and chemical engineering processing. A reaction vessel may also consist of external or internal heating or cooling elements such as water jackets, heat exchangers, or cooling coils. The reaction vessel may also be in fluid contact with one or more pumps to circulate liquid, bubbles, fine bubbles, and or one or more fluids of the system.

A “perforated plate” or “plate” may comprise a plate or similar arrangement designed to facilitate the introduction of liquid or additional liquid into the vessel that may be in the form of multiple liquid jets (i.e., accelerated liquid flow). The perforated plate may have a plurality of pores or orifices evenly or unevenly distributed across the plate that allow the flow of liquid from a top of the plate to the bottom of the plate. In some examples, the orifices may be spherical-shaped, rectangular-shaped, hexagonal prism-shaped, conical-shaped, pentagonal prism-shaped, cylindrical-shaped, frustoconical-shaped, or round-shaped. In other examples, the plate may comprise one or more nozzles adapted to generate liquid jets which flow into the column. The plate may also contain channels in any distribution or alignment where such channels are adapted to receive liquid and facilitate flow through into the reaction vessel. The plate may be made of stainless steel with a predefined number of laser-burnt, machined, or drilled pores or orifices. The specific orifice size may depend upon the required fine bubble size and required liquid, fine bubble, and/or fluid velocities. A specific orifice shape may be required to achieve the proper liquid acceleration and velocity from the plate to break or shear the sparger bubbles into the desired fine bubble size, and to create enough overall fluid downflow to carry the fine bubbles and liquid downward in the reaction vessel. The shape of the orifice may also impact ease of manufacturing and related costs. According to one embodiment, a straight orifice may be optimal due to ease of manufacture.

The systems and methods as disclosed herein, employ, within a vessel, multiple liquid jets or portions of accelerated liquid flow generated using the perforated plate to accelerate liquid and break bubbles into smaller fine bubbles having a greater superficial surface area than the original bubbles. The original bubbles are initially generated by injecting gas with a sparger positioned entirely within the reaction vessel. In one example, original bubbles injected into liquid from a sparger may have a diameter of about 2 mm to about 20 mm. In another example, original bubbles injected into liquid from a sparger may have a diameter of about 5 mm to about 15 mm. In other examples, original bubbles injected into liquid from a sparger may have a diameter of about 7 mm to about 13 mm. Upon injection, the original bubbles subsequently migrate upwards through the liquid and encounter the multiple liquid jets or portions of accelerated liquid flow which breaks the original bubbles into fine bubbles. The resulting fine bubbles and liquid flow down the reactor vessel in the downward fluid flow. The fine bubbles of substrate provide a carbon source and optionally an energy source to the microbes which then produce one or more desired products. The spargers are positioned within the vessel to create a first zone for the original bubbles to rise within the vessel, and to create a second zone for the accelerated liquid to break the original bubbles into fine bubbles and for fluid to flow through the vessel, where the fluid comprises the accelerated portion of the liquid and fine bubbles.

Due to the nature of the multi-phase system, one approach to maximizing product generation is to increase gas to liquid mass transfer. The more gas substrate transferred to a reaction liquid, the greater the desired product generated. The smaller fine bubbles of the present disclosure provide an increased superficial surface area resulting in an increased gas to liquid mass transfer rates overcoming known solubility issues. Additionally, the downflow reactor systems disclosed herein are effective to increase the residence time of the fine bubbles. The increased time that the fine bubbles remain in the reaction liquid generally provides increased amounts of reaction product generated, as well as greater surface areas in contact with the microbes. As such, the systems and methods disclosed herein improve over previous systems by generating fine bubbles that maximize gas to liquid superficial surface areas leading to high gas to liquid mass transfer rates. Further, the systems and methods disclosed herein provide superficial gas and liquid velocities not achieved by the previous systems and methods resulting in the generation of fine bubbles with high gas phase residence time resulting in the efficient creation of chemical and biological reaction products.

In certain embodiments, the fermentation is performed in the absence of light or in the presence of an amount of light insufficient to meet the energetic requirements of photosynthetic microorganisms. In certain embodiments, the microorganism of the disclosure is a non-photosynthetic microorganism.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells are preferably returned to the bioreactor. The cell-free permeate remaining after target products have been removed is also preferably returned to the bioreactor. Additional nutrients (such as B vitamins) may be added to the cell-free permeate to replenish the medium before it is returned to the bioreactor. Purification techniques may include affinity tag purification (e.g. His, Twin-Strep, and FLAG), bead-based systems, a tip-based approach, and FPLC system for larger scale, automated purifications. Purification methods that do not rely on affinity tags (e.g. salting out, ion exchange, and size exclusion) are also disclosed.

In some embodiments, the produced chemical product may be isolated and enriched, including purified, using any suitable separation and/or purification technique known in the art. In an embodiment, the produced chemical product is gaseous. In one embodiment, the chemical product is a liquid. In an embodiment, a gaseous chemical product may pass a filter, a gas separation membrane, a gas purifier, or any combination thereof. In one embodiment, the chemical product is separated by an absorbent column. In another embodiment, the chemical product is stored in one or more cylinders after separation. In one embodiment, the chemical product is integrated into an infrastructure or process of an oil, gas, refinery, petrochemical operation, or any combination thereof. The infrastructure or process may be existing or new. In an embodiment, the gas fermentation product is integrated into oil and gas production, transportation and refining, and/or chemical complexes. In another embodiment, the source of the feedstock is from an oil, gas, refinery, petrochemical operation, or any combination thereof. In an embodiment, the gas fermentation product is integrated into an infrastructure or process of an oil, gas, refinery, petrochemical operation, or any combination thereof, and the source of the feedstock is from an oil, gas, refinery, petrochemical operation, or any combination thereof.

In some embodiments, distillation may be employed to purify a product gas. In an embodiment, gas-liquid extraction may be employed. In an embodiment, a liquid product isolation may also be enriched via extraction using an organic phase. In another embodiment, purification may involve other standard techniques selected from ultrafiltration, one or more chromatographic techniques, or any combination thereof.

The method of the disclosure may further comprise separating a gas fermentation product from the fermentation broth. The gas fermentation product may be separated or purified from a fermentation broth using any method or combination of methods known in the art, including, for example, distillation, simulated moving bed processes, membrane treatment, evaporation, pervaporation, gas stripping, phase separation, ion exchange, or extractive fermentation, including for example, liquid-liquid extraction. As described in U.S. Pat. No. 2,769,321, the disclosure of which is incorporated by reference in its entirety herein, ethylene may be separated according to the method or combination of methods known in the art. In one embodiment, the ethylene produced is harvested from the bioreactor culture vessel.

In one embodiment, the gas fermentation product may be concentrated from the fermentation broth using reverse osmosis and/or pervaporation (U.S. Pat. No. 5,552,023). Water may be removed by distillation and the bottoms (containing a high proportion of gas fermentation product) may then be recovered using distillation or vacuum distillation to produce a high purity stream. Alternatively, with or without concentration by reverse osmosis and/or pervaporation, the gas fermentation product may be further purified by reactive distillation with an aldehyde (Atul, Chem Eng Sci, 59: 2881-2890, 2004) or azeotropic distillation using a hydrocarbon (U.S. Pat. No. 2,218,234). In another approach, the gas fermentation product may be trapped on an activated carbon or polymer absorbent from aqueous solution (with or without reverse osmosis and/or pervaporation) and recovered using a low boiling organic solvent (Chinn, Recovery of Glycols, Sugars, and Related Multiple—OH Compounds from Dilute-Aqueous Solution by Regenerable Adsorption onto Activated Carbons, University of California Berkeley, 1999). The gas fermentation product can then be recovered from the organic solvent by distillation. In certain embodiments, the gas fermentation product is recovered from the fermentation broth by continuously removing a portion of the broth from the bioreactor, separating microbial cells from the broth (conveniently by filtration), and recovering the gas fermentation product from the broth. Co-products, such as alcohols or acids may also be separated or purified from the broth. Alcohols may be recovered, for example, by distillation. Acids may be recovered, for example, by adsorption on activated charcoal. Separated microbial cells may be returned to the bioreactor in certain embodiments. Further, separated microbial cells may be recycled to the bioreactor in some embodiments. The cell-free permeate remaining after target products have been removed is also preferably returned to the bioreactor, in whole or in part. Additional nutrients (such as B vitamins) may be added to the cell-free permeate to replenish the medium before it is returned to the bioreactor.

Recovery of diols from aqueous media has been demonstrated a number of ways. Simulated moving bed (SMB) technology has been used to recover 2,3-butaendiol from an aqueous mixture of ethanol and associated oxygenates (U.S. Pat. No. 8,658,845). Reactive separation has also been demonstrated for effective diol recovery. In some embodiments, recovery of ethylene glycol is conducted by reaction of the diol-containing stream with aldehydes, fractionation and regeneration of the diol, final fractionation to recover a concentrated diol stream. See, e.g., U.S. Pat. No. 7,951,980.

In one embodiment, the method comprises recovering ethylene produced as disclosed above. In one embodiment, the method further comprises converting or using ethylene in the production of one or more chemical products following recovery of ethylene.

Ethylene is a high value gaseous compound which is widely used in industry. In an embodiment, ethylene may be used as an anaesthetic or as a fruit ripening agent, as well as in the production of a number of other chemical products. In some embodiments, ethylene may be used to produce polyethylene and other polymers, such as styrene, polystyrene, ethylene oxide, ethylene dichloride, ethylene dibromide, ethyl chloride and ethylbenzene. Ethylene oxide is, for example, a key raw material in the production of surfactants and detergents and in the production of ethylene glycol, which is used in the automotive industry as an antifreeze product. In one embodiment directed to ethylene dichloride, ethylene dibromide, and ethyl chloride may be used to produce products such as polyvinyl chloride, trichloroethylene, perchloroethylene, methyl chloroform, polyvinylidene chloride and copolymers, and ethyl bromide. In an embodiment, ethylbenzene is a precursor to styrene, which is used in the production of polystyrene (used as an insulation product) and styrene-butadiene (which is rubber suitable for use in tires and footwear). In another embodiment, a product is an ethylene propylene diene monomer (EPDM) rubber, an ethylene propylene (EPR/EPM) rubber, or any combination thereof.

It should be appreciated that the methods of the disclosure may be integrated or linked with one or more methods for the production of downstream chemical products from ethylene. In some embodiments, the methods of the disclosure may feed ethylene directly or indirectly to chemical processes or reactions sufficient for the conversion or production of other useful chemical products.

In some embodiments, ethylene is converted into hydrocarbon liquid fuels. In an embodiment, ethylene is oligomerized over a catalyst to selectively produce target products selected from gasoline, condensate, aromatics, heavy oil diluents, distillates, or any combination thereof. In other embodiments, the distillates are selected from diesel, jet fuel, sustainable aviation fuel (SAF), or any combination thereof.

In one embodiment, ethylene oligomerization is utilized towards desirable products. In an embodiment, oligomerization of ethylene may be catalyzed by a homogeneous catalyst, heterogeneous catalyst, or any combination thereof and having transition metals as active sites. In some embodiments, ethylene is further converted into long chain hydrocarbons by oligomerization. In other embodiments, straight chain olefins are the main product from ethylene oligomerization. In some embodiments, alpha olefins are the main product from ethylene oligomerization. In an embodiment, olefins are subjected to upgrading processes. In some embodiments, the upgrading process of olefins is hydrogenation. In an embodiment, olefins are subjected to olefin conversion technology. In some embodiments, the ethylene is incorporated in or converted to sustainable aviation fuel (SAF). In one embodiment, ethylene is interconverted to propylene, 2-butenes, or any combination thereof. In an embodiment, propylene is converted to polypropylene.

As a raw material, ethylene can used in the manufacture of polymers such as polyethylene (PE), polyethylene terephthalate (PET) and polyvinyl chloride (PVC) as well as fibres and other organic chemicals. These products are used in a wide variety of industrial and consumer markets such as the packaging, transportation, electrical/electronic, textile and construction industries as well as consumer chemicals, coatings and adhesives.

Ethylene can be chlorinated to ethylene dichloride (EDC) and can then be cracked to make vinyl chloride monomer (VCM). Nearly all VCM is used to make polyvinyl chloride which has its main applications in the construction industry.

Other ethylene derivatives include alpha olefins which are used in Linear low-density polyethylene (LLDPE) production, detergent alcohols and plasticizer alcohols; vinyl acetate monomer (VAM) which is used in adhesives, paints, paper coatings and barrier resins; and industrial ethanol which is used as a solvent or in the manufacture of chemical intermediates such as ethyl acetate and ethyl acrylate. Ethylene may be converted into ethylene-vinyl acetate (EVA), or poly(ethylene-vinyl acetate) (PEVA). EVA may be converted to thermoplastics materials. EVA may be incorporated in or used to make hot melt adhesives, hot glue sticks, soccer cleats, plastic wraps, craft foam sheets, and foam stickers. EVA may be incorporated in or used to make a drug delivery device. In some embodiments, EVA may be used to make foam. In one embodiment, EVA foam is used as padding in equipment for sports, including ski boots, bicycle saddles, hockey pads, boxing and mixed-martial-arts gloves and helmets, wakeboard boots, waterski boots, fishing rods and fishing-reel handles. In some embodiments EVA foam is used as a shock absorber in sports shoes. EVA may be used as EVA-based compression-moulded foam. EVA may be incorporated in or used to make floats for commercial fishing gear and floating eyewear. EVA can be incorporated or used to make encapsulation material for crystalline silicon solar cells. In some embodiments, EVA may be incorporated in or used to make slippers, sandals, fishing rods, substitute for cork, packaging, textile, bookbinding, bonding plastic films, metal surfaces, coated paper, redispersible powders in plasters and cement renders, and coating formulations in interior water-borne paints. EVA may undergo hydrolysis to provide ethylene vinyl alcohol (EVOH) copolymers. EVA may be used in orthotics, surfboard and skimboard traction pads, car mats, artificial flowers, a cold flow improver for diesel fuel, as a separator in HEPA filters, thermoplastic mouthguards, for conditioning and waterproofing leather, in nicotine transdermal patches, and plastic model kit parts.

Ethylene may further be used as a monomer base for the production of various polyethylene oligomers by way of coordination polymerization using metal chloride or metal oxide catalysts. The most common catalysts consist of titanium (III) chloride, the so-called Ziegler-Natta catalysts. Another common catalyst is the Phillips catalyst, prepared by depositing chromium (VI) oxide on silica.

Polyethylene oligomers so produced may be classified according to its density and branching. Further, mechanical properties depend significantly on variables such as the extent and type of branching, the crystal structure, and the molecular weight. There are several types of polyethylene which may be generated from ethylene, including, but not limited to:
Ultra-high-molecular-weight polyethylene (UHMWPE);
Ultra-low-molecular-weight polyethylene (ULMWPE or PE-WAX);
High-molecular-weight polyethylene (HMWPE);
High-density polyethylene (HDPE);
High-density cross-linked polyethylene (HDXLPE);
Cross-linked polyethylene (PEX or XLPE);
Medium-density polyethylene (MDPE);
Linear low-density polyethylene (LLDPE);
Low-density polyethylene (LDPE);
Very-low-density polyethylene (VLDPE); and
Chlorinated polyethylene (CPE).

Low density polyethylene (LDPE) and linear low-density polyethylene (LLDPE) mainly go into film applications such as food and non-food packaging, shrink and stretch film, and non-packaging uses. High density polyethylene (HDPE) is used primarily in blow molding and injection molding applications such as containers, drums, household goods, caps and pallets. HDPE can also be extruded into pipes for water, gas and irrigation, and film for refuse sacks, carrier bags and industrial lining.

According to one embodiment, the ethylene formed from the disclosure described above may be converted to ethylene oxide via direct oxidation according to the following formula:

C₂H₄+O₂→C₂H₄O

The ethylene oxide produced thereby is a key chemical intermediate in a number of commercially important processes including the manufacture of monoethylene glycol. Other EO derivatives include ethoxylates (for use in shampoo, kitchen cleaners, etc.), glycol ethers (solvents, fuels, etc.) and ethanolamines (surfactants, personal care products, etc.).

According to one embodiment of the disclosure, the ethylene oxide produced as described above may be used to produce commercial quantities of monoethylene glycol by way of the formula:

(CH₂CH₂)O+H₂O→HOCH₂CH₂OH

According to another embodiment, the claimed microorganism can be modified in order to directly produce monoethylene glycol. As described in WO 2019/126400, the disclosure of which is incorporated by reference in its entirety herein, the microorganism further comprises one or more of an enzymes capable of converting acetyl-CoA to pyruvate; an enzyme capable of converting pyruvate to oxaloacetate; an enzyme capable of converting pyruvate to malate; an enzyme capable of converting pyruvate to phosphoenolpyruvate; an enzyme capable of converting oxaloacetate to citryl-CoA; an enzyme capable of converting citryl-CoA to citrate; an enzyme capable of converting citrate to aconitate and aconitate to iso-citrate; an enzyme capable of converting phosphoenolpyruvate to oxaloacetate; an enzyme capable of converting phosphoenolpyruvate to 2-phospho-D-glycerate; an enzyme capable of converting 2-phospho-D-glycerate to 3-phospho-D-glycerate; an enzyme capable of converting 3-phospho-D-glycerate to 3-phosphonooxypyruvate; an enzyme capable of converting 3-phosphonooxypyruvate to 3-phospho-L-serine; an enzyme capable of converting 3-phospho-L-serine to serine; an enzyme capable of converting serine to glycine; an enzyme capable of converting 5,10-methylenetetrahydrofolate to glycine; an enzyme capable of converting serine to hydroxypyruvate; an enzyme capable of converting D-glycerate to hydroxypyruvate; an enzyme capable of converting malate to glyoxylate; an enzyme capable of converting glyoxylate to glycolate; an enzyme capable of converting hydroxypyruvate to glycolaldehyde; and/or an enzyme capable of converting glycolaldehyde to ethylene glycol.

In one embodiment, the microorganism comprises one or more of a heterologous enzyme capable of converting oxaloacetate to citrate; a heterologous enzyme capable of converting glycine to glyoxylate; a heterologous enzyme capable of converting iso-citrate to glyoxylate; a heterologous enzyme capable of converting glycolate to glycolaldehyde; or any combination thereof. In some embodiments, wherein the heterologous enzyme capable of converting oxaloacetate to citrate is a citrate [Si]-synthase [2.3.3.1], an ATP citrate synthase [2.3.3.8]; or a citrate (Re)-synthase [2.3.3.3]; the heterologous enzyme capable of converting glycine to glyoxylate is an alanine-glyoxylate transaminase [2.6.1.44], a serine-glyoxylate transaminase [2.6.1.45], a serine-pyruvate transaminase [2.6.1.51], a glycine-oxaloacetate transaminase [2.6.1.35], a glycine transaminase [2.6.1.4], a glycine dehydrogenase [1.4.1.10], an alanine dehydrogenase [1.4.1.1], or a glycine dehydrogenase [1.4.2.1]; the heterologous enzyme capable of converting iso-citrate to glyoxylate is an isocitrate lyase [4.1.3.1]; the heterologous enzyme capable of converting glycolate to glycolaldehyde is a glycolaldehyde dehydrogenase [1.2.1.21], a lactaldehyde dehydrogenase [1.2.1.22], a succinate-semialdehyde dehydrogenase [1.2.1.24], a 2,5-dioxovalerate dehydrogenase [1.2.1.26], an aldehyde dehydrogenase [1.2.1.3/4/5], a betaine-aldehyde dehydrogenase [1.2.1.8], or an aldehyde ferredoxin oxidoreductase [1.2.7.5]; or any combination thereof.

Monoethylene glycol produced according to either of the described methods may be used as a component of a variety of products including as a raw material to make polyester fibers for textile applications, including nonwovens, cover stock for diapers, building materials, construction materials, road-building fabrics, filters, fiberfill, felts, transportation upholstery, paper and tape reinforcement, tents, rope and cordage, sails, fish netting, seatbelts, laundry bags, synthetic artery replacements, carpets, rugs, apparel, sheets and pillowcases, towels, curtains, draperies, bed ticking, and blankets.

MEG may be used on its own as a liquid coolant, antifreeze, preservative, dehydrating agent, drilling fluid or any combination thereof. The MEG produced may also be used to produce secondary products such as polyester resins for use in insulation materials, polyester film, de-icing fluids, heat transfer fluids, automotive antifreeze and other liquid coolants, preservatives, dehydrating agents, drilling fluids, water-based adhesives, latex paints and asphalt emulsions, electrolytic capacitors, paper, and synthetic leather.

Importantly, the monoethylene glycol produced may be converted to the polyester resin polyethylene terephthalate (“PET”) according to one of two major processes. The first process comprises transesterification of the monoethylene glycol utilizing dimethyl terephthalate, according to the following two-step process:

C₆H₄(CO₂CH₃)₂+2HOCH₂CH₂OH→C₆H₄(CO₂CH₂CH₂OH)₂+2CH₃OH First step

n C₆H₄(CO₂CH₂CH₂OH)₂→[(CO)C₆H₄(CO₂CH₂CH₂O)]ₙ+n HOCH₂CH₂OH Second step

Alternatively, the monoethylene glycol can be the subject of an esterification reaction utilizing terephthalic acid according to the following reaction:

n C₆H₄(CO₂H)₂+n HOCH₂CH₂OH→[(CO)C₆H₄(CO₂CH₂CH₂O)]ₙ+2n H₂O

The polyethylene terephthalate produced according to either the transesterification or esterification of monoethylene glycol has significant applicability to numerous packaging applications such as jars and, in particular, in the production of bottles, including plastic bottles. It can also be used in the production of high-strength textile fibers such as Dacron, as part of durable-press blends with other fibers such as rayon, wool, and cotton, for fiber fillings used in insulated clothing, furniture, and pillows, in artificial silk, as carpet fiber, automobile tire yarns, conveyor belts and drive belts, reinforcement for fire and garden hoses, seat belts, nonwoven fabrics for stabilizing drainage ditches, culverts, and railroad beds, and nonwovens for use as diaper topsheets, and disposable medical garments.

At a higher molecular weight, PET can be made into a high-strength plastic that can be shaped by all the common methods employed with other thermoplastics. Magnetic recording tape and photographic film are produced by extrusion of PET film. Molten PET can be blow-molded into transparent containers of high strength and rigidity that are also virtually impermeable to gas and liquid. In this form, PET has become widely used in bottles, especially plastic bottles, and in jars.

The disclosure provides compositions comprising ethylene glycol produced by the microorganisms and according to the methods described herein. For example, the composition comprising ethylene glycol may be an antifreeze, preservative, dehydrating agent, or drilling fluid.

The disclosure also provides polymers comprising ethylene glycol produced by the microorganisms and according to the methods described herein. Such polymers may be, for example, homopolymers such as polyethylene glycol or copolymers such as polyethylene terephthalate. Methods for the synthesis of these polymers are well-known in the art. See, e.g., Herzberger et al., Chem Rev., 116(4): 2170-2243 (2016) and Xiao et al., Ind Eng Chem Res. 54(22): 5862-5869 (2015).

The disclosure further provides polyethylene glycol conjugates. In some embodiments, polyethylene glycol (PEG) conjugates include PEG conjugated to a biopharmaceutical, proteins, antibodies, anticancer drugs, or any combination thereof. In other embodiments, the PEG conjugate is diethyl terephthalate (DET). In some embodiments, the PEG conjugate is dimethoxyethane.

The disclosure further provides compositions comprising polymers comprising ethylene glycol produced by the microorganisms and according to the methods described herein. For example, the composition may be a fiber, resin, film, or plastic.

In one embodiment, ethanol or ethyl alcohol produced according to the method of the disclosure may be used in numerous product applications, including antiseptic hand rubs (WO 2014/100851), therapeutic treatments for methylene glycol and methanol poisoning (WO 2006/088491), as a pharmaceutical solvent for applications such as pain medication (WO 2011/034887) and oral hygiene products (U.S. Pat. No. 6,811,769), as well as an antimicrobial preservative (U.S. Patent Application No. 2013/0230609), engine fuel (U.S. Pat. No. 1,128,549), rocket fuel (U.S. Pat. No. 3,020,708), plastics, fuel cells (U.S. Pat. No. 2,405,986), home fireplace fuels (U.S. Pat. No. 4,692,168), as an industrial chemical precursor (U.S. Pat. No. 3,102,875), cannabis solvent (WO 2015/073854), as a winterization extraction solvent (WO 2017/161387), as a paint masking product (WO 1992/008555), as a paint or tincture (U.S. Pat. No. 1,408,091), purification and extraction of DNA and RNA (WO 1997/010331), and as a cooling bath for various chemical reactions (U.S. Pat. No. 2,099,090). In addition to the foregoing, the ethanol generated by the disclosed method may be used in any other application for which ethanol might otherwise be applicable.

In an additional embodiment, isopropanol or isopropyl alcohol (IPA) produced according to the method may be used in numerous product applications, including either in isolation or as a feedstock for the production for more complex products. Isopropanol may also be used in solvents for cosmetics and personal care products, de-icers, paints and resins, food, inks, adhesives, and pharmaceuticals, including products such as medicinal tablets as well as disinfectants, sterilisers and skin creams.

The IPA produced may be used in the extraction and purification of natural products such as vegetable and animal oil and fats. Other applications include its use as a cleaning and drying agent in the manufacture of electronic parts and metals, and as an aerosol solvent in medical and veterinary products. It can also be used as a coolant in beer manufacture, a coupling agent, a polymerisation modifier, a de-icing agent and a preservative.

Alternatively, the IPA produced according to the method of the disclosure may be used to manufacture additional useful compounds, including plastics, derivative ketones such as methyl isobutyl ketone (MIBK), isopropylamines and isopropyl esters. Still further, the IPA may be converted to propylene according to the following formula:

CH₃CH₂CH₂OH→CH₃—CH═CH₂

The propylene produced may be used as a monomer base for the production of various polypropylene oligomers by way of chain-growth polymerization via either gas-phase or bulk reactor systems. The most common catalysts consist of titanium (III) chloride, the so-called Ziegler-Natta catalysts and metallocene catalysts.

Polypropylene oligomers so produced may be classified according to tacticity and can be formed into numerous products by either extrusion or molding of polypropylene pellets, including piping products, heat-resistant articles such as kettles and food containers, disposable bottles (including plastic bottles), clear bags, flooring such as rugs and mats, ropes, adhesive stickers, as well as foam polypropylene which can be used in building materials. Polypropylene may also be used for hydrophilic clothing and medical dressings.

In some embodiments ethylene is used to produce polyethylene: a common plastic used in a variety of consumer products such as plastic bags, plastic films, geomembranes, containers including bottles, etc.

In an embodiment ethylene is used to make ethylene glycol: a raw material in the manufacture of polyester fibers for clothes, upholstery, carpet, and pillows. In another embodiment ethylene used in the production of antifreeze in cooling and heating systems.

In another embodiment, ethylene is used in ethylene oxide: used to make other chemicals that are used in making products such as detergents, thickeners, solvents, plastics, and various organic chemicals. In some embodiments ethylene oxide is used as a sterilizing agent for medical equipment and a fumigating agent.

In some embodiments ethylene is used in vinyl acetate: used to make other chemicals that are used in paints, adhesives, paper coatings, and textiles.

In other embodiments, ethylene is used in ethylene dichloride: for the production of vinyl chloride, which is used to make polyvinyl chloride (PVC). PVC is used to make a variety of plastic and vinyl products including pipes, wire and cable coatings, and packaging materials.

In some embodiments, ethylene is used in aluminum alkyls: used as catalysts to increase the efficiency of making ethylene and other chemicals.

In some embodiments, ethylene is used in Ethylene Propylene Rubber (EPR): used in electrical insulation, roofing membrane, radiator hoses in vehicles, and waterproofing sheets.

In other embodiments, ethylene is used in agriculture: as a plant hormone and is used in agriculture to force the ripening of fruits.

In some embodiments, ethylene is used to make styrene: which is then used to make polystyrene. Polystyrene is used in various consumer products like disposable cutlery, CD and DVD cases, and insulation material.

In embodiments, ethylene is used to produce alpha olefins: used as co-monomers in the production of polyethylene, as well as in the production of detergents and lubricants.

In one embodiment, ethylene is used to produce butadiene. In some embodiments the butadiene is used in rubber tires.

In an embodiment, a method for the continuous production of ethylene, the process comprising: passing a gaseous substrate to a bioreactor containing a culture of a recombinant C1-fixing microorganism capable of producing ethylene in a culture medium such that the microorganism converts the gaseous substrate to ethylene; and recovering the ethylene from the bioreactor.

In other embodiments, converting the ethylene into a component used to manufacture tires. In an embodiment, the ethylene is converted into a component used in tire threads.

The method according to an embodiment, wherein the tires are end-of-life tires.

The method according to an embodiment, wherein the gaseous substrate is derived from a process comprising tires.

The method according to an embodiment, wherein the gaseous substrate is derived from a product circularity process or a sustainable chemical process.

The method according to an embodiment, further comprising converting the ethylene to a component used to manufacture new tires.

The method according to an embodiment, comprising resin components selected from ethylene and other olefins bonded to synthetic components selected from butadiene and isoprene to form hybrid polymers used to manufacture tires.

One embodiment is directed to a method for producing a polymer from a gaseous substrate comprising a first gas fermentation process produces at least one first product selected from butadiene, isoprene, conjugated dienes, or any combination thereof and a second gas fermentation process produces at least one second product selected from ethylene and olefins, or any combination thereof, and wherein the at least one first product and at least one second product are copolymerized to form a polymer.

The method according to an embodiment, wherein the first gas fermentation process and the second gas fermentation process are run in parallel.

The method according to an embodiment, wherein the first gas fermentation process and the second gas fermentation process are both run continuously.

The method according to an embodiment, comprising a first gas fermentation process produces rubber component and a second gas fermentation process produces a resin component, and wherein the rubber component and resin component are copolymerized to form a polymer.

The method according to an embodiment, wherein the rubber component and resin component are copolymerized by a suitable polymerization catalyst.

The method according to an embodiment, wherein the rubber component is selected from butadiene, isoprene, conjugated dienes, or any combination thereof.

The method according to an embodiment, wherein the resin component is selected from ethylene, olefins, or any combination thereof.

The method according to an embodiment, wherein the suitable polymerization catalyst further comprises another component contained in a general polymerization catalyst composition containing a metallocene complex.

The method according to an embodiment, wherein the metallocene complex is a complex compound having one or more cyclopentadienyl groups or derivative cyclopentadienyl groups bonded to a central metal.

The method according to an embodiment, wherein the central metal is selected from a lanthanoid element, scandium, yttrium, or any combination thereof.

The method according to an embodiment, wherein the central metal is selected from samarium (Sm), neodymium (Nd), praseodymium (Pr), gadolinium (Gd), cerium (Ce), holmium (Ho), scandium (Sc), and yttrium (Y).

The method according to an embodiment, further comprising converting the polymer into a tire.

One embodiment for the circular production of tires from a gaseous substrate is directed to a first gas fermentation process to produce at least one first product selected from butadiene, isoprene, conjugated dienes, or any combination thereof; and a second gas fermentation process to produce at least one second product selected from ethylene and olefins, or any combination thereof, wherein the at least one first product and at least one second product are copolymerized to form a polymer, and wherein the substrate is derived from a process comprising tires.

The method according to an embodiment, wherein the substrate is derived from a process comprising end-of-life tires.

One embodiment is directed to a method for the circular production of tires, the method comprising: 1) passing a gaseous substrate to a first bioreactor containing a culture of a recombinant C1-fixing microorganism capable of producing at least one first product selected from butadiene, isoprene, conjugated dienes, or any combination thereof in a culture medium such that the microorganism converts the gaseous substrate to the at least one first product; and recovering the at least one first product from the bioreactor; 2) passing a gaseous substrate to a second bioreactor containing a culture of a recombinant C1-fixing microorganism capable of producing at least one second product selected from ethylene and olefins, or any combination thereof in a culture medium such that the microorganism converts the gaseous substrate to the at least one second product; and recovering the at least one second product from the bioreactor; 3) polymerizing the at least one first product with the at least one second product in the presence of a suitable polymerization catalyst to form a hybrid polymer; and 4) converting the hybrid polymer into a tire.

The method according to an embodiment, wherein the suitable polymerization catalyst further comprises another component contained in a general polymerization catalyst composition containing a metallocene complex.

The method according to an embodiment, wherein the metallocene complex is a complex compound having one or more cyclopentadienyl groups or derivative cyclopentadienyl groups bonded to a central metal.

The method according to an embodiment, wherein the central metal is selected from a lanthanoid element, scandium, yttrium, or any combination thereof.

The method according to an embodiment, wherein the central metal is selected from samarium (Sm), neodymium (Nd), praseodymium (Pr), gadolinium (Gd), cerium (Ce), holmium (Ho), scandium (Sc), and yttrium (Y).

The method according to an embodiment, wherein the first bioreactor and the second bioreactor are run in parallel.

The method according to an embodiment, wherein both the first bioreactor and the second bioreactor are continuously operated.

The method according to an embodiment, wherein the substrates are derived from a process comprising end-of-life tires.

The method according to an embodiment further comprising converting the isoprenoid into a product selected from synthetic rubber, block polymers containing styrene, thermoplastic rubbers, pressure-sensitive or thermosetting adhesives, butyl rubber, terpenes selected from citral, linalool, ionones, myrcene, L-menthol, N,N-diethylnerylamine, geraniol, nerolidols, flavours, fragrances, fuel additive, plastics, polyisoprene,

The method according to an embodiment further comprising converting the butadiene into a product selected from styrene-butadiene rubber, synthetic rubber, tires, component of tires, thermoplastic rubber, shoes, shoe soles, adhesives, sealants, asphalt, polymer modification components, nylon, ABS resins, chloroprene/neoprene rubber, nitrile rubber, plastics, acrylics, acrylonitrile-butadiene-styrene resins, and synthetic elastomers.

One embodiment is directed to a method for chemical recycling, the method comprising: a pyrolysis, gasification, and/or partial oxidation process; provided to a gas fermentation process; provided to a chemical product manufacturing process to produce a product comprising butadiene, isoprenoid, ethylene, polyethylene terephthalate (PET), or any combination thereof; provided to a synthetic rubber production process; provided to a tire manufacturing process; provided to a process of using tires; provided a process for the collecting and shredding of used tires; and provided back to the pyrolysis, gasification, and/or partial oxidation process.

One embodiment is directed to a method for chemical recycling, the method comprising: 1) a pyrolysis, gasification, and/or partial oxidation process; 2) provided to a gas fermentation process; 3) provided to a chemical product manufacturing process to produce a product comprising butadiene, isoprenoid, ethylene, polyethylene terephthalate (PET), or any combination thereof; 4) provided to a synthetic rubber production process; 5) provided to a tire manufacturing process; 6) provided to a process of using tires; 7) provided a process for the collecting and shredding of used tires; and 8) provided back to the pyrolysis, gasification, and/or partial oxidation process.

One embodiment is directed to a method for chemical recycling, the method comprising: 1) a pyrolysis, gasification, and/or partial oxidation process; 2) provided to a gas fermentation process; 3) provided to a chemical product manufacturing process to produce a commodity product; 4) provided to a synthetic rubber production process; 5) provided to a tire manufacturing process; 6) provided to a process of using tires; 7) provided a process for the collecting and shredding of used tires; and 8) provided back to the pyrolysis, gasification, and/or partial oxidation process.

Another embodiment is directed to a method for chemical recycling, the method comprising: 1) a pyrolysis, gasification, and/or partial oxidation process producing an effluent stream; 2) passing the effluent stream to a gas fermentation process to produce a product; 3) passing the gas fermentation product to a chemical product manufacturing process to produce a commodity product; 4) passing the commodity product to a synthetic rubber production process to produce synthetic rubber; 5) passing the synthetic rubber product to a tire manufacturing process to produce a tire; 6) providing the tire to a process of using tires; 7) passing the used tires to a process for the collecting and shredding of used tires; and 8) recycling used tires back to the pyrolysis, gasification, and/or partial oxidation process.

One embodiment is directed to provides a method and a genetically engineered microorganism capable of producing ethylene from a gaseous substrate, the microorganism comprising a heterologous nucleic acid encoding an ethylene-forming enzyme (EFE).

In some aspects of the method disclosed herein, the microorganism is a recombinant C1-fixing microorganism capable of producing ethylene from a gaseous substrate comprising a nucleic acid encoding a group of exogenous enzymes comprising ethylene-forming enzyme (EFE).

In some aspects of the microorganism disclosed herein, the microorganism is directed to a recombinant C1-fixing microorganism capable of switching cellular burden in production of ethylene, the microorganism comprising a nucleic acid encoding a group of exogenous enzymes comprising ethylene-forming enzyme (EFE) and one or more inducible promoters.

The microorganism of an embodiment, further comprising a nucleic acid encoding a group of exogenous enzymes comprising alpha-ketoglutarate permease (AKGP), wherein the nucleic acid is operably linked to a promoter.

The microorganism of an embodiment, wherein the microorganism is selected from the group consisting of Cupriavidus necator and Ralstonia eutropha.

The microorganism of an embodiment, wherein the microorganism is Cupriavidus necator.

The microorganism of an embodiment, further comprising a nucleic acid encoding alpha-ketoglutarate permease, wherein the nucleic acid is codon optimized for expression in the microorganism.

The microorganism of an embodiment, wherein the one or more inducible promoters is selected from an H₂ inducible promoter, a phosphate limited inducible promoter, a nitrogen limited inducible promoter, or any combination thereof.

The microorganism of an embodiment, wherein the inducible promoter is a phosphate limited inducible promoter.

The microorganism of an embodiment, wherein phosphate concentration is about 0-0.5 mM.

The microorganism of an embodiment, wherein phosphate concentration is about 0.52 mM.

The microorganism of an embodiment, wherein the EFE is codon optimized for expression in the microorganism.

The microorganism of an embodiment, further comprising a disruptive mutation in one or more genes.

The microorganism of an embodiment, wherein ethylene is converted into a derivative material selected from polyethylene (PE), polyethylene terephthalate (PET), polyvinyl chloride (PVC), ethylene vinyl acetate (EVA), sustainable aviation fuel (SAF), or any combination thereof.

The microorganism of an embodiment, wherein the gaseous substrate comprises CO₂ and an energy source.

The microorganism of an embodiment, wherein the gaseous substrate comprises CO₂, and H₂, O₂, or both.

One embodiment is directed to a method for the continuous production of ethylene, the process comprising: passing a gaseous substrate to a bioreactor containing a culture of a recombinant C1-fixing microorganism according to claim 1, in a culture medium such that the microorganism converts the gaseous substrate to ethylene; and recovering the ethylene from the bioreactor.

One embodiment is directed to a method of culturing the microorganism according to an embodiment, comprising growing the microorganism in a medium comprising a gaseous substrate, wherein the gaseous substrate comprises CO₂.

The method of an embodiment, wherein the gaseous substrate comprises an industrial waste product or off-gas.

The method of an embodiment, further comprising an energy source.

The method of an embodiment, wherein the energy source is provided intermittently.

The method of an embodiment, wherein the energy source is H₂.

One embodiment is a directed to a method comprising growing the microorganism in a medium comprising a gaseous substrate, wherein the gaseous substrate comprises CO₂ and an energy source.

The method of an embodiment further comprises co-producing ethylene and microbial biomass.

The method of an embodiment, wherein switching the cellular burden comprises a step of limiting the intracellular oxygen concentration.

The method of an embodiment, wherein switching the cellular burden comprises a step of limiting dissolved oxygen concentration.

The method of an embodiment, wherein the dissolved oxygen concentration is at least of about 0.5% saturation (% sat.) to 1.0% sat. and at most of about 50% sat. to 60% sat.

The method of an embodiment, wherein the dissolved oxygen concentration is at least of about 0.5% sat. to 1.0% sat. and at most of about 60% sat. to 70% sat.

The method of an embodiment, wherein the dissolved oxygen concentration is at least of about 0.5% sat. to 1.0% sat. and at most of about 70% sat. to 80% sat.

The method of an embodiment, wherein the dissolved oxygen concentration is at least of about 0.5% sat. to 1.0% sat. and at most of about 80% sat. to 90% sat.

The method of an embodiment, wherein the dissolved oxygen concentration is at least of about 0.5% sat. to 1.0% sat. and at most of about 90% sat. to 100% sat.

The method of an embodiment, wherein the dissolved oxygen concentration is at least of about 0.01% sat. to 1.0% sat. and at most of about 50% sat. to 60% sat.

The method of an embodiment, wherein the dissolved oxygen concentration is at least of about 0.01% sat. to 1.0% sat. and at most of about 60% sat. to 70% sat.

The method of an embodiment, wherein the dissolved oxygen concentration is at least of about 0.01% sat. to 1.0% sat. and at most of about 70% sat. to 80% sat.

The method of an embodiment, wherein the dissolved oxygen concentration is at least of about 0.01% sat. to 1.0% sat. and at most of about 80% sat. to 90% sat.

The method of an embodiment, wherein the dissolved oxygen concentration is at least of about 0.01% sat. to 1.0% sat. and at most of about 90% sat. to 100% sat.

The method of an embodiment, wherein O₂ is fed into an inlet from about 4 vol. % to about 30 vol. %.

The method of an embodiment, wherein the O₂ is fed into an inlet from about 1 vol. % to about 50 vol. %.

The method of an embodiment, wherein switching the cellular burden comprises a step of limiting steady state phosphate concentration.

The method of an embodiment, wherein switching the cellular burden comprises a step of limiting steady state phosphate concentration of about OmM to about 0.50 mM.

The method of an embodiment, wherein switching the cellular burden comprises a step of limiting steady state phosphate concentration of about 0.05 mM to about 0.50 mM.

The method of an embodiment, wherein switching the cellular burden comprises a step of limiting steady state phosphate concentration of about 0.01 mM to about 0.60 mM.

The method of an embodiment, wherein switching the cellular burden comprises a step of limiting steady state phosphate concentration of about 0.01 mM to about 0.70 mM.

The method of an embodiment, wherein switching the cellular burden comprises a step of limiting steady state phosphate concentration of about 0.01 mM to about 0.80 mM.

The method of an embodiment, wherein switching the cellular burden comprises a step of limiting steady state phosphate concentration of about 0.01 mM to about 0.90 mM.

The method of an embodiment, wherein switching the cellular burden comprises a step of limiting steady state phosphate concentration of about 0.01 mM to about 1.0 mM.

The method of an embodiment, wherein switching the cellular burden comprises a step of limiting steady state phosphate concentration of about 0.52 mM.

The method of an embodiment, wherein the microbial biomass is suitable as animal feed.

The method of an embodiment, wherein the gaseous substrate further comprises H₂, 02, or both.

In some aspects of the microorganism disclosed herein, the microorganism produces a commodity chemical product, microbial biomass, single cell protein (SCP), one or more intermediates, or any combination thereof.

In some aspects, the microbial biomass has a unit value. In one embodiment, the microbial biomass has a market value.

In some aspects of the microorganism disclosed herein, the microorganism is derived from a parental bacterium selected from the group consisting of Cupriavidus necator.

In some aspects of the microorganism disclosed herein, where the product is selected from the group 1-butanol, butyrate, butene, butadiene, methyl ethyl ketone, ethylene, acetone, isopropanol, lipids, 3-hydroxypropionate, terpenes, isoprene, fatty acids, fatty alcohols, 2-butanol, 1,2-propanediol, 1-propanol, 1-hexanol, 1-octanol, chorismate-derived products, 3-hydroxybutyrate, 1,3-butanediol, 2-hydroxyisobutyrate or 2-hydroxyisobutyric acid, isobutylene, adipic acid, keto-adipic acid, 1,3-hexanediol, 3-methyl-2-butanol, 2-buten-1-ol, isovalerate, isoamyl alcohol, or monoethylene glycol.

The disclosure further provides the genetically engineered C1-fixing microorganism, further comprising a microbial biomass and at least one excipient.

The disclosure further provides the genetically engineered C1-fixing microorganism, wherein the animal feed is suitable for feeding to one or more of beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents.

The disclosure further provides the genetically engineered C1-fixing microorganism, wherein the microorganism is suitable as a single cell protein (SCP).

The disclosure further provides the genetically engineered C1-fixing microorganism, wherein the microorganism is suitable as a cell-free protein synthesis (CFPS) platform.

The disclosure further provides the genetically engineered C1-fixing microorganism, wherein the product is native to the microorganism.

In some aspects of the method disclosed herein, the substrate comprises one or more of CO, CO₂, and H₂.

According to another embodiment, the claimed microorganism can be modified in order to directly produce a commodity chemical as described in U.S. Patent Application Publication No. 2023/0092645A1, the disclosure of which is incorporated by reference herein. In one embodiment, wherein the commodity chemical is selected from ethanol, isopropanol, monoethylene glycol, sulfuric acid, propylene, sodium hydroxide, sodium carbonate, ammonia, benzene, acetic acid, ethylene oxide, formaldehyde, methanol, or any combination thereof. In one embodiment, the commodity chemical is aluminum sulfate, ammonia, ammonium nitrate, ammonium sulfate, carbon black, chlorine, diammonium phosphate, monoammonium phosphate, hydrochloric acid, hydrogen fluoride, hydrogen peroxide, nitric acid, oxygen, phosphoric acid, sodium silicate, titanium dioxide, or any combination thereof. In another embodiment, the commodity chemical is acetic acid, acetone, acrylic acid, acrylonitrile, adipic acid, benzene, butadiene, butanol, caprolactam, cumene, cyclohexane, dioctyl phthalate, ethylene glycol, methanol, octanol, phenol, phthalic anhydride, polypropylene, polystyrene, polyvinyl chloride, polypropylene glycol, propylene oxide, styrene, terephthalic acid, toluene, toluene diisocyanate, urea, vinyl chloride, xylenes, or any combination thereof. The method according to one embodiment, wherein the commodity chemical is utilized in the sector selected from plastics, synthetic fibers, synthetic rubber, dyes, pigments, paints, coatings, fertilizers, agricultural chemicals, pesticides, cosmetics, soaps, cleaning agent, detergents, pharmaceuticals, mining, or any combination thereof.

In another embodiment, the method includes incorporating a commodity chemical into one or more articles or converting a commodity chemical into a product selected from ethanol, acetate, 1-butanol, butyrate, 2,3-butanediol, lactate, butene, butadiene, methyl ethyl ketone (2-butanone), ethylene, acetone, isopropanol, lipids, 3-hydroxyproprionate, terpenes, isoprene, fatty acids, 2-butanol, 1,2-propanediol, 1 propanol, 1 hexanol, 1 octanol, chorismate-derived products, 3-hydroxybutyrate, 1,3-butanediol, 2-hydroxyisobutyrate, 2-hydroxyisobutyric acid, isobutylene, adipic acid, 1,3-hexanediol, 3-methyl-2-butanol, 2-buten-1-ol, isovalerate, isoamyl alcohol, monoethylene glycol, antiseptic hand rubs, therapeutic treatments for methylene glycol poisoning, therapeutic treatments for methanol poisoning, pharmaceutical solvent for pain medication, oral hygiene products, antimicrobial preservative, engine fuel, rocket fuel, plastics, fuel cells, home fireplace fuels, industrial chemical precursor, cannabis solvent, winterization extraction solvent, paint masking product, paint, tincture, purification and extraction of DNA and RNA, cooling bath for various chemical reactions, ethylene to raw material, anaesthetic, ethylene and nitrogen in fruit ripening, fertilizer, safety glass, oxy-fuel in metal cutting, welding, high velocity thermal spraying, refrigerant, raw material to polyethylene, raw material to PET, raw material to PVC, fibers, packaging, coatings, adhesives, ethylene dichloride (EDC), vinyl chloride monomer (VCM), alpha olefins, linear alpha olefins, detergent alcohols, plasticizer alcohols, vinyl acetate monomer (VAM), barrier resins, industrial ethanol, ethyl acetate, ethyl acrylate, polyethylene oligomers, Ultra-high-molecular-weight polyethylene (UHMWPE), Ultra-low-molecular-weight polyethylene (ULMWPE or PE-WAX), High-molecular-weight polyethylene (HMWPE), High-density polyethylene (HDPE), High-density cross-linked polyethylene (HDXLPE), Cross-linked polyethylene (PEX or XLPE), Medium-density polyethylene (MDPE), Low-density polyethylene (LDPE), Very-low-density polyethylene (VLDPE), Chlorinated polyethylene (CPE), films, food packaging, non-food packaging, shrink film, stretch film, containers, drums, household goods, caps, pallets, pipes, refuse sacks, carrier bags, industrial lining, ethylene oxide, ethoxylates, shampoo, kitchen cleaners, glycol ethers, ethanolamines, surfactants, personal care products, polyester fibers, textiles, nonwovens, cover stock for diapers, road-building fabrics, filters, fiberfill, felts, transportation upholstery, paper reinforcement, tape reinforcement, tents, rope, cordage, sails, fish netting, seatbelts, laundry bags, synthetic artery replacements, carpets, rugs, apparel, sheets, pillowcases, towels, curtains, draperies, bed ticking, blankets, liquid coolant, antifreeze, preservative, dehydrating agent, drilling fluid, polyester resins, insulation materials, polyester film, de-icing fluid, heat transfer fluid, automotive antifreeze, water-based adhesives, latex paints, asphalt emulsions, electrolytic capacitors, synthetic leather, polyester resin PET, jars, bottles, plastic bottles, high-strength fibers, Dacron, durable-press blends, insulated clothing, furniture filling, pillow filling, artificial silk, carpet fiber, automobile tire yarns, conveyor belts, drive belts, reinforcement for fire and garden hoses, nonwoven fabrics for stabilizing drainage ditches, culverts, railroad beds, nonwovens for diaper topsheets, disposable medical garments, high-strength plastics, magnetic recording tape, photographic film, as feedstock, solvents for cosmetics, inks, medicinal tablets, disinfectants, sterilizers, skin creams, purification of vegetable oil and fats, purification of animal oil and fats, cleaning agent, drying agent, aerosol solvent, derivative ketones, isopropylamines, isopropyl esters, propylene, polypropylene oligomers, polymerization modifier, coupling agent, heat resistant articles, kettles, food containers, disposable bottles, clear bags, flooring, mats, adhesive stickers, foam polypropylene, building materials, hydrophilic clothing, medical dressings, or any combination thereof.

In another embodiment, the method includes incorporating a commodity chemical into an article or converting a commodity chemical into a product selected from humectants, filters, fire extinguishing sprinkler system, fuel for warming foods, heat transfer fluids, non-reacted component in formulation, deodorizing or air purifying, softening agent, arts/craft glue/paste, toys, children products, freezer gel pack, treating wood rot and fungus, preserving biological tissues and organs, alkyd type resins, resin esters, enamels, lacquers, latex paint, asphalt emulsion, thermoplastic resin, hydrate inhibition agent, agent for removing water vapor, shoe polish, vaccines, screen cleaning solution, water-based hydraulic fluid, heat transfer for liquid cooled computers, personal lubricant, lubricant, toothpaste, anti-foaming agent in food industrial applications, flame resistant hydraulic fluids, additive for electrolytic polishing belts, industrial solvent, trash bags, shower curtains, cups, utensils, medical devices, durable goods, nondurable goods, plastic sacks, plastic lids, industrial strapping, construction materials, felt, ovenable trays, frozen food trays, microwavable tray, artificial vascular scaffolds, vascular prostheses, woven devices, polyester-based prostheses, vehicle liner material, soaps, cosmetic products, laundry detergent jugs, laundry detergent, soap microplastic, microbeads, cosmetic product microbeads, detergent pods, disinfectant with scrubbing agents, toothpaste with microbeads, face wash, conditioner, body wash, hand cleaner, exfoliating products, bath products, shower gels, powder laundry detergent, lotions, deodorants, toilet cleaners, sunscreen, shopping bags, mouthwash bottles, peanut butter containers, salad dressing and vegetable oil containers, polar fleece fiber, tote bags, paneling, milk jugs, juice bottles, bleach bottles, motor oil bottles, cereal box liners, recycling containers, floor tile, drainage pipe, benches, picnic tables, fencing, wire jacketing, sliding windows, decks, mud flaps, roadway gutters, speed bumps, squeezable bottles, bread, dry cleaning bags, trash can liners, trash cans, compost bins, shipping envelopes, lumber, syrup bottles, ketchup bottles, straws, medicine bottles, battery cables, battery cases, disposable plates and cups, egg cartons, carry-out containers, compact disc cases, signs and displays, synthetic fibers, yarn, stable phase change material, thermal energy storage material, nylon 6,6, nylon, tires, rubber, adiponitrile, shoes, footwear, or any combination thereof.

### IV. PRODUCTS AND USES

The present disclosure provides edible products, food, animal feed, and others products, such as single cell protein (SCP) products or cultured protein, that include protein derived from the engineered C. necator bacterium provided herein. The present disclosure also provides alternative uses for the disclosed microorganisms including, but not limited to, incorporation into cosmetics or lotions, pharmaceuticals, and fertilizers. Additionally provided are methods of processing nutrients and other useful macromolecules using the engineered C. necator described herein.

A SCP product, cultured protein, “protein product,” or “microbial protein product” (e.g., one or more of single cell protein, cell lysate, protein concentrate, protein isolate, protein extract, protein hydrolysate, free amino acids, peptides, oligopeptides, or combinations thereof), derived from one or more engineered C. necator strains described herein, may be processed or incorporated into an edible food composition for human and/or animal consumption, a cosmetic, a pharmaceutical product, or a fertilizer.

A food composition (i.e., food product) may be, for example, a food item, a food ingredient, a nutritional product, an animal feed, and/or a pet food product. In some embodiments, the food composition may contain any of at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to 100% microbial protein product by weight, e.g., by weight on a dry weight basis.

A cosmetic or pharmaceutical composition may contain any of at least about 0.01%, at least about 0.05%, at least about 0.1%, at least about 0.5%, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to 100% microbial protein product by weight, e.g., by weight on a dry weight basis.

A fertilizer may contain any of at least about 0.5%, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to 100% microbial protein product by weight, e.g., by weight on a dry weight basis.

The biomass that is produced from culturing the engineered C. necator strains disclosed herein results in a desirable protein content. The biomass can have a protein content higher than or at least about any of about 40%, about 50%, about 60%, about 70%, about 80%, about 90%, or about 95% by weight, and a fat content of about 60%, about 50%, about 40%, about 30%, about 20%, about 15%, about 10%, or about 5% by weight.

Compositions comprising the disclosed SCP product can find application in various industries such as food, pharmaceutical, nutraceutical, cosmetic, agriculture, consumer goods, construction etc. The compositions can be used as food agents for human or animal consumption, cosmetic agents, pharmaceutical agents, nutritional agents, fertilizer and other agricultural agents, industrial agents, or any combination thereof. Illustrative applications include animal feed, food and beverages, infant formula, toddler formula, special dietary needs formula, reduced allergenicity formulas, skin-care and hair-care compositions, fertilizers, etc.

In some aspects, the SCP products as described herein are utilized in the production of a vegetarian or vegan food product. In certain embodiments, the SCP products are utilized in the production of an organic food product and/or pesticide-free and/or herbicide-free and/or fungicide-free and/or antibiotic-free food product. The SCP products may be utilized in a probiotic food product or in a prebiotic food product. In some aspects the SCP product may not include animal protein or fats.

In some embodiments, the SCP product can be incorporated into food products including, but not limited to, dairy products, dairy replacement products, meat products (including livestock, game, poultry, fish, or seafood products), meat replacement and/or imitation meat products (including imitation livestock, game, poultry, fish, or seafood products), bakery products, confections, health and protein bars, protein powders, sports and/or energy drinks, and/or protein shakes and/or smoothies. The type of food and beverage is not particularly limited, and can include, for example, noodles, instant noodles, soups, instant soups, pasta, microwave foods, canned foods, freeze-dried foods, soft drinks, fruit juice drinks, vegetable drinks, infant formula, toddler formula, non-dairy milk, coffee drinks, tea drinks, nutritional beverages, powdered beverages, protein powders, nutritional supplements, concentrated beverages, alcoholic beverages, breads, cake mixes, rice cakes, flour products, chewing gum, gummies, chocolate, caramel, cookies, snacks, chips, pretzels, crackers, biscuits, cakes, pies, confectionery, sauces, processed seasonings, flavor seasonings, cooking mixes, curries, stews, sauces, dressings, oils and fats, butter, margarine, mayonnaise and other condiments, milk drinks, yogurt, lactic acid bacteria drinks, ice creams, cream processed fish products, processed livestock products, agricultural canned products, jams and marmalades, pickles, cereals, nutritional foods, vegan or vegetarian meat substitutes, and the like. In certain embodiments, protein products are textured for incorporation into meat products and/or imitation meat products.

The SCP product may impart improved nutrition, water absorption, fat binding properties, texture, and/or eating qualities to a food product, such as a cereal based product. The SCP product may be used to fortify or is otherwise incorporated into a cereal based product. The cereal based product may be a breakfast cereal, cookie, cake, pie, brownie, muffin, or bread. In some aspects, the protein product may be used as a replacement for milk proteins (e.g. sodium caseinate) and/or as a vitamin and/or mineral supplement in milk or dairy products. The protein SCP ingredient may be used in one or more of non-fat dried milk, powdered milk, or dairy type drinks, such as, but not limited to, instant breakfast mixes, or imitation dairy type drinks including but not limited to soy milk, rice milk, and almond milk. The SCP product ingredient may be used in nutritionally fortified (e.g., protein, vitamin, and/or mineral fortified) candies, deserts, or treats.

The SCP product may be processed to produce a food product or ingredient thereof, in a process that includes heating the protein product, optionally in combination with other ingredients, optionally under shearing agitation, followed by extrusion to produce a product of desired texture (e.g., chewy, crunchy, crispy, resists dispersion in water, etc.). The SCP product can be processed to produce a food product or ingredient thereof, in a process that includes combining the SCP product with one or more additional protein sources (including, but not limited to, pea, rice, glutinous rice, wheat, gluten, soy, hemp, canola, insects, algae, and/or buckwheat).

In some aspects, free amino acids are included, either as part of the SCP product or supplemental to the SCP product, to impart a desired flavor. In one non-limiting embodiment, glutamic acid is included, thereby imparting an umami flavor to the food product.

In some aspects, for example, in a meat substitute or artificial meat product, a hydrogel, lipogel, and/or emulsion can be combined the SCP product, for example, as an agent release system (e.g., for release of a coloring agent, a flavor agent, a fatty acid, a leavening agent, a gelling agent (e.g., bicarbonate (e.g., potassium bicarbonate), calcium hydroxide, and/or alginate (e.g., sodium or potassium alginate)), wherein the agent(s) may be released during cooking of the food product to simulate animal meat).

In some aspects, a food product comprising the SCP produce may also include one or more plant protein source such as, but not limited to, pea, rice, glutinous rice, wheat, gluten, soy, hemp, canola, insects, algae, and/or buckwheat, in combination with a protein product produced by microorganisms as described herein (e.g., one or more of single cell protein, cell lysate, protein concentrate, protein isolate, protein extract, protein hydrolysate, free amino acids, peptides, oligopeptides, or combinations thereof), wherein the protein product imparts a flavor to the food composition, such as, for example, a meat-like flavor (including a livestock, game, poultry, or seafood meat-like flavor).

In some aspects, a food product, for example, a meat substitute or artificial meat product, includes a heme compound, such as a heme-containing polypeptide. In one embodiment, the food product includes heme (e.g., heme-containing polypeptide) from the microorganism from which the protein product is derived.

A meat substitute or artificial or imitation meat product (e.g., a livestock (e.g., beef, pork), game, poultry, fish, or seafood analogue product) may include a protein product produced by microorganisms as described herein (e.g., one or more of single cell protein, cell lysate, protein concentrate, protein isolate, protein extract, protein hydrolysate, free amino acids, peptides, oligopeptides, or combinations thereof). In some embodiments, the meat analogue product is a vegan product that does not contain any ingredients from animal sources. In some embodiments, an enhanced meat product which contains animal protein (e.g., a beef, poultry, pork, fish, seafood, or egg product) and comprises a protein product ingredient produced by microorganisms as described herein (e.g., one or more of single cell protein, cell lysate, protein concentrate, protein isolate, protein extract, protein hydrolysate, free amino acids, peptides, oligopeptides, or combinations thereof)), is provided. For example, the protein product may be included as an extender in an enhanced meat product or in a meat analogue product, e.g., the SCP product replaces any of at least about 10%, at least about 20%, at least about 30%, at least about 40%, at least about 50%, at least about 60%, or at least about 70% of the meat ingredient or an artificial or imitation meat ingredient (for example, a plant-based artificial or imitation meat analogue ingredient) to produce the enhanced meat product or meat analogue/imitation meat product, respectively.

In some aspects, the protein product is used as an aquaculture feed or in an aquaculture feed formulation. In some embodiments, the protein-rich biomass is used as a high-protein substitute for fishmeal used in aquaculture and/or other animal feed products. The animal feed may include up to 20% (w/w) or up to 10% (w/w) SCP product, wherein the SCP product comprises engineered C. necator cells described herein.

Protein and/or biomass produced according to the present disclosure may be converted to animal feed using methods and processes well known in the art and science of chemistry, chemical engineering, and food science. The feed produced through the disclosure may be used to grow organisms including but not limited to one or more of the following: other microorganisms, yeast, fungi, zooplankton, shellfish (e.g., shrimp, prawns, crabs, scallops, clams, mussels, etc.) or other invertebrates, fish, birds, and mammals. In certain non-limiting embodiments, the fish include but are not limited to one or more of: tilapia, tuna, salmon, cod, cobia, and haddock. The birds may include, but are not limited, to chickens, pheasants, or turkeys. The mammals may include but are not limited to one or more of: rodents, rabbits, goats, sheep, pigs, cows, horses, deer, dogs, cats, buffalo, llamas, alpacas, non-human primates, and aquatic mammals (e.g., dolphins, whales, manatees, etc.). The feed may be used to grow live-feed that in turn sustain finfish larvae through the first weeks of life. The feed produced may be used to grow zooplankton organisms including but not limited to one or more of the following: rotifers [Phylum Rotifera]; order Cladoceran (e.g., Daphnia sp., Moina sp.); sub-class Copepoda (e.g., Cyclops); Brine shrimp (Anemia sp.).

In some embodiments, the animal feed comprises up to 1% (w/w), up to 5% (w/w), up to 10% (w/w), up to 15% (w/w), up to 20% (w/w), up to 25% (w/w), up to 30% (w/w), up to 35% (w/w), up to 40% (w/w), up to 45% (w/w), up to 50% (w/w), up to 55% (w/w), up to 60% (w/w), up to 65% (w/w), up to 70% (w/w), up to 75% (w/w) or more of one or more of the disclosed SCP products. In some embodiments, the animal feed comprises at least 1% (w/w), at least 5% (w/w), at least 10% (w/w), at least 15% (w/w), at least 20% (w/w), at least 25% (w/w), at least 30% (w/w), at least 35% (w/w), at least 40% (w/w), at least 45% (w/w), at least 50% (w/w), at least 55% (w/w), at least 60% (w/w), at least 65% (w/w), at least 70% (w/w), at least 75% (w/w) or more of one or more of the disclosed SCP products. In some embodiments, the animal feed comprises about 1% (w/w), about 5% (w/w), about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w) or more of one or more of the disclosed SCP products.

The microbial cells of the present disclosure may be boiled prior to feeding to another organism (e.g., human or animal). The cells may sonicated, or otherwise lysed or ruptured prior to feeding to another organism (e.g., human or animal).

In addition to consumable or edible food products (e.g., human foods and animal feed), SCP products or biomass produced by or derived from the engineered C. necator disclosed herein can also be used as a fertilizer. The fertilizer may be applied to crop plants, ornamentals, turf grass, or aquacultures (e.g., algae or seaweed).

In some embodiments, the fertilizer comprises up to 1% (w/w), up to 5% (w/w), up to 10% (w/w), up to 15% (w/w), up to 20% (w/w), up to 25% (w/w), up to 30% (w/w), up to 35% (w/w), up to 40% (w/w), up to 45% (w/w), up to 50% (w/w), up to 55% (w/w), up to 60% (w/w), up to 65% (w/w), up to 70% (w/w), up to 75% (w/w) or more of one or more of the disclosed SCP products. In some embodiments, the fertilizer comprises at least 1% (w/w), at least 5% (w/w), at least 10% (w/w), at least 15% (w/w), at least 20% (w/w), at least 25% (w/w), at least 30% (w/w), at least 35% (w/w), at least 40% (w/w), at least 45% (w/w), at least 50% (w/w), at least 55% (w/w), at least 60% (w/w), at least 65% (w/w), at least 70% (w/w), at least 75% (w/w) or more of one or more of the disclosed SCP products. In some embodiments, the fertilizer comprises about 1% (w/w), about 5% (w/w), about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w) or more of one or more of the disclosed SCP products.

Starting with wet or dry microbial biomass produced as described herein, in certain embodiments, protein may be concentrated using a process comprising one or more of the following steps: liquid-solid extraction, removal and recovery of a solvent from the liquid extract, removal and recovery of a solvent from the solid (e.g., the protein concentrate), and drying and grinding of the solid (e.g., the protein concentrate).

A solid-liquid extraction may be performed batchwise or continuously. The solid-liquid extraction may be performed using one or more of: horizontal belt extractors; basket extractors; stationary extractors; and/or rotary cell extractors.

A non-polar solvent may be utilized in a solvent extraction step. A non-polar solvent may be utilized in combination with an alcohol solvent. A non-polar solvent may be utilized in combination with an aqueous alcohol solution. A non-polar solvent can be utilized to extract neutral lipids from an extract produced using alcohol and/or an aqueous alcohol solution. The non-polar solvent may be utilized that has a boiling point range (i.e., distillation range) of 65° C. to 70° C. A non-polar solvent may be utilized that consists primarily of six-carbon alkanes. In certain aspects, hexane is utilized as a non-polar solvent. The hexane utilized as a non-polar solvent may comply with the strict quality specifications required for the extraction of edible oils from soybean and other plant-based sources, including but not limited to: boiling (distillation) range, maximum non-volatile residue, flash point, maximum sulfur, maximum cyclic hydrocarbons, color and specific gravity. In certain aspects, “supercritical extraction” using liquid carbon dioxide under high pressure is utilized for solvent extraction.

The cell mass, i.e., microbial biomass produced as described herein may be kept in liquid suspension when subjected to solvent extraction or if dried, may be fed as a loose power with open, porous structure into a solvent extraction process. The rate of extraction can be increased by applying one or more of agitation and/or increasing the temperature. Higher temperature can result in higher solubility of the extractable material (e.g., lipid), and/or higher diffusion coefficients.

Water-free (absolute) low aliphatic alcohols, such as ethanol or isopropanol, are suitable solvents for lipids at high temperature, but the solubility of oils in these solvents decreases drastically as the temperature is lowered. In certain embodiments, lipid extraction takes place at high temperature one or more alcohol, including but not limited to ethanol, isopropanol, and/or methanol. In certain such embodiments, the lipid extract is cooled, and lipid saturation occurs. In certain such embodiments, the excess lipid separates as a distinct phase, which can be recovered by a solid-liquid separation process, such as, but not limited to, centrifugation. In certain such embodiments, the solvent, i.e., alcohol(s), is reheated and sent back for solvent extraction.

When a concentration gradient is used to transfer the extractable substance out of a solid, keeping the gradient high can facilitate the extraction process. In certain embodiments, the principle of counter-current multistage extraction is utilized to exploit this effect. In certain embodiments, the solvent extraction process is divided into a number of contact stages. In certain embodiments, each stage comprises the mixing of solid, e.g., microbial biomass and/or protein concentrate, and the solvent phases, and the separation of the two streams after extraction is achieved. In certain embodiments, in going from one stage to the next, the solids, e.g., microbial biomass and/or protein concentrate, and the solvent flow in opposite directions. Thus, microbial biomass and/or protein concentrate with the lowest extractable content (e.g., lipids) are contacted with the leanest solvent, resulting in higher extractable yield (e.g., lipid yield) and high driving force throughout the extractor.

The cell culture may be harvested in a logarithmic phase and/or in an arithmetic phase and/or in a stationary phase. Extraction can be performed using batch, semi-continuous and/or continuous solvent extractors.

In batch processes, a certain quantity of microbial biomass and/or biological material is contacted with a certain volume of fresh solvent. In certain embodiments, the extract is drained off, distilled and the solvent is recirculated through the extractor until the residual extractable content (e.g., lipid content) in the batch of microbial biomass and/or biological material is reduced to a targeted level.

A semi-continuous solvent extraction system may utilize that consists of several batch extractors connected in series. In certain such embodiments, the solvent and/or extract flows from one extractor to the next one in the series. In certain non-limiting embodiments, a French Stationary Basket Extractor is utilized.

A continuous solvent extraction process may be utilized in which microbial biomass and/or biological material and/or protein concentrate and solvent are fed continuously into an extractor.

A protein product (e.g., one or more of single cell protein, cell lysate, protein concentrate, protein isolate, protein extract, protein hydrolysate, free amino acids, peptides, oligopeptides, or combinations thereof), is derived from and/or includes biomass and/or protein isolate, protein extract, protein hydrolysate, free amino acids, peptides, and/or oligopeptides derived from one or more engineered C. necator strains described herein and may be produced by any method described herein.

### V. EXAMPLES OF EMBODIMENTS

Embodiment 1. A non-naturally occurring C1-fixing strain capable of continuously growing autotrophically at up to about 40° C.

Embodiment 2. The strain according to embodiment 1, wherein the strain is selected from the group consisting of Cupriavidus necator and Ralstonia eutropha.

Embodiment 3. The strain according to any of embodiment 1 to 2, wherein the strain does not natively produce polyhydroxyalkanoates (PHAs).

Embodiment 4. The strain according to any of embodiments 1 to 3, wherein the strain continuously produces single-cell protein (SCP) from a gaseous substrate.

Embodiment 5. The strain according to any of embodiments 1 to 4, wherein gaseous substrate comprises CO2 and an energy source.

Embodiment 6. The strain according to any of embodiments 1 to 5, wherein the gaseous substrate comprises CO2, and H2, O2, or both.

Embodiment 7. The strain according to any of embodiments 1 to 6, further comprises the SCP converted to a nutritive ingredient.

Embodiment 8. The strain according to any of embodiments 1 to 7, further comprises genetic engineering to produce enhanced nutritive products.

Embodiment 9. The strain according to any of embodiments 1 to 8, wherein the products are selected from a yogurt, a smoothie, a bread product, a pasta product, a nutritional bar, a chip or cracker, a plant-based meat substitute, a cheese, a plant-based cheese, a powdered nutritional supplement, a dairy product, a dairy replacement product, a meat product, a bakery product, a confection, a protein bar, a protein powder, a sport and/or energy drink, a protein shake and/or smoothie, noodles, instant noodles, a soup, an instant soup, a microwaveable food, a canned food, a freeze-dried food, a soft drink, a fruit juice drink, a vegetable drink, an infant formula, a toddler formula, a non-dairy milk, a coffee drink, a tea drink, a nutritional beverage, a powdered beverage, a nutritional supplement, a concentrated beverage, an alcoholic beverage, a cake mix, a rice cake, a flour product, chewing gum, gummies, chocolate, caramel, a cookie, chips, pretzels, crackers, biscuits, cakes, pies, a sauce, a processed seasoning, a flavor seasoning, a cooking mix, a curry, a stew, a dressing, an oils/fat, a butter, a margarine, a mayonnaise and other condiments, a lactic acid bacteria drink, an ice cream, a cream processed fish product, a processed livestock product, an agricultural canned product, a jam or marmalade, a pickled product, and a cereal or cereal product.

Embodiment 10. The strain according to any of embodiments 1 to 9, wherein the strain is Cupriavidus necator DSM 34774.

Embodiment 11. The strain according to any of embodiments 1 to 10, wherein the strain has at least 95% (e.g., 95%, 96%, 97%, 98%, 99%, 100%, or any value therebetween) identity to Cupriavidus necator DSM 34774 or SEQ ID NOs: 1-3.

Embodiment 12. A method for the continuous production of single-cell protein (SCP), the process comprising: passing a gaseous substrate to a bioreactor containing a culture of a non-naturally occurring C1-fixing strain according to claim 1, in a culture medium such that the strain converts the gaseous substrate to SCP under autotrophic conditions; and recovering the SCP from the bioreactor.

Embodiment 13. The method according to embodiment 12, wherein the gaseous substrate comprises an industrial waste product or off-gas.

Embodiment 14. The method according to any of embodiments 12 to 13, further comprising an energy source.

Embodiment 15. The method according to any of embodiments 12 to 14, wherein the energy source is provided intermittently.

Embodiment 16. The method according to any of embodiments 12 to 15, wherein the gaseous substrate comprises CO2 and an energy source.

Embodiment 17. The method according to any of embodiments 12 to 16, wherein the energy source is H2.

Embodiment 18. The method according to any of embodiments 12 to 17, wherein the gaseous substrate further comprises H2, O2, or both.

Embodiment 19. The method according to any of embodiments 12 to 18, further comprising a step of limiting dissolved oxygen concentration, thereby switching a cellular burden.

Embodiment 20. The method according to any of embodiments 12 to 19, further comprising controlling iron concentrations.

Embodiment 21. A method for the continuous production of a food or feed ingredient, the process comprising: passing a gaseous substrate to a bioreactor containing a culture of a non-naturally occurring C1-fixing strain according to claim 1, in a culture medium such that the strain converts the gaseous substrate to SCP under autotrophic conditions; recovering the SCP from the bioreactor; and utilizing the SCP as an ingredient in food or feed.

Embodiment 22. A method for obtaining a nutritive product from a circular process, wherein the method comprises:
a) generating electric energy using a renewable and/or non-renewable source,
b) electrolyzing water or CO2 to produce at least H2, O2 or CO;
c) passing at least one of the H2, O2 or CO to a bioreactor containing a culture comprising a liquid nutrient medium and a non-naturally occurring C1-fixing strain according to claim 1; and
d) fermenting the culture to produce at least the nutritive product.

Embodiment 23. The method according to embodiment 22, wherein the electrolyzing has a cost per unit electric energy.

Embodiment 24. The method according to embodiment 22 or 23, further comprising passing at least a portion of the O₂ produced in the electrolysis process to a combustion or gasification process to produce the carbon dioxide.

Embodiment 25. The method according to any of embodiments 22 to 26, wherein electrolyzing is operated to supplement a C1 feedstock during time periods when the cost per unit electric energy is less than the cost per unit of C1 feedstock.

Embodiment 26. The method according to any of embodiments 22 to 25, wherein the renewable energy source comprises solar energy, wind power, wave power, tidal power, hydro power, geothermal energy, biomass and/or biofuel combustion, nuclear, or any combination thereof.

Embodiment 27. The method according to any of embodiments 22 to 26, wherein one or more of steps a), b), or c) are intermittent.

Embodiment 28. The method according to any of embodiments 22 to 27, wherein the strain is Cupriavidus necator DSM 34774 or a derivative thereof.

Embodiment 29. A system for producing a nutritive product comprising:
a) an electrolysis process in fluid communication with a renewable and/or non-renewable energy source for producing at least one of H₂, O₂, or CO;
b) an industrial plant for producing at least C1 feedstock; and
c) a bioreactor, in fluid communication with the electrolysis process and/or in continuous fluid communication with the industrial plant, comprising a reaction vessel suitable for growing, fermenting, and/or culturing and housing the non-naturally occurring C1-fixing strain of claim 1 to produce at least the nutritive product.

Embodiment 30. The system according to embodiment 29, further comprising at least one oxygen enriched combustion or gasification unit in fluid communication with the electrolysis process, the bioreactor, or both, the oxygen enriched combustion or gasification unit for producing carbon dioxide.

Embodiment 31. The system according to embodiment 29 or 30, further comprising at least one downstream processing system in fluid communication with the bioreactor selected from a recovery system, a purification system, an enriching system, a storage system, a recycling or further processing system for fermentation off-gas, hydrogen, water, oxygen, carbon dioxide, used medium and medium components, or combinations thereof.

Embodiment 32. The system according to any of embodiments 29 to 31, further comprising a cell processing unit, in fluid communication with the bioreactor, wherein the non-naturally occurring C1-fixing strain is further processed to a single cell protein (SCP) and/or a cell-free protein synthesis platform.

Embodiment 33. The system according to any of embodiments of 29 to 32, wherein the renewable energy source is selected from solar energy, wind power, wave power, tidal power, hydro power, geothermal energy, biomass and/or biofuel combustion, nuclear, or any combination thereof.

Embodiment 34. A two-stage fermentation method for producing a nutritive product, wherein the method comprises:
a) culturing a first microorganism under conditions wherein the first microorganism ferments a first feedstock to produce an intermediate; and
b) culturing a second microorganism under conditions wherein the second microorganism ferments the intermediate to produce the nutritive product from the intermediate; and wherein at least one of the first microorganism or the second microorganism is selected from a non-naturally occurring C1-fixing strain according to claim 1.

Embodiment 35. The method according to embodiment 34, wherein the non-naturally occurring C1-fixing strain is Cupriavidus necator DSM 34774.

Embodiment 36. The method according to embodiment 35 or 35, further comprising a methanogen.

Embodiment 37. A method for producing a protein-based bioplastic, wherein the method comprises:
a) culturing a non-naturally occurring C1-fixing strain according to claim 1 in a nutrient medium in the presence of a gaseous substrate to produce single-cell protein (SCP); and
b) processing the SCP to produce a protein-based bioplastic.

Embodiment 38. The method according to embodiment 37, wherein the processing step comprises one or more of sterilizing the SCP, centrifuging the SCP, drying the SCP, denaturing the SCP, and extracting the SCP.

Embodiment 39. The method according to embodiment 37 or 38, wherein the processing step comprises blending the SCP with a plasticizer.

Embodiment 40. The method according to any of embodiments 39 to 41, wherein the plasticizer is one or more of water, glycerol, ethylene glycerol, propylene glycerol, palmitic acid, diethyl tartarate, dibutyl tartarate, 1,2-butanediol, 1,3-butanediol, polyethylene glycol (PEG), sorbitol, mantitoletc, dimethylaniline, diphenylamine, and 2,3-butanediol.

Embodiment 41. The method according to any of embodiments 37 to 40, wherein the strain is Cupriavidus necator DSM 34774 or a derivative thereof.

Embodiment 42. One embodiment is directed to recombinant C1-fixing microorganism comprising a disruptive mutation at a membrane bound hydrogenase gene locus.

Embodiment 43. The microorganism according to embodiment 42, wherein the disruptive mutation is a deletion.

Embodiment 44. The microorganism according to any of embodiments 42 to 43, wherein the membrane bound hydrogenase is hoxKGZ.

Embodiment 45. The microorganism according to any of embodiments 42 to 44, wherein the microorganism is selected from the group consisting of Cupriavidus necator and Ralstonia eutropha.

Embodiment 46. The microorganism according to any of embodiments 42 to 45, wherein the strain does not natively produce polyhydroxyalkanoates (PHAs).

Embodiment 47. The microorganism according to any of embodiments 42 to 46, wherein the Cupriavidus necator is selected from the group consisting of DSM 428, DSM 531, DSM 541, and DSM 34774.

Embodiment 48. The microorganism according to any of embodiments 42 to 47, wherein the microorganism is Cupriavidus necator DSM 34774 or a derivative thereof.

Embodiment 49. The microorganism according to any of embodiments 42 to 48, wherein the microorganism has at least 95% (e.g., 95%, 96%, 97%, 98%, 99%, 100%, or any value therebetween) sequence identity to SEQ ID NO: 1-3.

Embodiment 50. One method of an embodiment is directed to a method for the continuous production of a product, the method comprising culturing the microorganism having a disruptive mutation at a membrane bound hydrogenase in the presence of a gaseous substrate.

Embodiment 51. The method according to embodiment 50, wherein the gaseous substrate comprises an industrial waste product or off-gas.

Embodiment 52. The method according to any of embodiments 50 to 51, further comprising an energy source.

Embodiment 53. The method according to any of embodiments 50 to 52, wherein the energy source is provided intermittently.

Embodiment 54. The method according to any of embodiments 50 to 53, wherein the gaseous substrate comprises CO₂ and an energy source.

Embodiment 55. The method according to any of embodiments 50 to 54, wherein the energy source is H₂.

Embodiment 56. The method according to any of embodiments 50 to 55, wherein the gaseous substrate further comprises H₂, O₂, or both.

Embodiment 57. The method according to any of embodiments 50 to 56, wherein the microorganism comprises a reduced H₂:CO₂ uptake ratio by minimizing H₂ oxidation to water.

Embodiment 58. The method according to any of embodiments 50 to 57, wherein the microorganism comprises an increase in the intracellular oxygen availability.

Embodiment 59. An embodiment is directed to a method for obtaining a product from a circular process, wherein the method comprises: generating electric energy using a renewable and/or non-renewable source, electrolyzing water or CO₂ to produce at least H2, O₂ or CO; passing at least one of the H₂, O₂ or CO to a bioreactor containing a culture comprising a liquid nutrient medium and a recombinant C1-fixing microorganism according to embodiment 1; and fermenting the culture to produce at least the product.

Embodiment 60. The method according to embodiment 59, wherein disruptive mutation is a deletion and the membrane bound hydrogenase is hoxKGZ.

Embodiment 61. The method according to any of embodiments 59 to 60, wherein the C1-fixing microorganism is selected from the group consisting of DSM 428, DSM 531, DSM 541, and DSM 34774.

Embodiment 62. A nutritive composition comprising a non-naturally occurring C1-fixing strain capable of continuously growing autotrophically at up to about 40° C.

Embodiment 63. The composition according to embodiment 62, further comprising a product selected from a yogurt, a smoothie, a bread product, a pasta product, a nutritional bar, a chip or cracker, a plant-based meat substitute, a cheese, a plant-based cheese, a powdered nutritional supplement, a dairy product, a dairy replacement product, a meat product, a bakery product, a confection, a protein bar, a protein powder, a sport and/or energy drink, a protein shake and/or smoothie, noodles, instant noodles, a soup, an instant soup, a microwaveable food, a canned food, a freeze-dried food, a soft drink, a fruit juice drink, a vegetable drink, an infant formula, a toddler formula, a non-dairy milk, a coffee drink, a tea drink, a nutritional beverage, a powdered beverage, a nutritional supplement, a concentrated beverage, an alcoholic beverage, a cake mix, a rice cake, a flour product, chewing gum, gummies, chocolate, caramel, a cookie, chips, pretzels, crackers, biscuits, cakes, pies, a sauce, a processed seasoning, a flavor seasoning, a cooking mix, a curry, a stew, a dressing, an oils/fat, a butter, a margarine, a mayonnaise and other condiments, a lactic acid bacteria drink, an ice cream, a cream processed fish product, a processed livestock product, an agricultural canned product, a jam or marmalade, a pickled product, and a cereal or cereal product.

Embodiment 64. The composition according to any of embodiments 62 to 63, further comprising the composition incorporated into one or more articles, converted into one or more second products, end-user products, consumer products, or any combination thereof.

Embodiment 65. The composition according to any of embodiments 62 to 64, wherein the composition is incorporated into pet food or animal feed.

### VI. EXAMPLES

The following examples further illustrate the disclosure but, of course, should not be construed to limit its scope in any way.

### Example 1: Generating a Scarless Deletion at the hoxKGZ Locus in Cupriavidus necator

### Homology Arm Amplification and Assembly

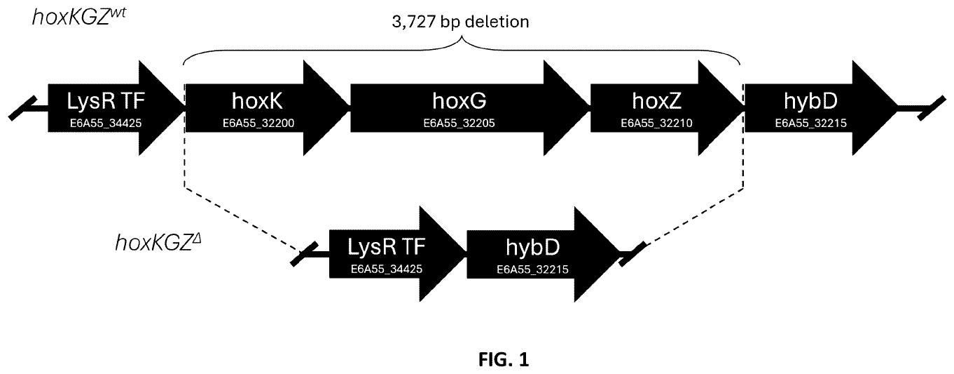

To generate a scarless deletion of the membrane bound dehydrogenase locus operon hoxKGZ in Cupriavidus necator (FIG. 1), 1000 bp homology arms were PCR amplified from C. necator H16 gDNA using Kappa 2× Master Mix and Gibson Assembled into the suicide plasmid pK18mobsacB. Briefly, the forward and reverse primers were designed to include 5′ and 3′ Gibson tails compatible with pK18mobsacB that was digested open with the restriction enzyme BamHI. The primer and homology arm sequences are provided in Table 1-1 below. Correct homology arm amplicon sizes were verified on a 1% agarose gel and column purified. Purified homology arms were combined at a mass of 100 ng each with 100 ng of BamHI-digested pK18mobsacB backbone in a 20 uL Gibson Assembly reaction and incubated at 50° C. for 60 mins. Next, the reaction mixture was diluted 1:5, and 3 μL of the diluted mixture was used to transform chemically competent DH5alpha Escherichia coli and plated on LB agar medium containing 50 ng/uL kanamycin antibiotic. Plates were incubated for 24 h at 37° C., and colonies were screened by PCR to verify the presence of each homology arm insert. Positive colonies were grown overnight in 5 mL of LB broth supplemented with 50 ng/uL kanamycin, mini-prepped, and plasmids were sequence verified.

### Transformation via Conjugation

The sequence verified pK18mobsacB plasmid containing left and right 1000 bp homology arms was electroporated into S17-1 E. coli cells and plated on LB supplemented with 50 ng/uL kanamycin. A single colony was picked and used to inoculate 5 mL of LB broth supplemented with 50 ng/uL kanamycin to generate the conjugation donor strain. To generate the conjugation recipient strain, a single colony of C. necator grown on LB agar supplemented with 10 ng/uL gentamycin was picked and used to inoculate 5 mL of LB broth supplemented with 10 ng/uL gentamycin. The donor E. coli culture was grown overnight at 37° C. with shaking at 250 rpm, while the recipient C. necator culture was grown overnight at 30° C. with shaking at 200 rpm. The following morning, cells were collected by centrifugation for 3 min at 6000 rpm at 25° C., and pellets were resuspended in 50 μL of LB medium. The 50 μL of donor cells and recipient cells were mixed and spotted on a sterile hydrophilic filter on LB agar without antibiotics.

### Selection and Counter-Selection

Plates were incubated overnight at 30° C., and cells were removed from the filter the following morning by folding it in half using sterile forceps and transferring to a tube containing 1 mL of LB. The cells were dislodged from the filter by vortexing, and a serial dilution was prepared (10⁰ to 10⁻³) and plated at 100 uL volumes on LB agar containing 200 ng/uL kanamycin and 10 ng/uL gentamycin. Plates were incubated for 3 days at 30° C. Next, colonies were replica-patched on LB agar plates containing 200 ng/uL kanamycin with 10 ng/uL gentamycin and LB agar plates containing 20% sucrose plus 200 ng/uL kanamycin with 10 ng/uL gentamycin. Colonies that grew on kanamycin but not sucrose plus kanamycin (primary integrants) were streak purified on LB agar plates containing 200 ng/uL kanamycin and 10 ng/uL gentamycin and subsequently cultured overnight at 30° C. in LB broth with only 10 ng/uL gentamycin. The following morning, 100 uL volumes of serial diluted (10⁰ to 10⁻³) culture was plated overnight at 30° C. on agar containing 20% sucrose and 10 ng/uL gentamycin to select for secondary recombinants. Sucrose-resistant colonies were patched on LB agar containing 20% sucrose and 10 ng/uL gentamycin (with and without 200 ng/uL kanamycin) to select for recombinants that were sucrose-resistant and kanamycin-sensitive. Finally, 24 Suc^{R} Kans colonies were prepped for colony PCR and sequencing to verify deletion of the hoxKGZ locus.

TABLE 1-1
Primer, Homology Arm, and Plasmid Sequences for hoxKGZ locus

| Sequence Type | Sequence |
|---|---|
| F-Primer Left | 5′-TCGAGCTCGGTACCCGGGGAACTACGCCACGAAACCTTC-3′ |
| Homology Arm | |
| R-Primer Left | 5′-TACGCTCTGAACCTGTCTCCTAATTTCTGT-3′ |
| Homology Arm | |
| F-Primer Right | 5′-AGACAGGTTCAGAGCGTAGTGAGCACGAT-3′ |
| Homology Arm | |
| F-Primer Right | 5′-TGCAGGTCGACTCTAGAGCGTTCGGCTTCGTAGCGATC-3′ |
| Homology Arm | |
| Left Homology | 5′- |
| Arm (500 bp) | TCGAGCTCGGTACCCGGGGAACTACGCCACGAAACCTTCCTGCTGCGCGAGCCGCATTCTG |
| | GCACTCGCTCGGTGGCCGAGGAGATGTTCGGGCACCACCTCTTTGCACCGGCCAAAGTGCC |
| | CATGCTGGATAGCAATGAAACGATCAAGTAAGCGGTGATGGCCGGCATGGGCATCAGCCT |
| | GTTATCACTGCACACGCTGTCGCTGGAATTGCACACACCGGGAGATCGCGCTGCTCGATGC |
| | GGCAGGCACGCCAATTGAGCGTGTCTGGCATGTCGTGCACATGGCTAGCAAGCGGCTGTC |
| | GCCCGCCAGCGAGCTATGTCGCACCTTCCTGCTGGAACACACGGCGGAATATTTGGAACG |
| | GGAATACGGCGGGCTGCTGCCCGGCGGGCTTGTGGCCTAGGGGATTCGGCAAGTCGGGGA |
| | TCCTGGTAGATGGCGTCGGCCTGCGCATGTGTCATGGCGCCGGTGGCGAGATAAGCATCCG |
| | GCACCACGGCAGTTGGGCTTGCGGGCTGGTCTGCAGTAGCTGCGTGGCATTGCTCGGACGA |
| | CAGACCCGCTCCTGCCGCGCATGCAGCAGGGGTACAGGAAGGCTGCGAGCAGCGCCGGCT |
| | CATTGCCTTTCCGTTGGGCGGGGCGAGACGCCGGGGGCGGGGGCTCATCCGAGTTCAACG |
| | CCGATCACTGAACTTCCTTCTGATGCATTCAAGCGAAAACCCAGTGAGCATCTGGCGTCGG |
| | CTAGCGCCAGGCGACGGTCCACTTCATGACGGATGAAATATTGTCAAATCAGGATCCGGT |
| | GTCCTGCGTTGTAGGTTCGCCGAATAGGGCGCTGTCGGGCGGACGCACGAACCTGCGTCAC |
| | AGATGCTCATACATGCCTTCTCGGTATCAATCTTTTTCTAAACAAGCCATCCAACTCAGGAT |
| | GGTAGCGGGGGTTTTCCCCAGGTCTTCGGATTCAGGCATAGATCTTGTTTCAACTATGTCG |
| | CCAAGCCAGCATTCGTGCGCGAGGGCGGTATCGCTCCCCGGTTGGCGCATCGCGACGAAT |
| | GCCAATACCAATACAGAAATTAGGAGACAGGTTCAGAGCGTA-3′ |
| Right Homology | 5′- |
| Arm (500 bp) | AGACAGGTTCAGAGCGTAGTGAGCACGATGGTCTCGGGCTATCGGACCTTTAAGGACTGA |
| | TGCGCAATGCAACCGAAGCGATCGCAGGCTTTCGTCGATACCATGCACCCGCTACCGGAG |
| | GTATTGCCCTCGGTATGGTGGCATCCCGGCGTGTCACGGCGCGTGTCGGTTCATTCGGGCT |
| | GTTTTCTGAGTAGTCATTACCTCAAGCCGCTCGGCCTGTCGCAGCCCCACGCCCGCTGTCTG |
| | CTCAGGATTGCGAAACGGCGCTTGCATGAAATCATCAATTGGAACAGCCGTCAGGCTTGG |
| | AAAGGCGCCTGTTCCTCCTTTCGTGTCTCGGTCGCTCGCCGCACGGTACACGGTTCCCTTTT |
| | TCGCTGACCACCGCCATGAGACCAACTCAAACGTCCGATATCGCACCTGGAACCAGCTGTA |
| | ACCAATCCCGTGACAAGGAGCCGGTGGTAGTTGCAATGGGCATTGGCAATGTGCTCTGGG |
| | CGGATGAGGGCTTTGGCGTGCGCTGCATTGAAACCCTCCAGCAGCGCTATCAGTTCGCACC |
| | GCAGGTATGCCTGGTCGACGGGGGCACGCAGGGGCTCTATCTGATCCACCACGTGCAGGC |
| | TGCGTCGCGCCTGCTGATCTTCGATGCGATCGACTATGGCCTGCCTCCAGGAACGCTGAGA |
| | ATTATCGAAGACGAGGCGGTGCCGAAATTCCTTGGGGCCAAGAAGATGAGCCTGCACCAG |
| | ACCGGCTTCCAGGAAGTACTGTTGCTCGCCCAGTTGACGGGACAGTACCCGCAGCAGGTG |
| | GTGCTGATCGGCTGTCAGCCGGAGGAGTTGGAGGATTATGGCGGCAGTCTAAGGCGGGTG |
| | ATGAAGGCTGCGGTGGAGGATGCGGTGGAGAAAGGGGCGGACTTGCTGCGGAGATGGGG |
| | CGGGATGCCGGTGCCTCGCACTGCGGAGCTTGCGCCTGCCGAGGCCGTGACCGTGCCGCAT |
| | CTGGCGCTCGATCGCTACGAAGCCGAACGCTCTAGAGTCGACCTGCA-3′ |
| pK18mobsacB | 5′-TGCCGCAAGCACTCAGGGCGCAAGGGCTGCTAAAGGAAGCGGAAC |
| empty suicide | ACGTAGAAAGCCAGTCCGCAGAAACGGTGCTGACCCCGGATGAATGTC |
| plasmid | AGCTACTGGGCTATCTGGACAAGGGAAAACGCAAGCGCAAAGAGAAAG |
| | CAGGTAGCTTGCAGTGGGCTTACATGGCGATAGCTAGACTGGGCGGTT |
| | TTATGGACAGCAAGCGAACCGGAATTGCCAGCTGGGGCGCCCTCTGGT |
| | AAGGTTGGGAAGCCCTGCAAAGTAAACTGGATGGCTTTCTTGCCGCCA |
| | AGGATCTGATGGCGCAGGGGATCAAGATCTGATCAAGAGACAGGATGA |
| | GGATCGTTTCGCATGATTGAACAAGATGGATTGCACGCAGGTTCTCCG |
| | GCCGCTTGGGTGGAGAGGCTATTCGGCTATGACTGGGCACAACAGACA |
| | ATCGGCTGCTCTGATGCCGCCGTGTTCCGGCTGTCAGCGCAGGGGCGC |
| | CCGGTTCTTTTTGTCAAGACCGACCTGTCCGGTGCCCTGAATGAACTC |
| | CAAGACGAGGCAGCGCGGCTATCGTGGCTGGCCACGACGGGCGTTCCT |
| | TGCGCAGCTGTGCTCGACGTTGTCACTGAAGCGGGAAGGGACTGGCTG |
| | CTATTGGGCGAAGTGCCGGGGCAGGATCTCCTGTCATCTCACCTTGCT |
| | CCTGCCGAGAAAGTATCCATCATGGCTGATGCAATGCGGCGGCTGCAT |
| | ACGCTTGATCCGGCTACCTGCCCATTCGACCACCAAGCGAAACATCGC |
| | ATCGAGCGAGCACGTACTCGGATGGAAGCCGGTCTTGTCGATCAGGAT |
| | GATCTGGACGAAGAGCATCAGGGGCTCGCGCCAGCCGAACTGTTCGCC |
| | AGGCTCAAGGCGCGGATGCCCGACGGCGAGGATCTCGTCGTGACCCAT |
| | GGCGATGCCTGCTTGCCGAATATCATGGTGGAAAATGGCCGCTTTTCT |
| | GGATTCATCGACTGTGGCCGGCTGGGTGTGGCGGACCGCTATCAGGAC |
| | ATAGCGTTGGCTACCCGTGATATTGCTGAAGAGCTTGGCGGCGAATGG |
| | GCTGACCGCTTCCTCGTGCTTTACGGTATCGCCGCTCCCGATTCGCAG |
| | CGCATCGCCTTCTATCGCCTTCTTGACGAGTTCTTCTGAGCGGGACTC |
| | TGGGGTTCGCTAGAGGATCGATCCTTTTTAACCCATCACATATACCTG |
| | CCGTTCACTATTATTTAGTGAAATGAGATATTATGATATTTTCTGAAT |
| | TGTGATTAAAAAGGCAACTTTATGCCCATGCAACAGAAACTATAAAAA |
| | ATACAGAGAATGAAAAGAAACAGATAGATTTTTTAGTTCTTTAGGCCC |
| | GTAGTCTGCAAATCCTTTTATGATTTTCTATCAAACAAAAGAGGAAAA |
| | TAGACCAGTTGCAATCCAAACGAGAGTCTAATAGAATGAGGTCGAAAA |
| | GTAAATCGCGCGGGTTTGTTACTGATAAAGCAGGCAAGACCTAAAATG |
| | TGTAAAGGGCAAAGTGTATACTTTGGCGTCACCCCTTACATATTTTAG |
| | GTCTTTTTTTATTGTGCGTAACTAACTTGCCATCTTCAAACAGGAGGG |
| | CTGGAAGAAGCAGACCGCTAACACAGTACATAAAAAAGGAGACATGAA |
| | CGATGAACATCAAAAAGTTTGCAAAACAAGCAACAGTATTAACCTTTA |
| | CTACCGCACTGCTGGCAGGAGGCGCAACTCAAGCGTTTGCGAAAGAAA |
| | CGAACCAAAAGCCATATAAGGAAACATACGGCATTTCCCATATTACAC |
| | GCCATGATATGCTGCAAATCCCTGAACAGCAAAAAAATGAAAAATATC |
| | AAGTTTCTGAATTTGATTCGTCCACAATTAAAAATATCTCTTCTGCAA |
| | AAGGCCTGGACGTTTGGGACAGCTGGCCATTACAAAACGCTGACGGCA |
| | CTGTCGCAAACTATCACGGCTACCACATCGTCTTTGCATTAGCCGGAG |
| | ATCCTAAAAATGCGGATGACACATCGATTTACATGTTCTATCAAAAAG |
| | TCGGCGAAACTTCTATTGACAGCTGGAAAAACGCTGGCCGCGTCTTTA |
| | AAGACAGCGACAAATTCGATGCAAATGATTCTATCCTAAAAGACCAAA |
| | CACAAGAATGGTCAGGTTCAGCCACATTTACATCTGACGGAAAAATCC |
| | GTTTATTCTACACTGATTTCTCCGGTAAACATTACGGCAAACAAACAC |
| | TGACAACTGCACAAGTTAACGTATCAGCATCAGACAGCTCTTTGAACA |
| | TCAACGGTGTAGAGGATTATAAATCAATCTTTGACGGTGACGGAAAAA |
| | CGTATCAAAATGTACAGCAGTTCATCGATGAAGGCAACTACAGCTCAG |
| | GCGACAACCATACGCTGAGAGATCCTCACTACGTAGAAGATAAAGGCC |
| | ACAAATACTTAGTATTTGAAGCAAACACTGGAACTGAAGATGGCTACC |
| | AAGGCGAAGAATCTTTATTTAACAAAGCATACTATGGCAAAAGCACAT |
| | CATTCTTCCGTCAAGAAAGTCAAAAACTTCTGCAAAGCGATAAAAAAC |
| | GCACGGCTGAGTTAGCAAACGGCGCTCTCGGTATGATTGAGCTAAACG |
| | ATGATTACACACTGAAAAAAGTGATGAAACCGCTGATTGCATCTAACA |
| | CAGTAACAGATGAAATTGAACGCGCGAACGTCTTTAAAATGAACGGCA |
| | AATGGTACCTGTTCACTGACTCCCGCGGATCAAAAATGACGATTGACG |
| | GCATTACGTCTAACGATATTTACATGCTTGGTTATGTTTCTAATTCTT |
| | TAACTGGCCCATACAAGCCGCTGAACAAAACTGGCCTTGTGTTAAAAA |
| | TGGATCTTGATCCTAACGATGTAACCTTTACTTACTCACACTTCGCTG |
| | TACCTCAAGCGAAAGGAAACAATGTCGTGATTACAAGCTATATGACAA |
| | ACAGAGGATTCTACGCAGACAAACAATCAACGTTTGCGCCGAGCTTCC |
| | TGCTGAACATCAAAGGCAAGAAAACATCTGTTGTCAAAGACAGCATCC |
| | TTGAACAAGGACAATTAACAGTTAACAAATAAAAACGCAAAAGAAAAT |
| | GCCGATGGGTACCGAGCGAAATGACCGACCAAGCGACGCCCAACCTGC |
| | CATCACGAGATTTCGATTCCACCGCCGCCTTCTATGAAAGGTTGGGCT |
| | TCGGAATCGTTTTCCGGGACGCCCTCGCGGACGTGCTCATAGTCCACG |
| | ACGCCCGTGATTTTGTAGCCCTGGCCGACGGCCAGCAGGTAGGCCGAC |
| | AGGCTCATGCCGGCCGCCGCCGCCTTTTCCTCAATCGCTCTTCGTTCG |
| | TCTGGAAGGCAGTACACCTTGATAGGTGGGCTGCCCTTCCTGGTTGGC |
| | TTGGTTTCATCAGCCATCCGCTTGCCCTCATCTGTTACGCCGGCGGTA |
| | GCCGGCCAGCCTCGCAGAGCAGGATTCCCGTTGAGCACCGCCAGGTGC |
| | GAATAAGGGACAGTGAAGAAGGAACACCCGCTCGCGGGTGGGCCTACT |
| | TCACCTATCCTGCCCGGCTGACGCCGTTGGATACACCAAGGAAAGTCT |
| | ACACGAACCCTTTGGCAAAATCCTGTATATCGTGCGAAAAAGGATGGA |
| | TATACCGAAAAAATCGCTATAATGACCCCGAAGCAGGGTTATGCAGCG |
| | GAAAAGCGCTGCTTCCCTGCTGTTTTGTGGAATATCTACCGACTGGAA |
| | ACAGGCAAATGCAGGAAATTACTGAACTGAGGGGACAGGCGAGAGACG |
| | ATGCCAAAGAGCTCCTGAAAATCTCGATAACTCAAAAAATACGCCCGG |
| | TAGTGATCTTATTTCATTATGGTGAAAGTTGGAACCTCTTACGTGCCG |
| | ATCAACGTCTCATTTTCGCCAAAAGTTGGCCCAGGGCTTCCCGGTATC |
| | AACAGGGACACCAGGATTTATTTATTCTGCGAAGTGATCTTCCGTCAC |
| | AGGTATTTATTCGGCGCAAAGTGCGTCGGGTGATGCTGCCAACTTACT |
| | GATTTAGTGTATGATGGTGTTTTTGAGGTGCTCCAGTGGCTTCTGTTT |
| | CTATCAGCTCCTGAAAATCTCGATAACTCAAAAAATACGCCCGGTAGT |
| | GATCTTATTTCATTATGGTGAAAGTTGGAACCTCTTACGTGCCGATCA |
| | ACGTCTCATTTTCGCCAAAAGTTGGCCCAGGGCTTCCCGGTATCAACA |
| | GGGACACCAGGATTTATTTATTCTGCGAAGTGATCTTCCGTCACAGGT |
| | ATTTATTCGGCGCAAAGTGCGTCGGGTGATGCTGCCAACTTACTGATT |
| | TAGTGTATGATGGTGTTTTTGAGGTGCTCCAGTGGCTTCTGTTTCTAT |
| | CAGGGCTGGATGATCCTCCAGCGCGGGGATCTCATGCTGGAGTTCTTC |
| | GCCCACCCCAAAAGGATCTAGGTGAAGATCCTTTTTGATAATCTCATG |
| | ACCAAAATCCCTTAACGTGAGTTTTCGTTCCACTGAGCGTCAGACCCC |
| | GTAGAAAAGATCAAAGGATCTTCTTGAGATCCTTTTTTTCTGCGCGTA |
| | ATCTGCTGCTTGCAAACAAAAAAACCACCGCTACCAGCGGTGGTTTGT |
| | TTGCCGGATCAAGAGCTACCAACTCTTTTTCCGAAGGTAACTGGCTTC |
| | AGCAGAGCGCAGATACCAAATACTGTTCTTCTAGTGTAGCCGTAGTTA |
| | GGCCACCACTTCAAGAACTCTGTAGCACCGCCTACATACCTCGCTCTG |
| | CTAATCCTGTTACCAGTGGCTGCTGCCAGTGGCGATAAGTCGTGTCTT |
| | ACCGGGTTGGACTCAAGACGATAGTTACCGGATAAGGCGCAGCGGTCG |
| | GGCTGAACGGGGGGTTCGTGCACACAGCCCAGCTTGGAGCGAACGACC |
| | TACACCGAACTGAGATACCTACAGCGTGAGCTATGAGAAAGCGCCACG |
| | CTTCCCGAAGGGAGAAAGGCGGACAGGTATCCGGTAAGCGGCAGGGTC |
| | GGAACAGGAGAGCGCACGAGGGAGCTTCCAGGGGGAAACGCCTGGTAT |
| | CTTTATAGTCCTGTCGGGTTTCGCCACCTCTGACTTGAGCGTCGATTT |
| | TTGTGATGCTCGTCAGGGGGGCGGAGCCTATGGAAAAACGCCAGCAAC |
| | GCGGCCTTTTTACGGTTCCTGGCCTTTTGCTGGCCTTTTGCTCACATG |
| | TTCTTTCCTGCGTTATCCCCTGATTCTGTGGATAACCGTATTACCGCC |
| | TTTGAGTGAGCTGATACCGCTCGCCGCAGCCGAACGACCGAGCGCAGC |
| | GAGTCAGTGAGCGAGGAAGCGGAAGAGCGCCCAATACGCAAACCGCCT |
| | CTCCCCGCGCGTTGGCCGATTCATTAATGCAGCTGGCACGACAGGTTT |
| | CCCGACTGGAAAGCGGGCAGTGAGCGCAACGCAATTAATGTGAGTTAG |
| | CTCACTCATTAGGCACCCCAGGCTTTACACTTTATGCTTCCGGCTCGT |
| | ATGTTGTGTGGAATTGTGAGCGGATAACAATTTCACACAGGAAACAGC |
| | TATGACATGATTACGAATTCGAGCTCGGTACCCGGGGATCCTCTAGAG |
| | TCGACCTGCAGGCATGCAAGCTTGGCACTGGCCGTCGTTTTACAACGT |
| | CGTGACTGGGAAAACCCTGGCGTTACCCAACTTAATCGCCTTGCAGCA |
| | CATCCCCCTTTCGCCAGCTGGCGTAATAGCGAAGAGGCCCGCACCGAT |
| | CGCCCTTCCCAACAGTTGCGCAGCCTGAATGGCGAATGGCGATAAGCT |
| | AGCTTCACGC-3′ |

To evaluate autotrophic growth, a single colony of the DhoxKGZ strain from a freshly streaked TSB plate was used to inoculate 3 mL TSB containing 50 mg/L chloramphenicol in a 14 mL Falcon round bottom polystyrene test tube with snap cap. Following overnight incubation at 30° C. and 200 rpm in a Thermo MAXQ shaker, 1 mL of culture was used to inoculate 100 mL LB in a 200 mL Schott bottle. Cells were grown at 30° C. and 200 rpm until an optical density of ˜0.3-0.4 was reached. 100 mL of this culture was used to inoculate a 1.4-L Infors HT Multifors 2 CSTR containing 600 mL of 2× startup media. The reactor was incubated at 30° C. and initiated with 250 rpm agitation and 150 nccm gas flow (3.14% 02, 41% H2, 3% CO2, 52.86% N2). Agitation and gas flow were ramped up to 1450 rpm and 750 nccm as the culture grew. When OD600 exceeded 0.5, the culture was turned continuous using 4× media with 7 μL/hr Pluronic 31R1 antifoam. The feed oxygen percentage was gradually increased to promote biomass production, with the balance taken off nitrogen percentage, subject to the constraint that the outlet oxygen percentage remain below 4.5% as a safety measure.

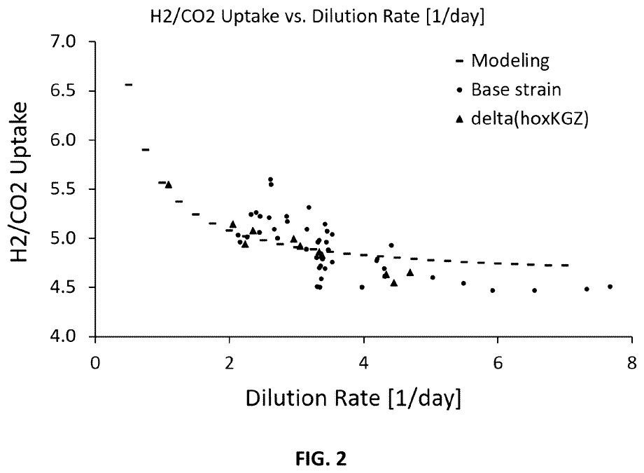

Once the culture was well-established, performance (H2/CO2 uptake) was evaluated at a varying dilution rates (FIG. 2). Gas samples from the reactor were plumbed via 305 stainless steel to a stream selection valve controlled by a microGC (manufacturer: Qmicro). Samples were then analyzed on a Rt-U BOND XP PLOT column under isothermal conditions (70° C.) via a thermal conductivity detector (TCD).

### Example 2: Improving Ethylene Production Through the Use of a DhoxKGZ Background to Increasing Intracellular Oxygen Availability

The gene coding for ethylene forming enzyme was codon-adapted and synthesized for expression in Cupriavidus necator. The adapted gene along with the native CBB-operon promoter were cloned into the broad host range expression vector pBBR1MCS2. The resulting products were used to transform E. coli and positive clones identified by PCR were confirmed by DNA sequencing. The sequence confirmed plasmid was then transformed into Cupriavidus necator DhoxKGZ via electroporation and selected on tryptic soy broth (TSB) agar plates containing 50 mg/L chloramphenicol. Transformants containing the pBBR1-Efe plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs

A single colony from a freshly streaked TSB plate was used to inoculate 3 mL TSB containing 50 mg/L chloramphenicol in a 14 mL Falcon round bottom polystyrene test tube with snap cap. Following overnight incubation at 30° C. and 200 rpm in a Thermo MAXQ shaker, 1 mL of culture was used to inoculate 100 mL LB in a 200 mL Schott bottle. Cells were grown at 30° C. and 200 rpm until an optical density of ˜0.3-0.4 was reached.

100 mL of the above culture was used to inoculate a 1.4-L Infors HT Multifors 2 CSTR containing 600 mL of 2× startup media. The reactor was incubated at 30° C. and initiated with 250 rpm agitation and 150 nccm gas flow (3.14% 02, 41% H₂, 3% CO₂, 52.86% N₂). Agitation and gas flow were ramped up to 1450 rpm and 750 nccm as the culture grew. When OD₆₀₀ exceeded 0.5, the culture was turned continuous using 4×_media with 7 μL/hr Pluronic 31R1 antifoam. The feed oxygen percentage was gradually increased to promote biomass production, with the balance taken off nitrogen percentage, subject to the constraint that the outlet oxygen percentage remain below 4.5% as a safety measure. Gas samples from the reactor were plumbed via 305 stainless steel to a stream selection valve controlled by a microGC (manufacturer: Qmicro). Samples were then analyzed on a Rt-U BOND XP PLOT column under isothermal conditions (70° C.) via a thermal conductivity detector (TCD).

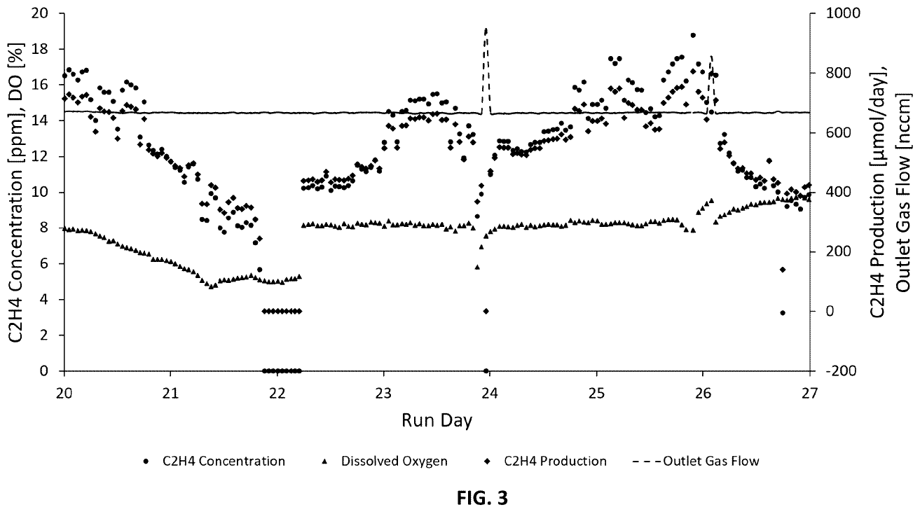

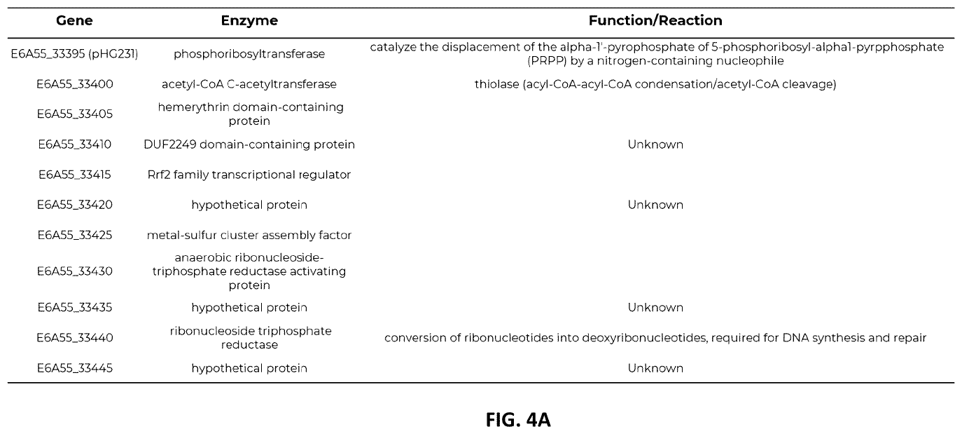

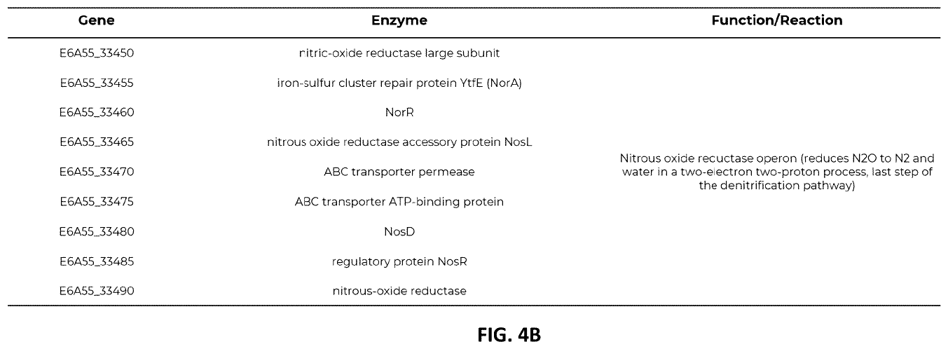

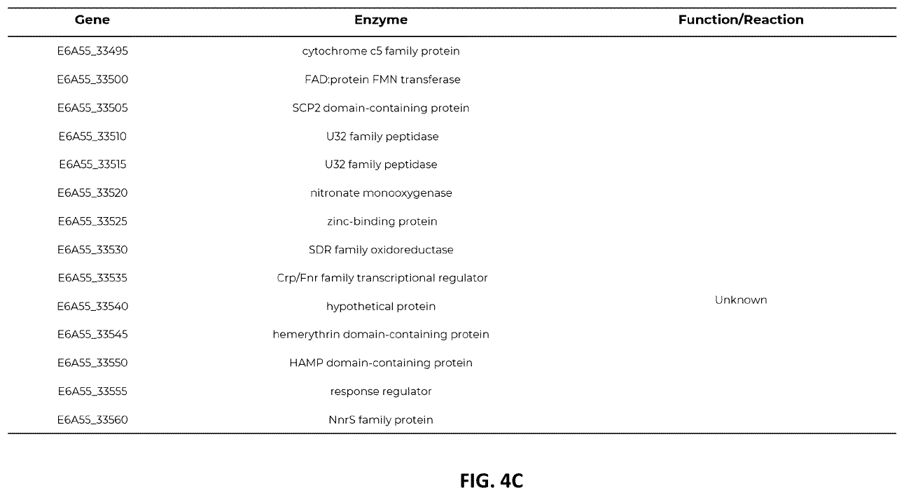

Once the culture was well-established, gas fractions were adjusted from O₂-limiting to H₂-limiting conditions such that a non-zero dissolved oxygen (DO) concentration was observed. Ethylene production varied as the system settled into steady-state and as gas fractions were adjusted, but production was maintained continuously (FIG. 3). In addition, the dissolved oxygen concentration remained stable ˜8%.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavour in any country.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (e.g., “or”) should be understood to mean either one, both, or any combination thereof of the alternatives. As used herein, the term “about” means+20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A recombinant C1-fixing microorganism comprising a disruptive mutation at a membrane bound hydrogenase gene locus.

2. The microorganism according to claim 1, wherein the disruptive mutation is a deletion.

3. The microorganism according to claim 1, wherein the membrane bound hydrogenase gene is hoxKGZ.

4. The microorganism according to claim 1, wherein the microorganism is selected from the group consisting of Cupriavidus necator and Ralstonia eutropha.

5. The strain according to claim 1, wherein the strain does not natively produce polyhydroxyalkanoates (PHAs).

6. The microorganism according to claim 4, wherein the Cupriavidus necator is selected from the group consisting of DSM 428, DSM 531, DSM 541, and DSM 34774.

7. The microorganism according to claim 6, wherein the microorganism is Cupriavidus necator DSM 34774 or a derivative thereof.

8. The microorganism according to claim 1, wherein the microorganism has at least 95% sequence identity to SEQ ID NOs: 1-3.

9. A method for the continuous production of a product, the method comprising culturing the microorganism of claim 1 in the presence of a gaseous substrate.

10. The method according to claim 9, wherein the gaseous substrate comprises an industrial waste product or off-gas.

11. The method according to claim 10, further comprising an energy source.

12. The method according to claim 11, wherein the energy source is provided intermittently.

13. The method according to claim 9, wherein the gaseous substrate comprises CO₂ and an energy source.

14. The method according to claim 13, wherein the energy source is H₂.

15. The method according to claim 13, wherein the gaseous substrate further comprises H₂, O₂, or both.

16. The method according to claim 9, wherein the microorganism comprises a reduced H₂:CO₂ uptake ratio by minimizing H₂ oxidation to water.

17. The method according to claim 9, wherein the microorganism comprises an increase in the intracellular oxygen availability.

18. A method for obtaining a product from a circular process, wherein the method comprises:
a) generating electric energy using a renewable and/or non-renewable source,
b) electrolyzing water or CO₂ to produce at least H₂, O₂ or CO;
c) passing at least one of the H₂, O₂ or CO to a bioreactor containing a culture comprising a liquid nutrient medium and a recombinant C1-fixing microorganism according to claim 1; and
d) fermenting the culture to produce at least the product.

19. The method according to claim 18, wherein disruptive mutation is a deletion and the membrane bound hydrogenase is hoxKGZ.

20. The method according to claim 18, wherein the C1-fixing microorganism is selected from the group consisting of DSM 428, DSM 531, DSM 541, and DSM 34774.
